(19) United States
(12) Patent Application Publication
(10) Pub. No.: US 2025/0241952 A1
(43) Pub. Date: Jul. 31, 2025

(54) CD83 DUAL CAR T CELLS

(71) Applicants: H. LEE MOFFITT CANCER CENTER AND RESEARCH INSTITUTE INC., Tampa, FL (US); REGENTS OF THE UNIVERSITY OF MINNESOTA, Minneapolis, MN (US)

(72) Inventors: Marco Davila, Tampa (FL); Brian Betts, Blaine, MN (US)

(21) Appl. No.: 18/856,207

(22) PCT Filed: Mar. 7, 2023
(86) PCT No.: PCT/US2023/063832

§ 371 Date: Oct. 11, 2024

Related U.S. Application Data
(60) Provisional application No. 63/330,417, filed on Apr. 13, 2022.

(51) Int. Cl. A61K 35/17; A61K 40/11; A61K 40/15; A61K 40/31; A61K 40/42; A61P 35/00; C07K 16/28

(52) U.S. Cl. CPC A61K 35/17; A61K 40/11; A61K 40/15; A61K 40/31; A61K 40/4203; A61K 40/421; A61K 40/4217; A61P 35/00; C07K 16/2803; C07K 16/2818; C07K 16/2827; C07K 16/2851; C07K 16/2863; C07K 16/2866; A61K 2239/13; A61K 2239/17; A61K 2239/21; A61K 2239/29; A61K 2239/48; C07K 2317/565; C07K 2317/622

(57) ABSTRACT

Dual-chimeric antigen receptor (CAR) cell systems are disclosed that can be used with adoptive cell transfer to target and kill cancers expressing tumor antigens (“TA”) that are also expressed on healthy hematopoietic cells. In some embodiments, the dual CAR cell expresses a first CAR polypeptide that contains in an ectodomain a binding agent that can selectively bind CD83 on CD83-expressing cancer cells (“anti-CD83 binding agent”), and a second CAR polypeptide that contains in an ectodomain an antigen-binding agent that can bind a second tumor antigen that is expressed on both the cancer and healthy hematopoietic cells (“anti-TA binding agent”), such as CD33, CLEC12A, CD123, or FLT3.

### CROSS-REFERENCE TO RELATED APPLICATIONS

This application claims benefit of U.S. Provisional Application No. 63/330,417, filed Apr. 13, 2022, which is hereby incorporated herein by reference in its entirety.

### SEQUENCE LISTING

This application contains a sequence listing filed in ST.26 format entitled “320805_2020_Sequence_Listing” created on Jan. 20, 2023. The content of the sequence listing is incorporated herein in its entirety.

### BACKGROUND OF THE INVENTION

CD33 is a widely studied acute myeloid leukemia (AML) CAR-T target, currently in several clinical trials. However, since CD33 is also expressed on hematopoietic cells, alloHCT is required within 6 weeks of infusion with CD33 CAR-T cells. This is therefore not an option for transplant-ineligible patients or patients without a donor. Thus, there is a critical unmet need in access to care for AML patients.

### SUMMARY OF THE INVENTION

Dual-chimeric antigen receptor (CAR) cell systems are disclosed that can be used with adoptive cell transfer to target and kill cancers expressing tumor antigens (“TA”) that are also expressed on healthy hematopoietic cells. In some embodiments, the dual CAR cell expresses a first CAR polypeptide that contains in an ectodomain a binding agent that can selectively bind CD83 on CD83-expressing cancer cells (“anti-CD83 binding agent”), and a second CAR polypeptide that contains in an ectodomain an antigen-binding agent that can bind a second tumor antigen that is expressed on both the cancer and healthy hematopoietic cells (“anti-TA binding agent”), such as CD33, CLEC12A, CD123, CD38, or FLT3.

Also disclosed is an immune effector cell genetically modified to express the disclosed CAR polypeptide(s). In some embodiments, the immune effector cell can be selected from the group consisting of alpha-beta T cells, gamma-delta T cells, Natural Killer (NK) cells, Natural Killer T (NKT) cells, B cells, innate lymphoid cells (ILCs), cytokine induced killer (CIK) cells, cytotoxic T lymphocytes (CTLs), lymphokine activated killer (LAK) cells, and regulatory T cells (Tregs).

The anti-CD83 binding agent and/or anti-TA binding agent is in some embodiments an antibody fragment that specifically binds CD83 or the TA, respectively. For example, the antigen binding domain can be a Fab or a single-chain variable fragment (scFv) of an antibody that specifically binds CD83 or the TA. The binding agent is in some embodiments an aptamer that specifically binds CD83 or the TA. For example, the binding agent can be a peptide aptamer selected from a random sequence pool based on its ability to bind CD83 or the TA. The binding agent can also be a natural ligand of CD83 or the TA, or a variant and/or fragment thereof capable of binding CD83 or the TA.

In some embodiments, the first and second CAR polypeptides have incomplete endodomains and only function when both CARs bind their target. For example, the CAR polypeptide can contain only an intracellular signaling domain or a co-stimulatory domain, but not both. In some embodiments, one CAR can include only the CD3ζ domain, and the other CAR can include only the co-stimulatory domain(s). In these embodiments, dual CAR T cell activation requires co-expression and activation by binding to both targets. In these embodiments, the immune effector cell is not activated unless it and a second CAR polypeptide (or endogenous T-cell receptor) that contains the missing domain both bind their respective antigens. Therefore, in some embodiments, the first CAR polypeptide contains a CD3 zeta (CD3ζ) signaling domain but does not contain a costimulatory signaling region (CSR). In other embodiments, the first CAR polypeptide contains the cytoplasmic domain of CD28, 4-1BB, or a combination thereof, but does not contain a CD3 zeta (CD3ζ) signaling domain (SD).

Also disclosed are isolated nucleic acid sequences encoding the disclosed CAR polypeptides, vectors comprising these isolated nucleic acids, and immune effector cells containing these vectors.

Also disclosed is a method of providing an anti-tumor immunity in a subject with a cancer that expresses CD83 and the TA that involves administering to the subject an effective amount of an immune effector cell genetically modified with a disclosed first and second CAR polypeptides. In some cases, the cancer can be any CD83/TA co-expressing malignancy. AML is known to express CD33, CD123, FLT3, CD38, and/or CLEC12A. Unlike CD83-CD33, CD123, FLT3, CD38, and/or CLEC12A are often expressed on AML blasts as well as healthy stem cells or circulating myeloid cells. In many cases the use of CD33 CAR T requires a stem cell transplant within 6 weeks of cell infusion. This is not always possible for medically feasible for patients. CD83 is unique in that it is expressed on the majority of AML blasts, but restricted from hematopoietic stem cells, myeloid precursors in the marrow, and peripheral neutrophils. Therefore, an ‘AND’ gated bicistronic design that codes for two separate CAR proteins (e.g., CD83 and CD33) could overcome the risk for myeloid aplasia. In one embodiment, the CD83 CAR T would hold CD3 zeta required for T cell activation and the CD33 (or CD123, FLT3, CD38, or CLEC12A) CAR would hold co-stimulation. Only when such a dual ‘AND’ gated CAR T engages a target cell with both antigens present, will the CAR T kill.

The details of one or more embodiments of the invention are set forth in the accompanying drawings and the description below. Other features, objects, and advantages of the invention will be apparent from the description and drawings, and from the claims.

### BRIEF DESCRIPTION OF FIGURES

FIGS. 1A to 1D show CD83 is expressed on B cell malignancies and readily killed by CD83 CAR T. CD19, CD83, CD20, and CD22 expression is shown among healthy peripheral B cells versus B cell malignancies (FIG. 1A) and Nalm6, Daudi, and Raji cell lines (FIG. 1B). FIGS. 1C and 1D show xCELLigence cytotoxicity assays show CD83 CAR T significantly kill Nalm6 and Raji target cells. *P<0.05, **P=0.001-0.01, ***P=0.0001-0.001, and ****P<0.0001. NS=not significant.

FIGS. 2A to 2C show human anti-CD19/CD83 CAR T design. FIG. 2A shows loop and tandem ‘OR’ logic gated CD19/CD83 CAR designs. FIG. 2B contains contour plots show transduction efficiency of CD19, CD83, and ‘OR’ gated CD19/CD83 CAR T. FIG. 2C shows CD19 versus CD83 expression on target Raji cells after overnight co-culture with CAR T cells (1:1). CD19 CAR T leads to decreased CD19 expression on the target cell.

FIGS. 3A and 3B show CD19/CD83 CAR T provide enhanced anti-leukemia potency and overcome CD19 antigen escape. xCELLigence assays show CD19/CD83 CAR T cytolytic potency against CD19 high (FIG. 3A) and CD19 deficient (FIG. 3B) Raji cell lines, compared to untransduced (UT), CD19, or CD83 mono-gated CAR T (Effector to target ratio 3:1). ****P<0.0001.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

The term “amino acid sequence” refers to a list of abbreviations, letters, characters or words representing amino acid residues. The amino acid abbreviations used herein are conventional one letter codes for the amino acids and are expressed as follows: A, alanine; B, asparagine or aspartic acid; C, cysteine; D aspartic acid; E, glutamate, glutamic acid; F, phenylalanine; G, glycine; H histidine; I isoleucine; K, lysine; L, leucine; M, methionine; N, asparagine; P, proline; Q, glutamine; R, arginine; S, serine; T, threonine; V, valine; W, tryptophan; Y, tyrosine; Z, glutamine or glutamic acid.

The term “antibody” refers to an immunoglobulin, derivatives thereof which maintain specific binding ability, and proteins having a binding domain which is homologous or largely homologous to an immunoglobulin binding domain. These proteins may be derived from natural sources, or partly or wholly synthetically produced. An antibody may be monoclonal or polyclonal. The antibody may be a member of any immunoglobulin class from any species, including any of the human classes: IgG, IgM, IgA, IgD, and IgE. In exemplary embodiments, antibodies used with the methods and compositions described herein are derivatives of the IgG class. In addition to intact immunoglobulin molecules, also included in the term “antibodies” are fragments or polymers of those immunoglobulin molecules, and human or humanized versions of immunoglobulin molecules that selectively bind the target antigen.

The term “antibody fragment” refers to any derivative of an antibody which is less than full-length. In exemplary embodiments, the antibody fragment retains at least a significant portion of the full-length antibody's specific binding ability. Examples of antibody fragments include, but are not limited to, Fab, Fab′, F(ab′)2, scFv, Fv, dsFv diabody, Fc, and Fd fragments. The antibody fragment may be produced by any means. For instance, the antibody fragment may be enzymatically or chemically produced by fragmentation of an intact antibody, it may be recombinantly produced from a gene encoding the partial antibody sequence, or it may be wholly or partially synthetically produced. The antibody fragment may optionally be a single chain antibody fragment. Alternatively, the fragment may comprise multiple chains which are linked together, for instance, by disulfide linkages. The fragment may also optionally be a multimolecular complex. A functional antibody fragment will typically comprise at least about 50 amino acids and more typically will comprise at least about 200 amino acids.

The term “antigen binding site” refers to a region of an antibody that specifically binds an epitope on an antigen.

The term “aptamer” refers to oligonucleic acid or peptide molecules that bind to a specific target molecule. These molecules are generally selected from a random sequence pool. The selected aptamers are capable of adapting unique tertiary structures and recognizing target molecules with high affinity and specificity. A “nucleic acid aptamer” is a DNA or RNA oligonucleic acid that binds to a target molecule via its conformation, and thereby inhibits or suppresses functions of such molecule. A nucleic acid aptamer may be constituted by DNA, RNA, or a combination thereof. A “peptide aptamer” is a combinatorial protein molecule with a variable peptide sequence inserted within a constant scaffold protein. Identification of peptide aptamers is typically performed under stringent yeast dihybrid conditions, which enhances the probability for the selected peptide aptamers to be stably expressed and correctly folded in an intracellular context.

The term “carrier” means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject.

The term “chimeric molecule” refers to a single molecule created by joining two or more molecules that exist separately in their native state. The single, chimeric molecule has the desired functionality of all of its constituent molecules. One type of chimeric molecules is a fusion protein.

The term “engineered antibody” refers to a recombinant molecule that comprises at least an antibody fragment comprising an antigen binding site derived from the variable domain of the heavy chain and/or light chain of an antibody and may optionally comprise the entire or part of the variable and/or constant domains of an antibody from any of the Ig classes (for example IgA, IgD, IgE, IgG, IgM and IgY).

The term “epitope” refers to the region of an antigen to which an antibody binds preferentially and specifically. A monoclonal antibody binds preferentially to a single specific epitope of a molecule that can be molecularly defined. In the present invention, multiple epitopes can be recognized by a multispecific antibody.

The term “fusion protein” refers to a polypeptide formed by the joining of two or more polypeptides through a peptide bond formed between the amino terminus of one polypeptide and the carboxyl terminus of another polypeptide. The fusion protein can be formed by the chemical coupling of the constituent polypeptides or it can be expressed as a single polypeptide from nucleic acid sequence encoding the single contiguous fusion protein. A single chain fusion protein is a fusion protein having a single contiguous polypeptide backbone. Fusion proteins can be prepared using conventional techniques in molecular biology to join the two genes in frame into a single nucleic acid, and then expressing the nucleic acid in an appropriate host cell under conditions in which the fusion protein is produced.

The term “Fab fragment” refers to a fragment of an antibody comprising an antigen-binding site generated by cleavage of the antibody with the enzyme papain, which cuts at the hinge region N-terminally to the inter-H-chain disulfide bond and generates two Fab fragments from one antibody molecule.

The term “F(ab′)2 fragment” refers to a fragment of an antibody containing two antigen-binding sites, generated by cleavage of the antibody molecule with the enzyme pepsin which cuts at the hinge region C-terminally to the inter-H-chain disulfide bond.

The term “Fc fragment” refers to the fragment of an antibody comprising the constant domain of its heavy chain.

The term “Fv fragment” refers to the fragment of an antibody comprising the variable domains of its heavy chain and light chain.

“Gene construct” refers to a nucleic acid, such as a vector, plasmid, viral genome or the like which includes a “coding sequence” for a polypeptide or which is otherwise transcribable to a biologically active RNA (e.g., antisense, decoy, ribozyme, etc), may be transfected into cells, e.g. in certain embodiments mammalian cells, and may cause expression of the coding sequence in cells transfected with the construct. The gene construct may include one or more regulatory elements operably linked to the coding sequence, as well as intronic sequences, polyadenylation sites, origins of replication, marker genes, etc.

The term “identity” refers to sequence identity between two nucleic acid molecules or polypeptides. Identity can be determined by comparing a position in each sequence which may be aligned for purposes of comparison. When a position in the compared sequence is occupied by the same base, then the molecules are identical at that position. A degree of similarity or identity between nucleic acid or amino acid sequences is a function of the number of identical or matching nucleotides at positions shared by the nucleic acid sequences. Various alignment algorithms and/or programs may be used to calculate the identity between two sequences, including FASTA, or BLAST which are available as a part of the GCG sequence analysis package (University of Wisconsin, Madison, Wis.), and can be used with, e.g., default setting. For example, polypeptides having at least 70%, 85%, 90%, 95%, 98% or 99% identity to specific polypeptides described herein and preferably exhibiting substantially the same functions, as well as polynucleotide encoding such polypeptides, are contemplated. Unless otherwise indicated a similarity score will be based on use of BLOSUM62. When BLASTP is used, the percent similarity is based on the BLASTP positives score and the percent sequence identity is based on the BLASTP identities score. BLASTP “Identities” shows the number and fraction of total residues in the high scoring sequence pairs which are identical; and BLASTP “Positives” shows the number and fraction of residues for which the alignment scores have positive values and which are similar to each other. Amino acid sequences having these degrees of identity or similarity or any intermediate degree of identity of similarity to the amino acid sequences disclosed herein are contemplated and encompassed by this disclosure. The polynucleotide sequences of similar polypeptides are deduced using the genetic code and may be obtained by conventional means, in particular by reverse translating its amino acid sequence using the genetic code.

The term “linker” is art-recognized and refers to a molecule or group of molecules connecting two compounds, such as two polypeptides. The linker may be comprised of a single linking molecule or may comprise a linking molecule and a spacer molecule, intended to separate the linking molecule and a compound by a specific distance.

The term “multivalent antibody” refers to an antibody or engineered antibody comprising more than one antigen recognition site. For example, a “bivalent” antibody has two antigen recognition sites, whereas a “tetravalent” antibody has four antigen recognition sites. The terms “monospecific”, “bispecific”, “trispecific”, “tetraspecific”, etc. refer to the number of different antigen recognition site specificities (as opposed to the number of antigen recognition sites) present in a multivalent antibody. For example, a “monospecific” antibody's antigen recognition sites all bind the same epitope. A “bispecific” antibody has at least one antigen recognition site that binds a first epitope and at least one antigen recognition site that binds a second epitope that is different from the first epitope. A “multivalent monospecific” antibody has multiple antigen recognition sites that all bind the same epitope. A “multivalent bispecific” antibody has multiple antigen recognition sites, some number of which bind a first epitope and some number of which bind a second epitope that is different from the first epitope.

The term “nucleic acid” refers to a natural or synthetic molecule comprising a single nucleotide or two or more nucleotides linked by a phosphate group at the 3′ position of one nucleotide to the 5′ end of another nucleotide. The nucleic acid is not limited by length, and thus the nucleic acid can include deoxyribonucleic acid (DNA) or ribonucleic acid (RNA).

The term “operably linked to” refers to the functional relationship of a nucleic acid with another nucleic acid sequence. Promoters, enhancers, transcriptional and translational stop sites, and other signal sequences are examples of nucleic acid sequences operably linked to other sequences. For example, operable linkage of DNA to a transcriptional control element refers to the physical and functional relationship between the DNA and promoter such that the transcription of such DNA is initiated from the promoter by an RNA polymerase that specifically recognizes, binds to and transcribes the DNA.

The terms “peptide,” “protein,” and “polypeptide” are used interchangeably to refer to a natural or synthetic molecule comprising two or more amino acids linked by the carboxyl group of one amino acid to the alpha amino group of another.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

The terms “polypeptide fragment” or “fragment”, when used in reference to a particular polypeptide, refers to a polypeptide in which amino acid residues are deleted as compared to the reference polypeptide itself, but where the remaining amino acid sequence is usually identical to that of the reference polypeptide. Such deletions may occur at the amino-terminus or carboxy-terminus of the reference polypeptide, or alternatively both. Fragments typically are at least about 5, 6, 8 or 10 amino acids long, at least about 14 amino acids long, at least about 20, 30, 40 or 50 amino acids long, at least about 75 amino acids long, or at least about 100, 150, 200, 300, 500 or more amino acids long. A fragment can retain one or more of the biological activities of the reference polypeptide. In various embodiments, a fragment may comprise an enzymatic activity and/or an interaction site of the reference polypeptide. In another embodiment, a fragment may have immunogenic properties.

The term “protein domain” refers to a portion of a protein, portions of a protein, or an entire protein showing structural integrity; this determination may be based on amino acid composition of a portion of a protein, portions of a protein, or the entire protein.

The term “single chain variable fragment or scFv” refers to an Fv fragment in which the heavy chain domain and the light chain domain are linked. One or more scFv fragments may be linked to other antibody fragments (such as the constant domain of a heavy chain or a light chain) to form antibody constructs having one or more antigen recognition sites.

A “spacer” as used herein refers to a peptide that joins the proteins comprising a fusion protein. Generally a spacer has no specific biological activity other than to join the proteins or to preserve some minimum distance or other spatial relationship between them. However, the constituent amino acids of a spacer may be selected to influence some property of the molecule such as the folding, net charge, or hydrophobicity of the molecule.

The term “specifically binds”, as used herein, when referring to a polypeptide (including antibodies) or receptor, refers to a binding reaction which is determinative of the presence of the protein or polypeptide or receptor in a heterogeneous population of proteins and other biologics. Thus, under designated conditions (e.g. immunoassay conditions in the case of an antibody), a specified ligand or antibody “specifically binds” to its particular “target” (e.g. an antibody specifically binds to an endothelial antigen) when it does not bind in a significant amount to other proteins present in the sample or to other proteins to which the ligand or antibody may come in contact in an organism. Generally, a first molecule that “specifically binds” a second molecule has an affinity constant (Ka) greater than about 10⁵ M⁻¹ (e.g., 10⁶ M⁻¹, 10⁷ M⁻¹, 10⁸ M⁻¹, 10⁹ M⁻¹, 10¹⁰ M⁻¹, 10¹¹ M⁻¹, and 10¹² M⁻¹ or more) with that second molecule.

The term “specifically deliver” as used herein refers to the preferential association of a molecule with a cell or tissue bearing a particular target molecule or marker and not to cells or tissues lacking that target molecule. It is, of course, recognized that a certain degree of non-specific interaction may occur between a molecule and a non-target cell or tissue. Nevertheless, specific delivery, may be distinguished as mediated through specific recognition of the target molecule. Typically specific delivery results in a much stronger association between the delivered molecule and cells bearing the target molecule than between the delivered molecule and cells lacking the target molecule.

The term “subject” refers to any individual who is the target of administration or treatment. The subject can be a vertebrate, for example, a mammal. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The terms “transformation” and “transfection” mean the introduction of a nucleic acid, e.g., an expression vector, into a recipient cell including introduction of a nucleic acid to the chromosomal DNA of said cell.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “variant” refers to an amino acid or peptide sequence having conservative amino acid substitutions, non-conservative amino acid substitutions (i.e. a degenerate variant), substitutions within the wobble position of each codon (i.e. DNA and RNA) encoding an amino acid, amino acids added to the C-terminus of a peptide, or a peptide having 60%, 70%, 80%, 90%, 95%, 96%, 97%, 98%, 99% sequence identity to a reference sequence.

The term “vector” refers to a nucleic acid sequence capable of transporting into a cell another nucleic acid to which the vector sequence has been linked. The term “expression vector” includes any vector, (e.g., a plasmid, cosmid or phage chromosome) containing a gene construct in a form suitable for expression by a cell (e.g., linked to a transcriptional control element).

### Chimeric Antigen Receptors (CARs)

CARs generally incorporate an antigen recognition domain from the single-chain variable fragments (scFv) of a monoclonal antibody (mAb) with transmembrane signaling motifs involved in lymphocyte activation (Sadelain M, et al. Nat Rev Cancer 2003 3:35-45). Disclosed herein is a CD83-specific chimeric antigen receptor (CAR) that can be that can be expressed in immune effector cells to suppress alloreactive donor cells.

The disclosed CAR is generally made up of three domains: an ectodomain, a transmembrane domain, and an endodomain. The ectodomain comprises the CD83-binding region and is responsible for antigen recognition. It also optionally contains a signal peptide (SP) so that the CAR can be glycosylated and anchored in the cell membrane of the immune effector cell. The transmembrane domain (TD), is as its name suggests, connects the ectodomain to the endodomain and resides within the cell membrane when expressed by a cell. The endodomain is the business end of the CAR that transmits an activation signal to the immune effector cell after antigen recognition. For example, the endodomain can contain an intracellular signaling domain (ISD) and optionally a co-stimulatory signaling region (CSR).

A “signaling domain (SD)” generally contains immunoreceptor tyrosine-based activation motifs (ITAMs) that activate a signaling cascade when the ITAM is phosphorylated. The term “co-stimulatory signaling region (CSR)” refers to intracellular signaling domains from costimulatory protein receptors, such as CD28, 41BB, and ICOS, that are able to enhance T-cell activation by T-cell receptors.

In some embodiments, a dual CAR T cell disclosed herein can have incomplete endodomains and only function when both CARs bind their target. Therefore, in some embodiments, the endodomain contains an SD or a CSR, but not both. In these embodiments, an immune effector cell containing the disclosed CAR is only activated if another CAR (or a T-cell receptor) containing the missing domain also binds its respective antigen. For example, one CAR can include only the CD3ζ domain, and the other CAR can include only the co-stimulatory domain(s). In these embodiments, dual CAR T cell activation requires co-expression and activation by binding to both targets. This is referred to herein as “AND-gating.”

In some embodiments, the disclosed first and second CARs are defined by the formula:

SP-CD83-HG-TM-CSR, and

SP-TA-HG-TM-SD; or

SP-CD83-HG-TM-SD, and

SP-TA-HG-TM-CSR;
wherein “SP” represents an optional signal peptide,
wherein “CD83” represents a CD83-binding region,
wherein “TA” represents a TA-binding region (e.g. anti-CD33, anti-CLEC12A, anti-CD123, anti-CD38, or anti-FLT3,
wherein “HG” represents an optional hinge domain,
wherein “TM” represents a transmembrane domain,
wherein “CSR” represents one or more co-stimulatory signaling regions,
wherein “SD” represents a signaling domain, and
wherein “-” represents a peptide bond or linker.

As disclosed herein, first CAR polypeptide contains in an ectodomain a binding agent that can selectively bind CD83 on CD83-expressing cancer cells (“anti-CD83 binding agent”), and the second CAR polypeptide contains in an ectodomain an antigen-binding agent that can bind a second tumor antigen that is expressed on both the cancer and healthy hematopoietic cells (“anti-TA binding agent”), such as CD33, CLEC12A, CD123, CD38, or FLT3.

### Anti-CD83

The anti-CD83 binding agent is in some embodiments an antibody fragment that specifically binds CD83. For example, the antigen binding domain can be a Fab or a single-chain variable fragment (scFv) of an antibody that specifically binds CD83. The anti-CD83 binding agent is in some embodiments an aptamer that specifically binds CD83. For example, the anti-CD83 binding agent can be a peptide aptamer selected from a random sequence pool based on its ability to bind CD83. The anti-CD83 binding agent can also be a natural ligand of CD83, or a variant and/or fragment thereof capable of binding CD83.

In some embodiments, the anti-CD83 scFv can comprise a variable heavy (V_{H}) domain having CDR1, CDR2 and CDR3 sequences and a variable light (V_{L}) domain having CDR1, CDR2 and CDR3 sequences.

For example, in some embodiments, the CDR1 sequence of the V_{H} domain comprises the amino acid sequence GFSITTGGYWWT (SEQ ID NO:1), SDGIS (SEQ ID NO:7), or SNAMI (SEQ ID NO:13); CDR2 sequence of the V_{H} domain comprises the amino acid sequence GYIFSSGNTNYNPSIKS (SEQ ID NO:2), IISSGGNTYYASWAKG (SEQ ID NO:8), or AMDSNSRTYYATWAKG (SEQ ID NO:14); CDR3 sequence of the V_{H} domain comprises the amino acid sequence CARAYGKLGFDY (SEQ ID NO:3), VVGGTYSI (SEQ ID NO:9), or GDGGSSDYTEM (SEQ ID NO:15); CDR1 sequence of the V_{L} comprises the amino acid sequence TLSSQHSTYTIG (SEQ ID NO:4), QSSQSVYNNDFLS (SEQ ID NO:10), or QSSQSVYGNNELS (SEQ ID NO:16); CDR2 sequence of the V_{L} domain comprises the amino acid sequence VNSDGSHSKGD (SEQ ID NO:5), YASTLAS (SEQ ID NO: 11), or QASSLAS (SEQ ID NO:17); and CDR3 sequence of the V_{L} domain comprises the amino acid sequence GSSDSSGYV (SEQ ID NO:6), TGTYGNSAWYEDA (SEQ ID NO:12), or LGEYSISADNH (SEQ ID NO:18).

For example, in some embodiments, the CDR1 sequence of the V_{H} domain comprises the amino acid sequence GFSITTGGYWWT (SEQ ID NO:1), CDR2 sequence of the V_{H} domain comprises the amino acid sequence GYIFSSGNTNYNPSIKS (SEQ ID NO:2), CDR3 sequence of the V_{H} domain comprises the amino acid sequence CARAYGKLGFDY (SEQ ID NO:3), CDR1 sequence of the V_{L} comprises the amino acid sequence TLSSQHSTYTIG (SEQ ID NO:4), CDR2 sequence of the V_{L} domain comprises the amino acid sequence VNSDGSHSKGD (SEQ ID NO:5), and CDR3 sequence of the V_{L} domain comprises the amino acid sequence GSSDSSGYV (SEQ ID NO:6).

For example, in some embodiments, the CDR1 sequence of the V_{H} domain comprises the amino acid sequence SDGIS (SEQ ID NO:7), CDR2 sequence of the V_{H} domain comprises the amino acid sequence IISSGGNTYYASWAKG (SEQ ID NO:8), CDR3 sequence of the V_{H} domain comprises the amino acid sequence VVGGTYSI (SEQ ID NO:9), CDR1 sequence of the V_{L} comprises the amino acid sequence QSSQSVYNNDFLS (SEQ ID NO:10), CDR2 sequence of the V_{L} domain comprises the amino acid sequence YASTLAS (SEQ ID NO:11), and CDR3 sequence of the V_{L} domain comprises the amino acid sequence TGTYGNSAWYEDA (SEQ ID NO:12).

For example, in some embodiments, the CDR1 sequence of the V_{H} domain comprises the amino acid sequence SNAMI (SEQ ID NO:13), CDR2 sequence of the V_{H} domain comprises the amino acid sequence AMDSNSRTYYATWAKG (SEQ ID NO:14), CDR3 sequence of the V_{H} domain comprises the amino acid sequence GDGGSSDYTEM (SEQ ID NO:15), CDR1 sequence of the V_{L} comprises the amino acid sequence QSSQSVYGNNELS (SEQ ID NO:16), CDR2 sequence of the V_{L} domain comprises the amino acid sequence QASSLAS (SEQ ID NO:17), and CDR3 sequence of the V_{L} domain comprises the amino acid sequence LGEYSISADNH (SEQ ID NO:18).

In some embodiments, the anti-CD83 scFv V_{H} domain comprises the amino acid sequence:

(SEQ ID NO: 19, VH-GBM00)
QVQLKESGPGLVKPSQSLSLTCSVTGFSITTGGYWWTWIRQFPGQKLEW
MGYIFSSGNTNYNPSIKSRISITRDTSKNQFFLQLNSVTTEGDTARYYC
ARAYGKLGFDYWGQGTLVTVSS,

and the anti-CD83 scFv V_{L} domain comprises the amino acid sequence:

(SEQ ID NO: 20, VL-GBM00)
QPVLTQSPSASASLGNSVKITCTLSSQHSTYTIGWYQQHPDKAPKYVMY
VNSDGSHSKGDGIPDRFSGSSSGAHRYLSISNIQPEDEADYFCGSSDSS
GYVFGSGTQLTVL.

In some embodiments, the anti-CD83 scFv V_{H} domain comprises the amino acid sequence:

(SEQ ID NO: 21, 20D04)
METGLRWLLLVAVLKGVQCQSVEESGGRLVTPGTPLTLTCTVSGFSLSN
NAINWVRQAPGKGLEWIGYIWSGGLTYYANWAEGRFTISKTSTTVDLKM
TSPTIEDTATYFCARGINNSALWGPGTLVTVSSGQPKAPSVFPLAPCCG
DTPSSTVTLGCLVKGYLPEPVTVTWNSGTLTNGVRTFPSVRQSSGLYSL
SSVVSVTSSSQPVTCNVAHPATNTKVDKTVAPSTCSKPTCPPPELLGGP
SVFIFPPKPKDTLMISRTPEVTCVVVDVSQDDPEVQFTWYINNEQVRTA
RPPLREQQFNSTIRVVSTLPIAHQDWLRGKEFKCKVHNKALPAPIEKTI
SKARGQPLEPKVYTMGPPREELSSRSVSLTCMINGFYPSDISVEWEKNG
KAEDNYKTTPAVLDSDGSYFLYNKLSVPTSEWQRGDVFTCSVMHEALHN
HYTQKSISRSPGK,

and the anti-CD83 scFv V_{L} domain comprises the amino acid sequence:

(SEQ ID NO: 22, 20D04)
MDMRAPTQLLGLLLLWLPGARCADVVMTQTPASVSAAVGGTVTINCQAS
ESISNYLSWYQQKPGQPPKLLIYRTSTLASGVSSRFKGSGSGTEYTLTI
SGVQCDDVATYYCQCTSGGKFISDGAAFGGGTEVWVKGDPVAPTVLLFP
PSSDEVATGTVTIVCVANKYFPDVTVTWEVDGTTQTTGIENSKTPQNSA
DCTYNLSSTLTLTSTQYNSHKEYTCKVTQGTTSVVQSFSRKNC.

In some embodiments, the anti-CD83 scFv V_{H} domain comprises the amino acid sequence:

(SEQ ID NO: 23, 11G05)
METGLRWLLLVAVLKGVQCQSVEESGGRLVTPGTPLTLTCTVSGFTISD
YDLSWVRQAPGEGLKYIGFIAIDGNPYYATWAKGRFTISKTSTTVDLKI
TAPTTEDTATYFCARGAGDLWGPGTLVTVSSGQPKAPSVFPLAPCCGDT
PSSTVTLGCLVKGYLPEPVTVTWNSGTLTNGVRTFPSVRQSSGLYSLSS
VVSVTSSSQPVTCNVAHPATNTKVDKTVAPSTCSKPTCPPPELLGGPSV
FIFPPKPKDTLMISRTPEVTCVVVDVSQDDPEVQFTWYINNEQVRTARP
PLREQQFNSTIRVVSTLPIAHQDWLRGKEFKCKVHNKALPAPIEKTISK
ARGQPLEPKVYTMGPPREELSSRSVSLTCMINGFYPSDISVEWEKNGKA
EDNYKTTPAVLDSDGSYFLYNKLSVPTSEWQRGDVFTCSVMHEALHNHY
TQKSISRSPGK,

and the anti-CD83 scFv V_{L} domain comprises the amino acid sequence:

(SEQ ID NO: 24, 11G05)
MDTREPTQLLGLLLLWLPGARCADVVMTQTPASVSAAVGGTVTINCQSS
KNVYNNNWLSWFQQKPGQPPKLLIYYASTLASGVPSRFRGSGSGTQFTL
TISDVQCDDAATYYCAGDYSSSSDNGFGGGTEVWVKGDPVAPTVLLFPP
SSDEVATGTVTIVCVANKYFPDVTVTWEVDGTTQTTGIENSKTPQNSAD
CTYNLSSTLTLTSTQYNSHKEYTCKVTQGTTSVVQSFSRKNC.

In some embodiments, the anti-CD83 scFv V_{H} domain comprises the amino acid sequence:

(SEQ ID NO: 25, 14C12)
METGLRWLLLVAVLKGVHCQSVEESGGRLVTPGTPLTLTCTASGFSRSS
YDMSWVRQAPGKGLEWVGVISTAYNSHYASWAKGRFTISRTSTTVDLKM
TSLTTEDTATYFCARGGSWLDLWGQGTLVTVSSGQPKAPSVFPLAPCCG
DTPSSTVTLGCLVKGYLPEPVTVTWNSGTLTNGVRTFPSVRQSSGLYSL
SSVVSVTSSSQPVTCNVAHPATNTKVDKTVAPSTCSKPTCPPPELLGGP
SVFIFPPKPKDTLMISRTPEVTCVVVDVSQDDPEVQFTWYINNEQVRTA
RPPLREQQFNSTIRVVSTLPIAHQDWLRGKEFKCKVHNKALPAPIEKTI
SKARGQPLEPKVYTMGPPREELSSRSVSLTCMINGFYPSDISVEWEKNG
KAEDNYKTTPAVLDSDGSYFLYNKLSVPTSEWQRGDVFTCSVMHEALHN
HYTQKSISRSPGK,

and the anti-CD83 scFv V_{L} domain comprises the amino acid sequence:

(SEQ ID NO: 26, 14C12)
MDXRAPTQLLGLLLLWLPGARCALVMTQTPASVSAAVGGTVTINCQSSQ
SVYDNDELSWYQQKPGQPPKLLIYALASKLASGVPSRFKGSGSGTQFAL
TISGVQCDDAATYYCQATHYSSDWYLTFGGGTEVVVKGFPVAPTVLLFP
PSSDEVATGTVTIVCVANKYFPDVTVTWEVDGTTQTTGTENSKTPQNSA
DCTYNLSSTLTLTSTQYNSHKEYTCKVTQGTTSVVQSFSRKNC.

In some embodiments, the anti-CD83 scFv V_{H} domain comprises the amino acid sequence:

(SEQ ID NO: 27, 020B08)
METGLRWLLLVAVLKGVQCQSVEESGGRLVTPGTPLTLTCTVSGFSLSS
YDMTWVRQAPGKGLEWIGIIYASGTTYYANWAKGRFTISKTSTTVDLKV
TSPTIGDTATYFCAREGAGVSMTLWGPGTLVTVSSGQPKAPSVFPLAPC
CGDTPSSTVTLGCLVKGYLPEPVTVTWNSGTLTNGVRTFPSVRQSSGLY
SLSSVVSVTSSSQPVTCNVAHPATNTKVDKTVAPSTCSKPTCPPPELLG
GPSVFIFPPKPKDTLMISRTPEVTCVVVDVSQDDPEVQFTWYINNEQVR
TARPPLREQQFNSTIRVVSTLPIAHQDWLRGKEFKCKVHNKALPAPIEK
TISKARGQPLEPKVYTMGPPREELSSRSVSLTCMINGFYPSDISVEWEK
NGKAEDNYKTTPAVLDSDGSYFLYNKLSVPTSEWQRGDVFTCSVMHEAL
HNHYTQKSISRSPGK,

and the anti-CD83 scFv V_{L} domain comprises the amino acid sequence:

(SEQ ID NO: 28,020B08)
MDMRAPTQLLGLLLLWLPGARCAYDMTQTPASVEVAVGGTVTIKCQASQ
SISTYLDWYQQKPGQPPKLLIYDASDLASGVPSRFKGSGSGTQFTLTIS
DLECADAATYYCQQGYTHSNVDNVFGGGTEVVVKGDPVAPTVLLFPPSS
DEVATGTVTIVCVANKYFPDVTVTWEVDGTTQTTGIENSKTPQNSADCT
YNLSSTLTLTSTQYNSHKEYTCKVTQGTTSVVQSFSRKNC

In some embodiments, the anti-CD83 scFv V_{H} domain comprises the amino acid sequence:

(SEQ ID NO: 29, 006G05)
METGLRWLLLVAVLKGVQCQSVEESGGRLVSPGTPLTLTCTASGFSLSS
YDMSWVRQAPGKGLEYIGIISSSGSTYYASWAKGRFTISKTSTTVDLEV
TSLTTEDTATYFCSREHAGYSGDTGHLWGPGTLVTVSSGQPKAPSVFPL
APCCGDTPSSTVTLGCLVKGYLPEPVTVTWNSGTLTNGVRTFPSVRQSS
GLYSLSSVVSVTSSSQPVTCNVAHPATNTKVDKTVAPSTCSKPTCPPPE
LLGGPSVGIGPPKPKDTLMISRTPEVTCVVVDVSQDDPEVQFTWYINNE
QVRTARPPLREQQFNSTIRVVSTLPIAHQDWLRGKEFKCKVHNKALPAP
IEKTISKARGQPLEPKVYTMGPPREELSSRSVSLTCMINGFYPSDISVE
WEKNGKAEDNYKTTPAVLDSDGSYFLYNKLSVPTSEWQRGDVFTCSVMH
EALHNHYTQKSISRSPGK,

and the anti-CD83 scFv V_{L} domain comprises the amino acid sequence:

(SEQ ID NO: 30, 006G05)
MDMRAPTQLLGLLLLWLPGARCAYDMTQTPASVEVAVGGTVAIKCQASQ
SVSSYLAWYQQKPGQPPKPLIYEASMLAAGVSSRFKGSGSGTDFTLTIS
DLECDDAATYYCQQGYSISDIDNAFGGGTEVVVKGDPVAPTVLLFPPSS
DEVATGTVTIVCVANKYFPDVTVTWEVDGTTQTTGIENSKTPQNSADCT
YNLSSTLTLTSTQYNSHKEYTCKVTQGTTSVVQSFSRKNC

In some embodiments, the anti-CD83 scFv V_{H} domain comprises the amino acid sequence:

(SEQ ID NO: 31, 96G08)
METGLRWLLLVAVLKGVQCQSVEESGGRLVTPGTPLTLTCTVSGIDLSS
DGISWVRQAPGKGLEWIGIISSGGNTYYASWAKGRFTISRTSTTVDLKM
TSLTTEDTATYFCARVVGGTYSIWGQGTLVTVSSASTKGPSVYPLAPGS
AAQTNSMVTLGCLVKGYFPEPVTVTWNSGSLSSGVHTFPAVLQSDLYTL
SSSVTVPSSTWPSETVTCNVAHPASSTKVDKKIVPRDCGCKPCICTVPE
VSSVFIFPPKPDVLTITLTPKVTCVVVDISKDDPEVQFSWFVDDVEVHT
AQTQPREEQFNSTFRSVSELPIMHQDWLNGKEFKCRVNSAAFPAPIEKT
ISKTKGRPKAPQVYTIPPPKEQMAKDKVSLTCMITDFFPEDITVEWQWN
GQPAENYKNTQPIMDTDGSYFVYSKLNVQKSNWEAGNTFTCSVLHEGLH
NHHTEKSLSHSPGK,

and the anti-CD83 scFv V_{L} domain comprises the amino acid sequence:

(SEQ ID NO: 32, 96G08)
MDTRAPTQLLGLLLLWLPGATFAQVLTQTASPVSAPVGGTVTINCQSSQ
SVYNNDFLSWYQQKPGQPPKLLIYYASTLASGVPSRFKGSGSGTQFTLT
ISDLECDDAATYYCTGTYGNSAWYEDAFGGGTEVWVKRTPVAPTVLLFP
PSSAELATGTATIVCVANKYFPDGTVTWKVDGITQSSGINNSRTPQNSA
DCTYNLSSTLTLSSDEYNSHDEYTCQVAQDSGSPVVQSFSRKSC

In some embodiments, the anti-CD83 scFv V_{H} domain comprises the amino acid sequence:

(SEQ ID NO: 33, 95F04)
METGLRWLLLVAVLKGVQCQSVEESGGRLVTPGTPLTLTCTVSGIDLSS
NAMIWVRQAPREGLEWIGAMDSNSRTYYATWAKGRFTISRTSSITVDLK
ITSPTTEDTATYFCARGDGGSSDYTEMWGPGTLVTVSSASTKGPSVYPL
APGSAAQTNSMVTLGCLVKGYFPEPVTVTWNSGSLSSGVHTFPAVLQSD
LYTLSSSVTVPSSTWPSETVTCNVAHPASSTKVDKKIVPRDCGCKPCIC
TVPEVSSVFIFPPKPKDVLTITLTPKVTCVVVDISKDDPEVQFSWFVDD
VEVHTAQTQPREEQFNSTFRSVSELPIMHQDWLNGKEFKCRVNSAAFPA
PIEKTISKTKGRPKAPQVYTIPPPKEQMAKDKVSLTCMITDFFPEDITV
EWQWNGQPAENYKNTQPIMDTDGSYFVYSKLNVQKSNWEAGNTFTCSVL
HEGLHNHHTEKSLSHSPGK,

and the anti-CD83 scFv V_{L} domain comprises the amino acid sequence:

(SEQ ID NO: 34, 95F04)
MDTRAPTQLLGLLLLWLPGATFAQAVVTQTTSPVSAPVGGTVTINCQSS
QSVYGNNELSWYQQKPGQPPKLLIYQASSLASGVPSRFKGSGSGTQFTL
TISDLECDDAATYYCLGEYSISADNHFGGGTEVVVKRTPVAPTVLLFPP
SSAELATGTATIVCVANKYFPDGTVTWKVDGITQSSGINNSRTPQNSAD
CTYNLSSTLTLSSDEYNSHDEYTCQVAQDSGSPVVQSFSRKSC

In some embodiments, the anti-CD83 scFv V_{H} domain comprises the amino acid sequence:

(SEQ ID NO: 35)
QVQLVQSGGAVVQPGRSLRLSCAASGFTFSTYGMHWVRQAPGKGLEWVA
AVSYDGSNKYYADFVKGRFTISRDNPKNTLYLQMNSLRADDTAVYYCAR
RGGLDIWGQGTTVTVSSASTKGPSVFPLAPSSKSTSGGTAALGCLVKDY
FPEPVTVSWNSGALTSGVHTFPAVLQSSGLYSLSSVVTVPSSSLGTQTY
ICNVNHKPSNTKVDKKVEPKSCAAA.

In some embodiments, the anti-CD83 scFv V_{L} domain comprises the amino acid sequence:

(SEQ ID NO: 36)
LTQPPPASGTPGQQRVTISCSGSSSNIGSNTVNWYQQLPGTAPKLLIYY
GNDQRPSGVPDRFSASKSGTSASLAISGLQSEDEAHYYCAAWDGSLNGG
VIFGGGTKVTLG.

In some embodiments, the anti-CD83 scFv V_{L} domain comprises the amino acid sequence:

(SEQ ID NO: 37)
VTQPPSASGTPGQRVTISCSGSSSNIGTNPVNWYQQLPGTAPKLLIYTT
DQRPSGVPDRFSGSKSGTSASLAISGLQSEDEADYYCAAWDDSLSGLYV
FGTGTKVTVLG.

In some embodiments, the anti-CD83 scFv VL domain comprises the amino acid sequence:

(SEQ ID NO: 38)
MTHTPLSLSVTPGQPASISCKSSQSLLHSDGKTYLYWYLQRPGQSPQPL
IYEVSNRFSGVPDRFSGSGSGTDFTLKISRVQAEDVGVYYCMQSLQLWT
FGQGTKVEIKR.

In some embodiments, the anti-CD83 scFv V_{L} domain comprises the amino acid sequence:

(SEQ ID NO: 39)
MTQSPLSLPVTLGQPASISCRSSQSLIHSDGNTYLDWFQQRPGQSPRRL
IYKVSNRDSGVPDRFSGSGSGTDFTLRISRVEAEDIGVYYCMQATHWPR
TFGQGTKVEIKR.

In some embodiments, the anti-CD83 scFv V_{L} domain comprises the amino acid sequence:

(SEQ ID NO: 40)
MTQSPLSLPVTLGQPASISCRSSQSLVDSAGNTFLHWFHQRPGQSPRRL
IYKVSNRDSGVPDRFSGSGSGTDFTLKISRVEAEDVGVYYCMQGTHWPR
TFGQGTKVEIKR.

In some embodiments, the anti-CD83 scFv V_{L} domain comprises the amino acid sequence:

(SEQ ID NO: 41)
LTQSPLSLPVTLGQPASISCKSSQSLVDSDGNTYLNWFQQRPGQSPRRL
IYKVSNRDSGVPDRFSGSGSGTDFTLKISRVEAEDVGVYYCMQGTHWPR
TFGQGTKVEIKR.

In some embodiments, the anti-CD83 scFv V_{L} domain comprises the amino acid sequence:

(SEQ ID NO: 42)
MTQSPLSLPVTLGQPASISCRSSQSLVHSDGNMYLNWFQQRPGQSPRRLI
YKVSNRDSGVPDRFSGSGSGTDFTLKISRVEAEDVGVYYCMQATQPTWTF
GQGTKLEIKR.

In some embodiments, the anti-CD83 scFv V_{L} domain comprises the amino acid sequence:

(SEQ ID NO: 43)
MTQSPSSLSASVGDRVTITCQASQDISNYLNWYQQKPGKAPKLLIYDASN
LETGVPSRFSGSGSGTDFTFTISSATYYCQQTYQGTKLEIKR.

In some embodiments, the anti-CD83 scFv V_{L} domain comprises the amino acid sequence:

(SEQ ID NO: 44)
MTQSPSSLSASVGHPVTITCRASQSLISYLNWYHQKPGKAPKLLIYAASI
LQSGVPSRFSGSGSGTDFTLTISSLQPENFASYYCQHTDSFPRTFGHGTK
VEIKR.

In some embodiments, the anti-CD83 scFv V_{L} domain comprises the amino acid sequence:

(SEQ ID NO: 45)
LTQPPSASGTPGQGVTISCRGSTSNIGNNVVNWYQHVPGSAPKLLIWSNI
QRPSGIPDRFSGSKSGTSASLAISGLQSEDQAVYYCAVWDDGLAGWVFGG
GTTVTVLS.

In some embodiments, the anti-CD83 scFv V_{L} domain comprises the amino acid sequence:

(SEQ ID NO: 46)
MTQAPVVSVALEQTVRITCQGDSLAIYYDFWYQHKPGQAPVLVIYGKNNR
PSGIPHRFSGSSSNTDSLTITGAQAEDEADYYCNSRDSSGNHWVFGGGTN
LTVLG.

In some embodiments, the anti-CD83 scFv V_{L} domain comprises the amino acid sequence:

(SEQ ID NO: 47)
LTQSPLSLPVTLGQPASISCKSNQSLVHSDGNTYLNWFQQRPGQSPRRLI
YKVSNRDSGVPDRFSGSGSGTDFTLKINRVEAEDVGVYYCMQGTQWPRTF
GGQGTKLDIKR.

In some embodiments, the anti-CD83 scFv V_{H} domain has been humanized and comprises the amino acid sequence:

(SEQ ID NO: 48, VH-GBM01)
QVQLQESGPGLVKPSETLSLTCTVSGFSITTGGYWWTWIRQPPGKGLEWI
GYIFSSGNTNYNPSIKSRVTISVDTSKNQFSLKLSSVTAADTAVYYCARA
YGKLGFDYWGQGTLVTVSS,
(SEQ ID NO: 49, VH-GBM02)
QVQLQESGPGLVKPSQTLSLTCTVSGFSITTGGYWWTWIRQHPGKGLEWI
GYIFSSGNTNYNPSIKSLVTISVDTSKNQFSLKLSSVTAADTAVYYCARA
YGKLGFDYWGQGTLVTVSS,
(SEQ ID NO: 50, VH-GBM03)
QVQLQESGPGLVKPSQTLSLTCTVSGFSITTGGYWWTWIRQPPGKGLEWI
GYIFSSGNTNYNPSIKSRVTISVDTSKNQFSLKLSSVTAADTAVYYCARA
YGKLGFDYWGQGTLVTVSS,
(SEQ ID NO: 51, VH-GBM04)
QVQLQESGPGLVKPSETLSLTCTVSGFSITTGGYWWTWIRQPPGKGLEWI
GYIFSSGNTNYNPSIKSRVTISRDTSKNQFSLKLSSVTAADTAVYYCARA
YGKLGFDYWGQGTLVTVSS,
(SEQ ID NO: 52, VH-GBM05)
QVQLQESGPGLVKPSETLSLTCTVSGFSITTGGYWWTWIRQPPGKGLEWI
GYIFSSGNTNYNPSIKSRVTISVDTSKNQFSLKLSSVTAADTARYYCARA
YGKLGFDYWGQGTLVTVSS,
or
(SEQ ID NO: 53, VH-GBM06)
QVQLQESGPGLVKPSETLSLTCTVSGFSITTGGYWWTWIRQPPGKGLEWI
GYIFSSGNTNYNPSIKSRISITRDTSKNQFFLQLNSVTTEGDTARYYCAR
AYGKLGFDYWGQGTLVTVSS;

and the anti-CD83 scFv V_{L} domain has been humanized and comprises the amino acid sequence:

(SEQ ID NO: 54, VL-GBM01)
QLVLTQSPSASASLGASVKLTCTLSSQHSTYTIGWHQQQPEKGPRYLMKV
NSDGSHSKGDGIPDRFSGSSSGAERYLTISSLQSEDEADYYCGSSDSSGY
VFGSGTKVTVL,
or
(SEQ ID NO: 55, VL-GBM02)
LPVLTQPPSASALLGASIKLTCTLSSQHSTYTIGWYQQRPGRSPQYIMKV
NSDGSHSKGDGIPDRFMGSSSGADRYLTFSNLQSDDEAEYHCGSSDSSGY
VFGSGTKVTVL.

The heavy and light chains are preferably separated by a linker. Suitable linkers for scFv antibodies are known in the art. In some embodiments, the linker comprises the amino acid sequence GGGGSGGGGSGGGGS (SEQ ID NO:56).

In some embodiments, the anti-CD83 scFv comprises an amino acid sequence:

(SEQ ID NO: 57)
QPVLTQSPSASASLGNSVKITCTLSSQHSTYTIGWYQQHPDKAPKYVMYV
NSDGSHSKGDGIPDRFSGSSSGAHRYLSISNIQPEDEADYFCGSSDSSGY
VFGSGTQLTVLRAAASSGGGGSGGGGSGGGGSQPVLTQSPSASASLGNSV
KITCTLSSQHSTYTIGWYQQHPDKAPKYVMYVNSDGSHSKGDGIPDRFSG
SSSGAHRYLSISNIQPEDEADYFCGSSDSSGYVFGSGTQLTVLRAAA.

In some embodiments, the anti-CD83 scFv comprises an amino acid sequence:

(SEQ ID NO: 58)
QVQLKESGPGLVKPSQSLSLTCSVTGFSITTGGYWWTWIRQFPGQKLEWM
GYIFSSGNTNYNPSIKSRISITRDTSKNQFFLQLNSVTTEGDTARYYCAR
AYGKLGFDYWGQGTLVTVSSGGGGSGGGGSGGGGSQVQLKESGPGLVKPS
QSLSLTCSVTGFSITTGGYWWTWIRQFPGQKLEWMGYIFSSGNTNYNPSI
KSRISITRDTSKNQFFLQLNSVTTEGDTARYYCARAYGKLGFDYWGQGTL
VTV.

In some embodiments, the anti-CD83 scFv comprises an amino acid sequence:

(SEQ ID NO: 59)
QVQLQESGPGLVKPSETLSLTCTVSGFSITTGGYWWTWIRQPPGKGLEWI
GYIFSSGNTNYNPSIKSRVTISVDTSKNQFSLKLSSVTAADTAVYYCARA
YGKLGFDYWGQGTLVTVSSGGGGSGGGGSGGGGSQLVLTQSPSASASLGA
SVKLTCTLSSQHSTYTIGWHQQQPEKGPRYLMKVNSDGSHSKGDGIPDRF
SGSSSGAERYLTISSLQSEDEADYYCGSSDSSGYVFGSGTKVTVL.

In some embodiments, the anti-CD83 scFv comprises an amino acid sequence:

(SEQ ID NO: 60)
QVQLQESGPGLVKPSQTLSLTCTVSGFSITTGGYWWTWIRQHPGKGLEWI
GYIFSSGNTNYNPSIKSLVTISVDTSKNQFSLKLSSVTAADTAVYYCARA
YGKLGFDYWGQGTLVTVSSGGGGSGGGGSGGGGSQLVLTQSPSASASLGA
SVKLTCTLSSQHSTYTIGWHQQQPEKGPRYLMKVNSDGSHSKGDGIPDRF
SGSSSGAERYLTISSLQSEDEADYYCGSSDSSGYVFGSGTKVTVL.

In some embodiments, the anti-CD83 scFv comprises an amino acid sequence:

(SEQ ID NO: 61)
QVQLQESGPGLVKPSQTLSLTCTVSGFSITTGGYWWTWIRQPPGKGLEWI
GYIFSSGNTNYNPSIKSRVTISVDTSKNQFSLKLSSVTAADTAVYYCARA
YGKLGFDYWGQGTLVTVSSGGGGSGGGGSGGGGSQLVLTQSPSASASLGA
SVKLTCTLSSQHSTYTIGWHQQQPEKGPRYLMKVNSDGSHSKGDGIPDRF
SGSSSGAERYLTISSLQSEDEADYYCGSSDSSGYVFGSGTKVTVL.

In some embodiments, the anti-CD83 scFv comprises an amino acid sequence:

(SEQ ID NO: 62)
QVQLQESGPGLVKPSETLSLTCTVSGFSITTGGYWWTWIRQPPGKGLEWI
GYIFSSGNTNYNPSIKSRVTISRDTSKNQFSLKLSSVTAADTAVYYCARA
YGKLGFDYWGQGTLVTVSSGGGGSGGGGSGGGGSQLVLTQSPSASASLGA
SVKLTCTLSSQHSTYTIGWHQQQPEKGPRYLMKVNSDGSHSKGDGIPDRF
SGSSSGAERYLTISSLQSEDEADYYCGSSDSSGYVFGSGTKVTVL.

In some embodiments, the anti-CD83 scFv comprises an amino acid sequence:

(SEQ ID NO: 63)
QVQLQESGPGLVKPSETLSLTCTVSGFSITTGGYWWTWIRQPPGKGLEWI
GYIFSSGNTNYNPSIKSRVTISVDTSKNQFSLKLSSVTAADTARYYCARA
YGKLGFDYWGQGTLVTVSSGGGGSGGGGSGGGGSQLVLTQSPSASASLGA
SVKLTCTLSSQHSTYTIGWHQQQPEKGPRYLMKVNSDGSHSKGDGIPDRF
SGSSSGAERYLTISSLQSEDEADYYCGSSDSSGYVFGSGTKVTVL.

In some embodiments, the anti-CD83 scFv comprises an amino acid sequence:

(SEQ ID NO: 64)
QVQLQESGPGLVKPSETLSLTCTVSGFSITTGGYWWTWIRQPPGKGLEW
IGYIFSSGNTNYNPSIKSRISITRDTSKNQFFLQLNSVTTEGDTARYYC
ARAYGKLGFDYWGQGTLVTVSSGGGGSGGGGSGGGGSQLVLTQSPSASA
SLGASVKLTCTLSSQHSTYTIGWHQQQPEKGPRYLMKVNSDGSHSKGDG
IPDRFSGSSSGAERYLTISSLQSEDEADYYCGSSDSSGYVFGSGTKVTV
L.

In some embodiments, the anti-CD83 scFv comprises an amino acid sequence:

(SEQ ID NO: 65)
QVQLQESGPGLVKPSETLSLTCTVSGFSITTGGYWWTWIRQPPGKGLEW
IGYIFSSGNTNYNPSIKSRVTISVDTSKNQFSLKLSSVTAADTAVYYCA
RAYGKLGFDYWGQGTLVTVSSGGGGSGGGGSGGGGSLPVLTQPPSASAL
LGASIKLTCTLSSQHSTYTIGWYQQRPGRSPQYIMKVNSDGSHSKGDGI
PDRFMGSSSGADRYLTFSNLQSDDEAEYHCGSSDSSGYVFGSGTKVTV
L.

In some embodiments, the anti-CD83 scFv comprises an amino acid sequence:

(SEQ ID NO: 66)
QVQLQESGPGLVKPSQTLSLTCTVSGFSITTGGYWWTWIRQHPGKGLEW
IGYIFSSGNTNYNPSIKSLVTISVDTSKNQFSLKLSSVTAADTAVYYCA
RAYGKLGFDYWGQGTLVTVSSGGGGSGGGGSGGGGSLPVLTQPPSASAL
LGASIKLTCTLSSQHSTYTIGWYQQRPGRSPQYIMKVNSDGSHSKGDGI
PDRFMGSSSGADRYLTFSNLQSDDEAEYHCGSSDSSGYVFGSGTKVTV
L.

In some embodiments, the anti-CD83 scFv comprises an amino acid sequence:

(SEQ ID NO: 67)
QVQLQESGPGLVKPSQTLSLTCTVSGFSITTGGYWWTWIRQPPGKGLEW
IGYIFSSGNTNYNPSIKSRVTISVDTSKNQFSLKLSSVTAADTAVYYCA
RAYGKLGFDYWGQGTLVTVSSGGGGSGGGGSGGGGSLPVLTQPPSASAL
LGASIKLTCTLSSQHSTYTIGWYQQRPGRSPQYIMKVNSDGSHSKGDGI
PDRFMGSSSGADRYLTFSNLQSDDEAEYHCGSSDSSGYVFGSGTKVTV
L.

In some embodiments, the anti-CD83 scFv comprises an amino acid sequence:

(SEQ ID NO: 68)
QVQLQESGPGLVKPSETLSLTCTVSGFSITTGGYWWTWIRQPPGKGLEW
IGYIFSSGNTNYNPSIKSRVTISRDTSKNQFSLKLSSVTAADTAVYYCA
RAYGKLGFDYWGQGTLVTVSSGGGGSGGGGSGGGGSLPVLTQPPSASAL
LGASIKLTCTLSSQHSTYTIGWYQQRPGRSPQYIMKVNSDGSHSKGDGI
PDRFMGSSSGADRYLTFSNLQSDDEAEYHCGSSDSSGYVFGSGTKVTV
L.

In some embodiments, the anti-CD83 scFv comprises an amino acid sequence:

(SEQ ID NO: 69)
QVQLQESGPGLVKPSETLSLTCTVSGFSITTGGYWWTWIRQPPGKGLEW
IGYIFSSGNTNYNPSIKSRVTISVDTSKNQFSLKLSSVTAADTARYYCA
RAYGKLGFDYWGQGTLVTVSSGGGGSGGGGSGGGGSLPVLTQPPSASAL
LGASIKLTCTLSSQHSTYTIGWYQQRPGRSPQYIMKVNSDGSHSKGDGI
PDRFMGSSSGADRYLTFSNLQSDDEAEYHCGSSDSSGYVFGSGTKVTV
L.

In some embodiments, the anti-CD83 scFv comprises an amino acid sequence:

(SEQ ID NO: 70)
QVQLQESGPGLVKPSETLSLTCTVSGFSITTGGYWWTWIRQPPGKGLEW
IGYIFSSGNTNYNPSIKSRISITRDTSKNQFFLQLNSVTTEGDTARYYC
ARAYGKLGFDYWGQGTLVTVSSGGGGSGGGGSGGGGSLPVLTQPPSASA
LLGASIKLTCTLSSQHSTYTIGWYQQRPGRSPQYIMKVNSDGSHSKGDG
IPDRFMGSSSGADRYLTFSNLQSDDEAEYHCGSSDSSGYVFGSGTKVTV
L.

In some embodiments, the anti-CD83 scFv comprises an amino acid sequence:

(SEQ ID NO: 71)
QVQLKESGPGLVKPSQSLSLTCSVTGFSITTGGYWWTWIRQFPGQKLEW
MGYIFSSGNTNYNPSIKSRISITRDTSKNQFFLQLNSVTTEGDTARYYC
ARAYGKLGFDYWGQGTLVTVSSGGGGSGGGGSGGGGSQPVLTQSPSASA
SLGNSVKITCTLSSQHSTYTIGWYQQHPDKAPKYVMYVNSDGSHSKGDG
IPDRFSGSSSGAHRYLSISNIQPEDEADYFCGSSDSSGYVFGSGTQLTV
L.

### Anti-CD33

In some embodiments, the anti-CD33 scFv can comprise a variable heavy (V_{H}) domain having CDR1, CDR2 and CDR3 sequences and a variable light (V_{L}) domain having CDR1, CDR2 and CDR3 sequences.

For example, in some embodiments, the CDR1 sequence of the V_{H} domain comprises the amino acid sequence GFTFSNYG (SEQ ID NO:72), the CDR2 sequence of the V_{H} domain comprises the amino acid sequence ISSGGGDT (SEQ ID NO:73), the CDR3 sequence of the V_{H} domain comprises the amino acid sequence ARDYGGTWDYFDY (SEQ ID NO:74); or
the CDR1 sequence of the V_{H} domain comprises the amino acid sequence GYTFTSYW (SEQ ID NO:75), the CDR2 sequence of the V_{H} domain comprises the amino acid sequence IHPSDSET (SEQ ID NO:76), the CDR3 sequence of the V_{H} domain comprises the amino acid sequence AREEGQLGHGGAMDY (SEQ ID NO:77); and
the CDR1 sequence of the V_{L} comprises the amino acid sequence QDISKY (SEQ ID NO:78), wherein the CDR2 sequence of the V_{L} domain comprises the amino acid sequence YTS, wherein the CDR3 sequence of the V_{L} domain comprises the amino acid sequence QQGDTFPWT (SEQ ID NO:80).

Therefore, in some embodiments, the anti-CD33 scFv V_{H} domain comprises the amino acid sequence

(SEQID NO: 81, 6A11HC1)
EVKLVESGGGLVKPGASLKLSCAASGFTFSNYGMSWVRQTSDKRL
EWVASISSGGGDTYYPDNVKGRFTISRENAKNTLYLQMSSLNSED
TALYYCARDYGGTWDYFDYWGQGTTLTVSS,
or
(SEQ ID NO: 82, 6A11HC2)
QVQLQQPGAELVRPGVSVKLSCKASGYTFTSYWMNWVKQRPGQGL
EWIGMIHPSDSETRLNQKFKDKAILTVDKSSSTAYMQLSSPTSED
SAVYYCAREEGQLGHGGAMDYWGQGTSVTVSS;

and
the anti-CD33 scFv V_{L} domain comprises the amino acid sequence

(SEQ ID NO: 83, 6A11LC)
DIQMTQTTSSLSASLGDRVTISCRASQDISKYLNWYQQKPDGTVK
LLIYYTSRLHSGVPSRFSGSGSGTDYSLTISNLEQEDIATYFCQQ
GDTFPWTFGGGTKLEIK.

Therefore, in some embodiments, the anti-CD33 scFv V_{H} domain comprises the amino acid sequence

(SEQ ID NO: 84, 27A3HC)
QVQLKESGPGLVAPSQSLSITCTVSGFSLSRYSVHWVRQPPGKGL
EWLGMIWGGGYTDYNSALKSRLSISKDNSKSQVFLKMNSLQTDDT
AMYYCARYIDSSGYDYWGQGTTLTVSS;

and the anti-CD83 scFv V_{L} domain comprises the amino acid sequence

(SEQ ID NO: 85, 27A3LC1)
SIVMTQTPKFLLVSAGDRVTITCKASQTVNDDVAWYQQKPGQSPK
LLIYYVSNRHTGVPDRFTGSGYGTDFTFTISTVQAEDLAVYFCQQ
DYSSPYTFGGGTKLEIK,
(SEQ ID NO: 86, 27A3LC2)
QIVLTQSPAIMSASPGEKVTMTCSASSSVSYMHWYQQKSGTSPKR
WIYDTSKLASGVPARFSGSGSGTSYSLTISSMEAEDAATYYCQQW
SSNPLTFGAGTKLELK,
or
(SEQ ID NO: 87, 27A3LC3)
DIQMTQSPASLSASVGETVTITCRASENIYSYLAWYQQKQGKSPQ
LLVYNAKTLAEGVPSRFSGSGSGTQFSLKINSLQPEDFGSYYCQH
HYGTPYTFGGGTKLEIK.

In some embodiments, the anti-CD33 scFv comprises the amino acid sequence:

(SEQ ID NO: 88, 6A11HC1_LC)
EVKLVESGGGLVKPGASLKLSCAASGFTFSNYGMSWVRQTSDKRLEWVAS
ISSGGGDTYYPDNVKGRFTISRENAKNTLYLQMSSLNSEDTALYYCARDY
GGTWDYFDYWGQGTTLTVSSGGGGSGGGGSGGGGSDIQMTQTTSSLSASL
GDRVTISCRASQDISKYLNWYQQKPDGTVKLLIYYTSRLHSGVPSRFSGS
GSGTDYSLTISNLEQEDIATYFCQQGDTFPWTFGGGTKLEIK.

In some embodiments, the anti-CD33 scFv comprises the amino acid sequence:

(SEQ ID NO: 89, 6A11HC2_LC)
QVQLQQPGAELVRPGVSVKLSCKASGYTFTSYWMNWVKQRPGQGLEWIG
MIHPSDSETRLNQKFKDKAILTVDKSSSTAYMQLSSPTSEDSAVYYCAR
EEGQLGHGGAMDYWGQGTSVTVSSGGGGSGGGGSGGGGSDIQMTQTTSS
LSASLGDRVTISCRASQDISKYLNWYQQKPDGTVKLLIYYTSRLHSGVP
SRFSGSGSGTDYSLTISNLEQEDIATYFCQQGDTFPWTFGGGTKLEIK.

In some embodiments, the anti-CD33 scFv comprises the amino acid sequence:

(SEQ ID NO: 90, 27A3HC_LC1)
QVQLKESGPGLVAPSQSLSITCTVSGFSLSRYSVHWVRQPPGKGLEWLG
MIWGGGYTDYNSALKSRLSISKDNSKSQVFLKMNSLQTDDTAMYYCARY
IDSSGYDYWGQGTTLTVSSGGGGSGGGGSGGGGSSIVMTQTPKFLLVSA
GDRVTITCKASQTVNDDVAWYQQKPGQSPKLLIYYVSNRHTGVPDRFTG
SGYGTDFTFTISTVQAEDLAVYFCQQDYSSPYTFGGGTKLEIK.

In some embodiments, the anti-CD33 scFv comprises the amino acid sequence:

(SEQ ID NO: 91, 27A3HC_LC2)
QVQLKESGPGLVAPSQSLSITCTVSGFSLSRYSVHWVRQPPGKGLEWLG
MIWGGGYTDYNSALKSRLSISKDNSKSQVFLKMNSLQTDDTAMYYCARY
IDSSGYDYWGQGTTLTVSSGGGGSGGGGSGGGGSQIVLTQSPAIMSASP
GEKVTMTCSASSSVSYMHWYQQKSGTSPKRWIYDTSKLASGVPARFSGS
GSGTSYSLTISSMEAEDAATYYCQQWSSNPLTFGAGTKLELK.

In some embodiments, the anti-CD33 scFv comprises the amino acid sequence:

(SEQ ID NO: 92, 27A3HC_LC3)
QVQLKESGPGLVAPSQSLSITCTVSGFSLSRYSVHWVRQPPGKGLEWLG
MIWGGGYTDYNSALKSRLSISKDNSKSQVFLKMNSLQTDDTAMYYCARY
IDSSGYDYWGQGTTLTVSSGGGGSGGGGSGGGGSDIQMTQSPASLSASV
GETVTITCRASENIYSYLAWYQQKQGKSPQLLVYNAKTLAEGVPSRFSG
SGSGTQFSLKINSLQPEDFGSYYCQHHYGTPYTFGGGTKLEIK.

### Anti-CLEC12A

In some embodiments, the anti-CLEC12A scFv can comprise a variable heavy (V_{H}) domain having CDR1, CDR2 and CDR3 sequences and a variable light (V_{L}) domain having CDR1, CDR2 and CDR3 sequences.

For example, in some embodiments, the CDR1 sequence of the V_{H} domain comprises the amino acid sequence GFTFSSFA (SEQ ID NO:93) or SFAVS (SEQ ID NO:94), CDR2 sequence of the V_{H} domain comprises the amino acid sequence ISSGGAYT (SEQ ID NO:95), CDR3 sequence of the V_{H} domain comprises the amino acid sequence HSGYDGYYLYAMDY (SEQ ID NO:96); or
the CDR1 sequence of the V_{H} domain comprises the amino acid sequence SHDMS (SEQ ID NO:97); the CDR2 sequence of the V_{H} domain comprises the amino acid sequence YISGGGTNIYYSDTVKGRFT (SEQ ID NO:98); the CDR3 sequence of the V_{H} domain comprises the amino acid sequence PNYNYGGSWFAY (SEQ ID NO:99); and
CDR1 sequence of the V_{L} comprises the amino acid sequence SSVHY (SEQ ID NO:100), CDR2 sequence of the V_{L} domain comprises the amino acid sequence DTS, and CDR3 sequence of the V_{L} domain comprises the amino acid sequence QQWTSNPPT (SEQ ID NO:102).

For example, in some embodiments, the anti-CLEC12A V_{H} domain comprises the amino acid sequence

(SEQ ID NO: 103, 1F3H8)
ELILVESGGGLVKPGGSLKLSCAVSGFTFSSFAMSWVRQTPEKRL
EWVATISSGGAYTFYKDSVKGRFTISRDNAKNTLYLQMSSLRSED
SAMYYCARHSGYDGYYLYAMDYWGQGTSVTVSS,
(SEQ ID NO: 104, 1F3A10)
GVQCELILVESGGGLVKPGGSLKLSCAVSGFTFSSFAVSWVRQTP
EKRLEWVATISSGGAYTFYKDSVKGRFTISRDNAKNTLYLQMSSL
RSEDSAMYYCARHSGYDGYYLYAMDYWGQGTSVTVSS,
or
(SEQ ID NO: 105, 1F3F3)
EVQLEESGGGLVQPGGSLKVSCAVSGLAFSSHDMSWVRQTPEKRL
EWVAYISGGGTNIYYSDTVKGRFTISRDNAKNTLYLQMSSLKSED
TAIYYCARPNYNYGGSWFAYWGQGTLVTVSA;

and the anti-CLEC12A V_{L} domain comprises the amino acid sequence

(SEQ ID NO: 106, 1F3H8, 1F3F3, 1F3A10)
QIVLTQSPEIMSASPGEKVTMTCSASSSVHYMHWYQQKSGTSPKR
WIYDTSKLASGVPGRFSGSGSGTSYSLTISSMESEDAATYYCQQW
TSNPPTFGGGTKLEIK.

In some embodiments, the anti-CLEC12A scFv comprises an amino acid sequence:

(SEQ ID NO: 107)
QIVLTQSPEIMSASPGEKVTMTCSASSSVHYMHWYQQKSGTSPKRWIYD
TSKLASGVPGRFSGSGSGTSYSLTISSMESEDAATYYCQQWTSNPPTFG
GGTKLEIKGGGGSGGGGSGGGGSELILVESGGGLVKPGGSLKLSCAVSG
FTFSSFAMSWVRQTPEKRLEWVATISSGGAYTFYKDSVKGRFTISRDNA
KNTLYLQMSSLRSEDSAMYYCARHSGYDGYYLYAMDYWGQGTSVTVSS.

In some embodiments, the anti-CLEC12A scFv comprises an amino acid sequence:

(SEQ ID NO: 108)
QIVLTQSPEIMSASPGEKVTMTCSASSSVHYMHWYQQKSGTSPKRWIYD
TSKLASGVPGRFSGSGSGTSYSLTISSMESEDAATYYCQQWTSNPPTFG
GGTKLEIKGGGGSGGGGSGGGGSGVQCELILVESGGGLVKPGGSLKLSC
AVSGFTFSSFAVSWVRQTPEKRLEWVATISSGGAYTFYKDSVKGRFTIS
RDNAKNTLYLQMSSLRSEDSAMYYCARHSGYDGYYLYAMDYWGQGTSVT
VSS.

In some embodiments, the anti-CLEC12A scFv comprises an amino acid sequence:

(SEQ ID NO: 109)
QIVLTQSPEIMSASPGEKVTMTCSASSSVHYMHWYQQKSGTSPKRWIYD
TSKLASGVPGRFSGSGSGTSYSLTISSMESEDAATYYCQQWTSNPPTFG
GGTKLEIKGGGGSGGGGSGGGGSEVQLEESGGGLVQPGGSLKVSCAVSG
LAFSSHDMSWVRQTPEKRLEWVAYISGGGTNIYYSDTVKGRFTISRDNA
KNTLYLQMSSLKSEDTAIYYCARPNYNYGGSWFAYWGQGTLVTVSA.

### Anti-CD123

In some embodiments, the anti-CD123 scFv can comprise a variable heavy (V_{H}) domain having CDR1, CDR2 and CDR3 sequences and a variable light (V_{L}) domain having CDR1, CDR2 and CDR3 sequences.

For example, in some embodiments, the CDR1 sequence of the V_{H} domain comprises the amino acid sequence GYTFTDYN (SEQ ID NO:110), CDR2 sequence of the V_{H} domain comprises the amino acid sequence INPNNGGT (SEQ ID NO:111), CDR3 sequence of the VA domain comprises the amino acid sequence ARKGYGGNYDYFDY (SEQ ID NO:112), CDR1 sequence of the V_{L} comprises the amino acid sequence QSIGTS (SEQ ID NO:113), CDR2 sequence of the V_{L} domain comprises the amino acid sequence YAS, and CDR3 sequence of the V_{L} domain comprises the amino acid sequence QQSNSWPYT (SEQ ID NO:115).

In some embodiments, the CDR1 sequence of the V_{H} domain comprises the amino acid sequence GFNIKDTY (SEQ ID NO:116) or GFSLSTYGMG (SEQ ID NO:117), the CDR2 sequence of the V_{H} domain comprises the amino acid sequence IDPANGNT (SEQ ID NO:118) or IYWDDDK (SEQ ID NO:119), the CDR3 sequence of the V_{H} domain comprises the amino acid sequence ALYYYGGSLDY (SEQ ID NO:120) or AQSLIYDGYYGFAY (SEQ ID NO:121), the CDR1 sequence of the V_{L} comprises the amino acid sequence QSLLYSGNQKNY (SEQ ID NO:122), the CDR2 sequence of the V_{L} domain comprises the amino acid sequence WAS, and the CDR3 sequence of the V_{L} domain comprises the amino acid sequence QQYYSYPRT (SEQ ID NO:124).

In some embodiments, the CDR1 sequence of the V_{H} domain comprises the amino acid sequence GYTFTYYG (SEQ ID NO:125), the CDR2 sequence of the V_{H} domain comprises the amino acid sequence INTYSGVP (SEQ ID NO:126), the CDR3 sequence of the V_{H} domain comprises the amino acid sequence ARWIYYSDLYGMDY (SEQ ID NO:127), the CDR1 sequence of the V_{L} comprises the amino acid sequence QSIVHSNGDTY (SEQ ID NO:128), the CDR2 sequence of the V_{L} domain comprises the amino acid sequence KVS, and the CDR3 sequence of the V_{L} domain comprises the amino acid sequence (SEQ ID NO:130).

In some embodiments, the CDR1 sequence of the V_{H} domain comprises the amino acid sequence GYTFSSYW (SEQ ID NO:131) or GYTLTTYL (SEQ ID NO:132), the CDR2 sequence of the V_{H} domain comprises the amino acid sequence INPSSGYT (SEQ ID NO:133) or INPNSGSS (SEQ ID NO:134), the CDR3 sequence of the V_{H} domain comprises the amino acid sequence ARDGNYDHWYFDV (SEQ ID NO:135) or AIRHYGGSLFDY (SEQ ID NO:136), the CDR1 sequence of the V_{L} comprises the amino acid sequence QDINSY (SEQ ID NO:137) or QSLLNSRTRKNY (SEQ ID NO:138), the CDR2 sequence of the V_{L} domain comprises the amino acid sequence WAS, or RAN, and the CDR3 sequence of the V_{L} domain comprises the amino acid sequence LQYDELLT (SEQ ID NO:141) or EQSYNLFT (SEQ ID NO:142).

Therefore, in some embodiments, the anti-CD123 scFv V_{H} domain comprises the amino acid sequence:

(SEQ ID NO: 143, 3F5HC1)
EVQLQQSGPELVKPGSSVKISCKASGYTFTDYNMDWVKQSHGKSL
EWIGTINPNNGGTSYNQKFKGKATLTVDKSSSTAYMELRSLTSED
SAVYYCARKGYGGNYDYFDYWGQGTTLTVSS,

and the anti-CD123 scFv V_{L} domain comprises the amino acid sequence:

(SEQ ID NO: 144, 3F5LC1)
DILLTQSPAILSVSPGERVSFSCRASQSIGTSIHWYQQRTNGSPRLLIKY
ASESISGIPSRFSGSGSGTDFTLSINSVESEDIADYYCQQSNSWPYTFGG
GTKLEIK.

Therefore, in some embodiments, the anti-CD123 scFv V_{H} domain comprises the amino acid sequence:

(SEQ ID NO: 145, 12H1HC1)
EVQLQQSGAELVKPGASVKLSCTASGFNIKDTYMHWVKQRPEQGL
EWIGRIDPANGNTIYASKFQGKATITADTSSNTAYMQLSSLTSGD
TAVYYCALYYYGGSLDYWGQGTTLTVSS,
(SEQ ID NO: 146, 12H1HC2)
QVTLKESGPGILQPSQTLSLTCSFSGFSLSTYGMGVSWIRQPSGK
GLEWLAHIYWDDDKRYNPSLKSRLTISKDTSNNQVFLKITSVDTA
DTATYYCAQSLIYDGYYGFAYWGQGTLVTVSA,
or
(SEQ ID NO: 147, 12H2HC1);
QIQLVQSGPELKKPGETVKISCKASGYTFTYYGMNWVKQAPGKGL
EWMGWINTYSGVPTYADDFKGRFAFSLETSVSTAYLQINNLKNED
TATYFCARWIYYSDLYGMDYWGQGTSVTVSS

and the anti-CD123 scFv V_{L} domain comprises the amino acid sequence:

(SEQ ID NO: 148, 12H1LC1)
DIVMSQSPSSLAVSVGERVTMSCKSSQSLLYSGNQKNYLAWYQQK
PGQSPKLLIYWASTRESGVPDRFTGSGSGTDFTLTISSVKAEDLA
VYYCQQYYSYPRTFGGGTKLEIK,
or
(SEQ ID NO: 149, 12H2LC)
DVLMTQSPLSLPVSLGDQASISCRSSQSIVHSNGDTYLEWYLQKP
GQSPKLLIYKVSNRFSGVPDRFSGSGSGTDFTLKISRVEAEDLGV
YHCFQGSHVPWTFGGGTKLEIK.

Therefore, in some embodiments, the anti-CD123 scFv V_{H} domain comprises the amino acid sequence:

(SEQ ID NO: 150, 15A12HC1)
QVQLQQSGAELAKPGASVKMSCKASGYTFSSYWMHWLKQRPGQGL
EWIGYINPSSGYTNYNQKFKDKATLTADKSSSTAYMQLSSLTSED
SAVYYCARDGNYDHWYFDVWGTGTTVTVSS,
or
(SEQ ID NO: 151, 15A12HC2)
QVQLQQPGAELVRPGASVKMSCKASGYTLTTYLMDWVKQRLGQGF
EWIGNINPNSGSSNYNEKFKGKAKLTVDKSSSTAYMQLSSLTSED
SAVYYCAIRHYGGSLFDYWGQGTTLTVSS;

and the anti-CD123 scFv V_{L} domain comprises the amino acid sequence:

(SEQ ID NO: 152, 15A12LC1)
DIKMTQSPSSMYASLGERVTITCKASQDINSYLSWFQQKPGKSPK
TLIYRANRLVDGVPSRFSGSGSGQDYSLTISSLEYEDMGIYYCLQ
YDELLTFGAGTKLELK,
or
(SEQ ID NO: 153, 15A12LC2)
DIVMSQSPSSLAVSAGERVTMSCRSSQSLLNSRTRKNYLAWYQQK
PGQSPKLLIYWASTRESGVPDRFSGSGSGTDFTLTISSVQAEDLA
VYYCEQSYNLFTFGSGTKLEIK.

In some embodiments, the anti-CD123 scFv comprises an amino acid sequence:

(SEQ ID NO: 154, 3F5HC1_LC)
EVQLQQSGPELVKPGSSVKISCKASGYTFTDYNMDWVKQSHGKSLEWIGT
INPNNGGTSYNQKFKGKATLTVDKSSSTAYMELRSLTSEDSAVYYCARKG
YGGNYDYFDYWGQGTTLTVSSGGGGSGGGGSGGGGSDILLTQSPAILSVS
PGERVSFSCRASQSIGTSIHWYQQRTNGSPRLLIKYASESISGIPSRFSG
SGSGTDFTLSINSVESEDIADYYCQQSNSWPYTFGGGTKLEIK.

In some embodiments, the anti-CD123 scFv comprises an amino acid sequence:

(SEQ ID NO: 155, 12H1HC1_LC1)
EVQLQQSGAELVKPGASVKLSCTASGFNIKDTYMHWVKQRPEQGLEWIGR
IDPANGNTIYASKFQGKATITADTSSNTAYMQLSSLTSGDTAVYYCALYY
YGGSLDYWGQGTTLTVSSGGGGSGGGGSGGGGSDIVMSQSPSSLAVSVGE
RVTMSCKSSQSLLYSGNQKNYLAWYQQKPGQSPKLLIYWASTRESGVPDR
FTGSGSGTDFTLTISSVKAEDLAVYYCQQYYSYPRTFGGGTKLEIK.

In some embodiments, the anti-CD123 scFv comprises an amino acid sequence:

(SEQ ID NO: 156, 12H1HC2_LC1)
QVTLKESGPGILQPSQTLSLTCSFSGFSLSTYGMGVSWIRQPSGKGLEWL
AHIYWDDDKRYNPSLKSRLTISKDTSNNQVFLKITSVDTADTATYYCAQS
LIYDGYYGFAYWGQGTLVTVSAGGGGSGGGGSGGGGSDIVMSQSPSSLAV
SVGERVTMSCKSSQSLLYSGNQKNYLAWYQQKPGQSPKLLIYWASTRESG
VPDRFTGSGSGTDFTLTISSVKAEDLAVYYCQQYYSYPRTFGGGTKLEIK.

In some embodiments, the anti-CD123 scFv comprises an amino acid sequence:

(SEQ ID NO: 157, 12H2HC1_LC1)
QIQLVQSGPELKKPGETVKISCKASGYTFTYYGMNWVKQAPGKGLEWMGW
INTYSGVPTYADDFKGRFAFSLETSVSTAYLQINNLKNEDTATYFCARWI
YYSDLYGMDYWGQGTSVTVSSGGGGSGGGGSGGGGSDVLMTQSPLSLPVS
LGDQASISCRSSQSIVHSNGDTYLEWYLQKPGQSPKLLIYKVSNRFSGVP
DRFSGSGSGTDFTLKISRVEAEDLGVYHCFQGSHVPWTFGGGTKLEIK.

In some embodiments, the anti-CD123 scFv comprises an amino acid sequence:

(SEQ ID NO: 158, 15A12HC1_LC1)
QVQLQQSGAELAKPGASVKMSCKASGYTFSSYWMHWLKQRPGQGLEWIGY
INPSSGYTNYNQKFKDKATLTADKSSSTAYMQLSSLTSEDSAVYYCARDG
NYDHWYFDVWGTGTTVTVSSGGGGSGGGGSGGGGSDIKMTQSPSSMYASL
GERVTITCKASQDINSYLSWFQQKPGKSPKTLIYRANRLVDGVPSRFSGS
GSGQDYSLTISSLEYEDMGIYYCLQYDELLTFGAGTKLELK.

In some embodiments, the anti-CD123 scFv comprises an amino acid sequence:

(SEQ ID NO: 159, 15A12HC1_LC2)
QVQLQQSGAELAKPGASVKMSCKASGYTFSSYWMHWLKQRPGQGLEWIGY
INPSSGYTNYNQKFKDKATLTADKSSSTAYMQLSSLTSEDSAVYYCARDG
NYDHWYFDVWGTGTTVTVSSGGGGSGGGGSGGGGSDIVMSQSPSSLAVSA
GERVTMSCRSSQSLLNSRTRKNYLAWYQQKPGQSPKLLIYWASTRESGVP
DRFSGSGSGTDFTLTISSVQAEDLAVYYCEQSYNLFTFGSGTKLEIK.

In some embodiments, the anti-CD123 scFv comprises an amino acid sequence:

(SEQ ID NO: 160, 15A12HC2_LC1)
QVQLQQPGAELVRPGASVKMSCKASGYTLTTYLMDWVKQRLGQGFEWIGN
INPNSGSSNYNEKFKGKAKLTVDKSSSTAYMQLSSLTSEDSAVYYCAIRH
YGGSLFDYWGQGTTLTVSSGGGGSGGGGSGGGGSDIKMTQSPSSMYASLG
ERVTITCKASQDINSYLSWFQQKPGKSPKTLIYRANRLVDGVPSRFSGSG
SGQDYSLTISSLEYEDMGIYYCLQYDELLTFGAGTKLELK.

In some embodiments, the anti-CD123 scFv comprises an amino acid sequence:

(SEQ ID NO: 161, 15A12HC2_LC2)
QVQLQQPGAELVRPGASVKMSCKASGYTLTTYLMDWVKQRLGQGFEWIGN
INPNSGSSNYNEKFKGKAKLTVDKSSSTAYMQLSSLTSEDSAVYYCAIRH
YGGSLFDYWGQGTTLTVSSGGGGSGGGGSGGGGSDIVMSQSPSSLAVSAG
ERVTMSCRSSQSLLNSRTRKNYLAWYQQKPGQSPKLLIYWASTRESGVPD
RFSGSGSGTDFTLTISSVQAEDLAVYYCEQSYNLFTFGSGTKLEIK.

### Anti-CD38

An exemplary anti-CD38 antibody that may be used in the pharmaceutical compositions of the invention is daratumumab.

For example, in some embodiments, the CDR1 sequence of the V_{H} domain comprises the amino acid sequence SFAMS (SEQ ID NO:162), CDR2 sequence of the V_{H} domain comprises the amino acid sequence AISGSGGGTYYADSVKG (SEQ ID NO:163), CDR3 sequence of the V_{H} domain comprises the amino acid sequence DKILWFGEPVFDY (SEQ ID NO:164), CDR1 sequence of the VL comprises the amino acid sequence RASQSVSSYLA (SEQ ID NO:165), CDR2 sequence of the VL domain comprises the amino acid sequence DASNRAT (SEQ ID NO:166), and CDR3 sequence of the VL domain comprises the amino acid sequence QQRSNWPPTF (SEQ ID NO:167).

In some embodiments, the anti-CD38 scFv V_{H} domain has the amino acid sequence:

(SEQ ID NO: 168)
EVQLLESGGGLVQPGGSLRLSCAVSGFTFNSFAMSWVRQAPGKGL
EWVSAISGSGGGTYYADSVKGRFTISRDNSKNTLYLQMNSLRAED
TAVYFCAKDKILWFGEPVFDYWGQGTLVTVSS,

and the anti-CD38 scFv V_{L} domain has the amino acid sequence:

(SEQ ID NO: 169)
EIVLTQSPATLSLSPGERATLSCRASQSVSSYLAWYQQKPGQAPR
LLIYDASNRATGIPARFSGSGSGTDFTLTISSLEPEDFAVYYCQQ
RSNWPPTFGQGTKVEIK.

In some embodiments, the anti-CD83 scFv comprises an amino acid sequence:

(SEQ ID NO: 170)
EVQLLESGGGLVQPGGSLRLSCAVSGFTFNSFAMSWVRQAPGKGLEWVSA
ISGSGGGTYYADSVKGRFTISRDNSKNTLYLQMNSLRAEDTAVYFCAKDK
ILWFGEPVFDYWGQGTLVTVSSGGGGSGGGGSGGGGSEIVLTQSPATLSL
SPGERATLSCRASQSVSSYLAWYQQKPGQAPRLLIYDASNRATGIPARFS
GSGSGTDFTLTISSLEPEDFAVYYCQQRSNWPPTFGQGTKVEIK.

### Anti-FLT3

In some embodiments, the anti-FLT3 scFv can comprise a variable heavy (V_{H}) domain having CDR1, CDR2 and CDR3 sequences and a variable light (V_{L}) domain having CDR1, CDR2 and CDR3 sequences.

Examples of anti-FLT3 scFvs polypeptides are disclosed in U.S. Pat. No. 10,961,312, which is incorporated by reference in its entirety for the teaching of these sequences.

For example, in some embodiments, the CDR1 sequence of the V_{H} domain comprises the amino acid sequence SYWMH (SEQ ID NO:171), CDR2 sequence of the V_{H} domain comprises the amino acid sequence EIDPSDSYKDYNQKFKD (SEQ ID NO:172), CDR3 sequence of the V_{H} domain comprises the amino acid sequence AITTTPFDF (SEQ ID NO:173), CDR1 sequence of the VL comprises the amino acid sequence RASQSISNNLH (SEQ ID NO:174), CDR2 sequence of the VL domain comprises the amino acid sequence YASQSIS (SEQ ID NO:175), and CDR3 sequence of the VL domain comprises the amino acid sequence QQSNTWPYT (SEQ ID NO:176).

In some embodiments, the anti-FLT3 scFv V_{H} domain has the amino acid sequence:

(SEQ ID NO: 177)
QVQLQQPGAELVKPGASLKLSCKSSGYTFTSYWMHWVRQRPGHGL
EWIGEIDPSDSYKDYNQKFKDKATLTVDRSSNTAYMHLSSLTSDD
SAVYYCARAITTTPFDFWGQGTTLTVSS,

and the anti-FLT3 scFv V_{L} domain has the amino acid sequence:

(SEQ ID NO: 178)
DIVLTQSPATLSVTPGDSVSLSCRASQSISNNLHWYQQKSHESPR
LLIKYASQSISGIPSRFSGSGSGTDFTLSINSVETEDFGVYFCQQ
SNTWPYTFGGGTKLEIKR.

In some embodiments, the anti-FLT3 scFv comprises an amino acid sequence:

(SEQ ID NO: 179)
QVQLQQPGAELVKPGASLKLSCKSSGYTFTSYWMHWVRQRPGHGLEWIGE
IDPSDSYKDYNQKFKDKATLTVDRSSNTAYMHLSSLTSDDSAVYYCARAI
TTTPFDFWGQGTTLTVSSGGGGSGGGGSGGGGSDIVLTQSPATLSVTPGD
SVSLSCRASQSISNNLHWYQQKSHESPRLLIKYASQSISGIPSRFSGSGS
GTDFTLSINSVETEDFGVYFCQQSNTWPYTFGGGTKLEIKR.

For example, in some embodiments, the CDR1 sequence of the V_{H} domain comprises the amino acid sequence NYGLH (SEQ ID NO:79), CDR2 sequence of the V_{H} domain comprises the amino acid sequence VIWSGGSTDYNAAFIS (SEQ ID NO:101), CDR3 sequence of the V_{H} domain comprises the amino acid sequence GGIYYANHYYAMDY (SEQ ID NO:114), CDR1 sequence of the VL comprises the amino acid sequence KSSQSLLNSGNQKNYM (SEQ ID NO:123), CDR2 sequence of the VL domain comprises the amino acid sequence GASTRES (SEQ ID NO:129), and CDR3 sequence of the VL domain comprises the amino acid sequence QNDHSYPLT (SEQ ID NO:139).

In some embodiments, the anti-FLT3 scFv V_{H} domain has the amino acid sequence:

(SEQ ID NO: 140)
QVQLKQSGPGLVQPSQSLSITCTVSGFSLTNYGLHWVRQSPGKGLEWLGV
IWSGGSTDYNAAFISRLSISKDNSKSQVFFKMNSLQADDTAIYYCARKGG
IYYANHYYAMDYWGQGTSVTVSS,

and the anti-FLT3 scFv V_{L} domain has the amino acid sequence:

(SEQ ID NO: 180)
DIVMTQSPSSLSVSAGEKVTMSCKSSQSLLNSGNQKNYMAWYQQKPGQPP
KLLIYGASTRESGVPDRFTGSGSGTDFTLTISSVQAEDLAVYYCQNDHSY
PLTFGAGTKLELKR.

In some embodiments, the anti-FLT3 scFv comprises an amino acid sequence:

(SEQ ID NO: 181)
QVQLKQSGPGLVQPSQSLSITCTVSGFSLTNYGLHWVRQSPGKGLEWLGV
IWSGGSTDYNAAFISRLSISKDNSKSQVFFKMNSLQADDTAIYYCARKGG
IYYANHYYAMDYWGQGTSVTVSSGGGGSGGGGSGGGGSDIVMTQSPSSLS
VSAGEKVTMSCKSSQSLLNSGNQKNYMAWYQQKPGQPPKLLIYGASTRES
GVPDRFTGSGSGTDFTLTISSVQAEDLAVYYCQNDHSYPLTFGAGTKLEL
KR.

Examples of anti-FLT3 scFvs and CAR polypeptides are disclosed in WO2018222935, which is incorporated by reference in its entirety for the teaching of these sequences.

### General Features

In some embodiments, the intracellular signaling domain is a CD3 zeta (CD3ζ) signaling domain. In some embodiments, the costimulatory signaling region comprises the cytoplasmic domain of CD28, 4-1BB, or a combination thereof. In some cases, the costimulatory signaling region contains 1, 2, 3, or 4 cytoplasmic domains of one or more intracellular signaling and/or costimulatory molecules. In some embodiments, the co-stimulatory signaling region contains one or more mutations in the cytoplasmic domains of CD28 and/or 4-1BB that enhance signaling.

In some embodiments, the CAR polypeptide contains an incomplete endodomain. For example, the CAR polypeptide can contain only an intracellular signaling domain or a co-stimulatory domain, but not both. In these embodiments, the immune effector cell is not activated unless it and a second CAR polypeptide (or endogenous T-cell receptor) that contains the missing domain both bind their respective antigens. Therefore, in some embodiments, the CAR polypeptide contains a CD3 zeta (CD3ζ) signaling domain but does not contain a costimulatory signaling region (CSR). In other embodiments, the CAR polypeptide contains the cytoplasmic domain of CD28, 4-1BB, or a combination thereof, but does not contain a CD3 zeta (CD3ζ) signaling domain (SD).

Additional CAR constructs are described, for example, in Fresnak A D, et al. Engineered T cells: the promise and challenges of cancer immunotherapy. Nat Rev Cancer. 2016 Aug. 23; 16(9):566-81, which is incorporated by reference in its entirety for the teaching of these CAR models.

For example, the CAR can be a TRUCK, Universal CAR, Self-driving CAR, Armored CAR, Self-destruct CAR, Conditional CAR, Marked CAR, TenCAR, Dual CAR, or sCAR.

CAR T cells engineered to be resistant to immunosuppression (Armored CARs) may be genetically modified to no longer express various immune checkpoint molecules (for example, cytotoxic T lymphocyte-associated antigen 4 (CTLA4) or programmed cell death protein 1 (PD1)), with an immune checkpoint switch receptor, or may be administered with a monoclonal antibody that blocks immune checkpoint signaling.

A self-destruct CAR may be designed using RNA delivered by electroporation to encode the CAR. Alternatively, inducible apoptosis of the T cell may be achieved based on ganciclovir binding to thymidine kinase in gene-modified lymphocytes or the more recently described system of activation of human caspase 9 by a small-molecule dimerizer.

A conditional CAR T cell is by default unresponsive, or switched ‘off’, until the addition of a small molecule to complete the circuit, enabling full transduction of both signal 1 and signal 2, thereby activating the CAR T cell. Alternatively, T cells may be engineered to express an adaptor-specific receptor with affinity for subsequently administered secondary antibodies directed at target antigen.

A tandem CAR (TanCAR) T cell expresses a single CAR consisting of two linked single-chain variable fragments (scFvs) that have different affinities fused to intracellular co-stimulatory domain(s) and a CD3ζ domain. TanCAR T cell activation is achieved only when target cells co-express both targets.

A dual CAR T cell expresses two separate CARs with different ligand binding targets; one CAR includes only the CD3ζ domain and the other CAR includes only the co-stimulatory domain(s). Dual CAR T cell activation requires co-expression of both targets.

A safety CAR (sCAR) consists of an extracellular scFv fused to an intracellular inhibitory domain. sCAR T cells co-expressing a standard CAR become activated only when encountering target cells that possess the standard CAR target but lack the sCAR target.

The antigen recognition domain of the disclosed CAR is usually an scFv. There are however many alternatives. An antigen recognition domain from native T-cell receptor (TCR) alpha and beta single chains have been described, as have simple ectodomains (e.g. CD4 ectodomain to recognize HIV infected cells) and more exotic recognition components such as a linked cytokine (which leads to recognition of cells bearing the cytokine receptor). In fact almost anything that binds a given target with high affinity can be used as an antigen recognition region.

The endodomain is the business end of the CAR that after antigen recognition transmits a signal to the immune effector cell, activating at least one of the normal effector functions of the immune effector cell. Effector function of a T cell, for example, may be cytolytic activity or helper activity including the secretion of cytokines. Therefore, the endodomain may comprise the “intracellular signaling domain” of a T cell receptor (TCR) and optional co-receptors. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal.

Cytoplasmic signaling sequences that regulate primary activation of the TCR complex that act in a stimulatory manner may contain signaling motifs which are known as immunoreceptor tyrosine-based activation motifs (ITAMs). Examples of ITAM containing cytoplasmic signaling sequences include those derived from CD8, CD3ζ, CD3δ, CD3γ, CD3ε, CD32 (Fc gamma RIIa), DAP10, DAP12, CD79a, CD79b, FcγRIγ, FcγRIIIγ, FcεRIβ (FCERIB), and FcεRIγ (FCERIG).

In particular embodiments, the intracellular signaling domain is derived from CD3 zeta (CD3ζ) (TCR zeta, GenBank acc no. BAG36664.1). T-cell surface glycoprotein CD3 zeta (CD3ζ) chain, also known as T-cell receptor T3 zeta chain or CD247 (Cluster of Differentiation 247), is a protein that in humans is encoded by the CD247 gene.

First-generation CARs typically had the intracellular domain from the CD3ζ chain, which is the primary transmitter of signals from endogenous TCRs. Second-generation CARs add intracellular signaling domains from various costimulatory protein receptors (e.g., CD28, 41BB, ICOS) to the endodomain of the CAR to provide additional signals to the T cell. More recent, third-generation CARs combine multiple signaling domains to further augment potency. T cells grafted with these CARs have demonstrated improved expansion, activation, persistence, and tumor-eradicating efficiency independent of costimulatory receptor/ligand interaction (Imai C, et al. Leukemia 2004 18:676-84; Maher J, et al. Nat Biotechnol 2002 20:70-5).

For example, the endodomain of the CAR can be designed to comprise the CD3ζ signaling domain by itself or combined with any other desired cytoplasmic domain(s) useful in the context of the CAR of the invention. For example, the cytoplasmic domain of the CAR can comprise a CD3ζ chain portion and a costimulatory signaling region. The costimulatory signaling region refers to a portion of the CAR comprising the intracellular domain of a costimulatory molecule. A costimulatory molecule is a cell surface molecule other than an antigen receptor or their ligands that is required for an efficient response of lymphocytes to an antigen. Examples of such molecules include CD27, CD28, 4-1BB (CD137), OX40, CD30, CD40, ICOS, lymphocyte function-associated antigen-1 (LFA-1), CD2, CD7, LIGHT, NKG2C, B7-H3, and a ligand that specifically binds with CD123, CD8, CD4, b2c, CD80, CD86, DAP10, DAP12, MyD88, BTNL3, and NKG2D. Thus, while the CAR is exemplified primarily with CD28 as the co-stimulatory signaling element, other costimulatory elements can be used alone or in combination with other co-stimulatory signaling elements.

In some embodiments, the CAR comprises a hinge sequence. A hinge sequence is a short sequence of amino acids that facilitates antibody flexibility (see, e.g., Woof et al., Nat. Rev. Immunol., 4(2): 89-99 (2004)). The hinge sequence may be positioned between the antigen recognition moiety (e.g., anti-CD83 scFv) and the transmembrane domain. The hinge sequence can be any suitable sequence derived or obtained from any suitable molecule. In some embodiments, for example, the hinge sequence is derived from a CD8a molecule or a CD28 molecule.

The transmembrane domain may be derived either from a natural or from a synthetic source. Where the source is natural, the domain may be derived from any membrane-bound or transmembrane protein. For example, the transmembrane region may be derived from (i.e. comprise at least the transmembrane region(s) of) the alpha, beta or zeta chain of the T-cell receptor, CD28, CD3 epsilon, CD45, CD4, CD5, CD8 (e.g., CD8 alpha, CD8 beta), CD9, CD16, CD22, CD33, CD37, CD64, CD80, CD86, CD134, CD137, or CD154, KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), CD160, CD19, IL2R beta, IL2R gamma, IL7R α, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, and PAG/Cbp. Alternatively the transmembrane domain may be synthetic, in which case it will comprise predominantly hydrophobic residues such as leucine and valine. In some cases, a triplet of phenylalanine, tryptophan and valine will be found at each end of a synthetic transmembrane domain. A short oligo-or polypeptide linker, such as between 2 and 10 amino acids in length, may form the linkage between the transmembrane domain and the endoplasmic domain of the CAR.

In some embodiments, the CAR has more than one transmembrane domain, which can be a repeat of the same transmembrane domain, or can be different transmembrane domains.

In some embodiments, the CAR is a multi-chain CAR, as described in WO2015/039523, which is incorporated by reference for this teaching. A multi-chain CAR can comprise separate extracellular ligand binding and signaling domains in different transmembrane polypeptides. The signaling domains can be designed to assemble in juxtamembrane position, which forms flexible architecture closer to natural receptors, that confers optimal signal transduction. For example, the multi-chain CAR can comprise a part of an FCERI alpha chain and a part of an FCERI beta chain such that the FCERI chains spontaneously dimerize together to form a CAR.

TABLE 1
Third Generation CARs lacking Signal Domain
(for dual CAR approach)

| | Co-stimulatory | Co-stimulatory | Signal |
|---|---|---|---|
| ScFv | Signal | Signal | Domain |
| CD83 or TA | CD28 | CD28 | none |
| CD83 or TA | CD28 | CD8 | none |
| CD83 or TA | CD28 | CD4 | none |
| CD83 or TA | CD28 | b2c | none |
| CD83 or TA | CD28 | CD137/41BB | none |
| CD83 or TA | CD28 | ICOS | none |
| CD83 or TA | CD28 | CD27 | none |
| CD83 or TA | CD28 | CD28δ | none |
| CD83 or TA | CD28 | CD80 | none |
| CD83 or TA | CD28 | CD86 | none |
| CD83 or TA | CD28 | OX40 | none |
| CD83 or TA | CD28 | DAP10 | none |
| CD83 or TA | CD28 | MyD88 | none |
| CD83 or TA | CD28 | CD7 | none |
| CD83 or TA | CD28 | DAP12 | none |
| CD83 or TA | CD28 | MyD88 | none |
| CD83 or TA | CD28 | CD7 | none |
| CD83 or TA | CD8 | CD28 | none |
| CD83 or TA | CD8 | CD8 | none |
| CD83 or TA | CD8 | CD4 | none |
| CD83 or TA | CD8 | b2c | none |
| CD83 or TA | CD8 | CD137/41BB | none |
| CD83 or TA | CD8 | ICOS | none |
| CD83 or TA | CD8 | CD27 | none |
| CD83 or TA | CD8 | CD28δ | none |
| CD83 or TA | CD8 | CD80 | none |
| CD83 or TA | CD8 | CD86 | none |
| CD83 or TA | CD8 | OX40 | none |
| CD83 or TA | CD8 | DAP10 | none |
| CD83 or TA | CD8 | MyD88 | none |
| CD83 or TA | CD8 | CD7 | none |
| CD83 or TA | CD8 | DAP12 | none |
| CD83 or TA | CD8 | MyD88 | none |
| CD83 or TA | CD8 | CD7 | none |
| CD83 or TA | CD4 | CD28 | none |
| CD83 or TA | CD4 | CD8 | none |
| CD83 or TA | CD4 | CD4 | none |
| CD83 or TA | CD4 | b2c | none |
| CD83 or TA | CD4 | CD137/41BB | none |
| CD83 or TA | CD4 | ICOS | none |
| CD83 or TA | CD4 | CD27 | none |
| CD83 or TA | CD4 | CD28δ | none |
| CD83 or TA | CD4 | CD80 | none |
| CD83 or TA | CD4 | CD86 | none |
| CD83 or TA | CD4 | OX40 | none |
| CD83 or TA | CD4 | DAP10 | none |
| CD83 or TA | CD4 | MyD88 | none |
| CD83 or TA | CD4 | CD7 | none |
| CD83 or TA | CD4 | DAP12 | none |
| CD83 or TA | CD4 | MyD88 | none |
| CD83 or TA | CD4 | CD7 | none |
| CD83 or TA | b2c | CD28 | none |
| CD83 or TA | b2c | CD8 | none |
| CD83 or TA | b2c | CD4 | none |
| CD83 or TA | b2c | b2c | none |
| CD83 or TA | b2c | CD137/41BB | none |
| CD83 or TA | b2c | ICOS | none |
| CD83 or TA | b2c | CD27 | none |
| CD83 or TA | b2c | CD28δ | none |
| CD83 or TA | b2c | CD80 | none |
| CD83 or TA | b2c | CD86 | none |
| CD83 or TA | b2c | OX40 | none |
| CD83 or TA | b2c | DAP10 | none |
| CD83 or TA | b2c | MyD88 | none |
| CD83 or TA | b2c | CD7 | none |
| CD83 or TA | b2c | DAP12 | none |
| CD83 or TA | b2c | MyD88 | none |
| CD83 or TA | b2c | CD7 | none |
| CD83 or TA | CD137/41BB | CD28 | none |
| CD83 or TA | CD137/41BB | CD8 | none |
| CD83 or TA | CD137/41BB | CD4 | none |
| CD83 or TA | CD137/41BB | b2c | none |
| CD83 or TA | CD137/41BB | CD137/41BB | none |
| CD83 or TA | CD137/41BB | ICOS | none |
| CD83 or TA | CD137/41BB | CD27 | none |
| CD83 or TA | CD137/41BB | CD28δ | none |
| CD83 or TA | CD137/41BB | CD80 | none |
| CD83 or TA | CD137/41BB | CD86 | none |
| CD83 or TA | CD137/41BB | OX40 | none |
| CD83 or TA | CD137/41BB | DAP10 | none |
| CD83 or TA | CD137/41BB | MyD88 | none |
| CD83 or TA | CD137/41BB | CD7 | none |
| CD83 or TA | CD137/41BB | DAP12 | none |
| CD83 or TA | CD137/41BB | MyD88 | none |
| CD83 or TA | CD137/41BB | CD7 | none |
| CD83 or TA | ICOS | CD28 | none |
| CD83 or TA | ICOS | CD8 | none |
| CD83 or TA | ICOS | CD4 | none |
| CD83 or TA | ICOS | b2c | none |
| CD83 or TA | ICOS | CD137/41BB | none |
| CD83 or TA | ICOS | ICOS | none |
| CD83 or TA | ICOS | CD27 | none |
| CD83 or TA | ICOS | CD28δ | none |
| CD83 or TA | ICOS | CD80 | none |
| CD83 or TA | ICOS | CD86 | none |
| CD83 or TA | ICOS | OX40 | none |
| CD83 or TA | ICOS | DAP10 | none |
| CD83 or TA | ICOS | MyD88 | none |
| CD83 or TA | ICOS | CD7 | none |
| CD83 or TA | ICOS | DAP12 | none |
| CD83 or TA | ICOS | MyD88 | none |
| CD83 or TA | ICOS | CD7 | none |
| CD83 or TA | ICOS | CD28 | none |
| CD83 or TA | ICOS | CD8 | none |
| CD83 or TA | ICOS | CD4 | none |
| CD83 or TA | ICOS | b2c | none |
| CD83 or TA | ICOS | CD137/41BB | none |
| CD83 or TA | ICOS | ICOS | none |
| CD83 or TA | ICOS | CD27 | none |
| CD83 or TA | ICOS | CD28δ | none |
| CD83 or TA | ICOS | CD80 | none |
| CD83 or TA | ICOS | CD86 | none |
| CD83 or TA | ICOS | OX40 | none |
| CD83 or TA | ICOS | DAP10 | none |
| CD83 or TA | ICOS | MyD88 | none |
| CD83 or TA | ICOS | CD7 | none |
| CD83 or TA | ICOS | DAP12 | none |
| CD83 or TA | ICOS | MyD88 | none |
| CD83 or TA | ICOS | CD7 | none |
| CD83 or TA | CD27 | CD28 | none |
| CD83 or TA | CD27 | CD8 | none |
| CD83 or TA | CD27 | CD4 | none |
| CD83 or TA | CD27 | b2c | none |
| CD83 or TA | CD27 | CD137/41BB | none |
| CD83 or TA | CD27 | ICOS | none |
| CD83 or TA | CD27 | CD27 | none |
| CD83 or TA | CD27 | CD28δ | none |
| CD83 or TA | CD27 | CD80 | none |
| CD83 or TA | CD27 | CD86 | none |
| CD83 or TA | CD27 | OX40 | none |
| CD83 or TA | CD27 | DAP10 | none |
| CD83 or TA | CD27 | MyD88 | none |
| CD83 or TA | CD27 | CD7 | none |
| CD83 or TA | CD27 | DAP12 | none |
| CD83 or TA | CD27 | MyD88 | none |
| CD83 or TA | CD27 | CD7 | none |
| CD83 or TA | CD28δ | CD28 | none |
| CD83 or TA | CD28δ | CD8 | none |
| CD83 or TA | CD28δ | CD4 | none |
| CD83 or TA | CD28δ | b2c | none |
| CD83 or TA | CD28δ | CD137/41BB | none |
| CD83 or TA | CD28δ | ICOS | none |
| CD83 or TA | CD28δ | CD27 | none |
| CD83 or TA | CD28δ | CD28δ | none |
| CD83 or TA | CD28δ | CD80 | none |
| CD83 or TA | CD28δ | CD86 | none |
| CD83 or TA | CD28δ | OX40 | none |
| CD83 or TA | CD28δ | DAP10 | none |
| CD83 or TA | CD28δ | MyD88 | none |
| CD83 or TA | CD28δ | CD7 | none |
| CD83 or TA | CD28δ | DAP12 | none |
| CD83 or TA | CD28δ | MyD88 | none |
| CD83 or TA | CD28δ | CD7 | none |
| CD83 or TA | CD80 | CD28 | none |
| CD83 or TA | CD80 | CD8 | none |
| CD83 or TA | CD80 | CD4 | none |
| CD83 or TA | CD80 | b2c | none |
| CD83 or TA | CD80 | CD137/41BB | none |
| CD83 or TA | CD80 | ICOS | none |
| CD83 or TA | CD80 | CD27 | none |
| CD83 or TA | CD80 | CD28δ | none |
| CD83 or TA | CD80 | CD80 | none |
| CD83 or TA | CD80 | CD86 | none |
| CD83 or TA | CD80 | OX40 | none |
| CD83 or TA | CD80 | DAP10 | none |
| CD83 or TA | CD80 | MyD88 | none |
| CD83 or TA | CD80 | CD7 | none |
| CD83 or TA | CD80 | DAP12 | none |
| CD83 or TA | CD80 | MyD88 | none |
| CD83 or TA | CD80 | CD7 | none |
| CD83 or TA | CD86 | CD28 | none |
| CD83 or TA | CD86 | CD8 | none |
| CD83 or TA | CD86 | CD4 | none |
| CD83 or TA | CD86 | b2c | none |
| CD83 or TA | CD86 | CD137/41BB | none |
| CD83 or TA | CD86 | ICOS | none |
| CD83 or TA | CD86 | CD27 | none |
| CD83 or TA | CD86 | CD28δ | none |
| CD83 or TA | CD86 | CD80 | none |
| CD83 or TA | CD86 | CD86 | none |
| CD83 or TA | CD86 | OX40 | none |
| CD83 or TA | CD86 | DAP10 | none |
| CD83 or TA | CD86 | MyD88 | none |
| CD83 or TA | CD86 | CD7 | none |
| CD83 or TA | CD86 | DAP12 | none |
| CD83 or TA | CD86 | MyD88 | none |
| CD83 or TA | CD86 | CD7 | none |
| CD83 or TA | OX40 | CD28 | none |
| CD83 or TA | OX40 | CD8 | none |
| CD83 or TA | OX40 | CD4 | none |
| CD83 or TA | OX40 | b2c | none |
| CD83 or TA | OX40 | CD137/41BB | none |
| CD83 or TA | OX40 | ICOS | none |
| CD83 or TA | OX40 | CD27 | none |
| CD83 or TA | OX40 | CD28δ | none |
| CD83 or TA | OX40 | CD80 | none |
| CD83 or TA | OX40 | CD86 | none |
| CD83 or TA | OX40 | OX40 | none |
| CD83 or TA | OX40 | DAP10 | none |
| CD83 or TA | OX40 | MyD88 | none |
| CD83 or TA | OX40 | CD7 | none |
| CD83 or TA | OX40 | DAP12 | none |
| CD83 or TA | OX40 | MyD88 | none |
| CD83 or TA | OX40 | CD7 | none |
| CD83 or TA | DAP10 | CD28 | none |
| CD83 or TA | DAP10 | CD8 | none |
| CD83 or TA | DAP10 | CD4 | none |
| CD83 or TA | DAP10 | b2c | none |
| CD83 or TA | DAP10 | CD137/41BB | none |
| CD83 or TA | DAP10 | ICOS | none |
| CD83 or TA | DAP10 | CD27 | none |
| CD83 or TA | DAP10 | CD28δ | none |
| CD83 or TA | DAP10 | CD80 | none |
| CD83 or TA | DAP10 | CD86 | none |
| CD83 or TA | DAP10 | OX40 | none |
| CD83 or TA | DAP10 | DAP10 | none |
| CD83 or TA | DAP10 | MyD88 | none |
| CD83 or TA | DAP10 | CD7 | none |
| CD83 or TA | DAP10 | DAP12 | none |
| CD83 or TA | DAP10 | MyD88 | none |
| CD83 or TA | DAP10 | CD7 | none |
| CD83 or TA | DAP12 | CD28 | none |
| CD83 or TA | DAP12 | CD8 | none |
| CD83 or TA | DAP12 | CD4 | none |
| CD83 or TA | DAP12 | b2c | none |
| CD83 or TA | DAP12 | CD137/41BB | none |
| CD83 or TA | DAP12 | ICOS | none |
| CD83 or TA | DAP12 | CD27 | none |
| CD83 or TA | DAP12 | CD28δ | none |
| CD83 or TA | DAP12 | CD80 | none |
| CD83 or TA | DAP12 | CD86 | none |
| CD83 or TA | DAP12 | OX40 | none |
| CD83 or TA | DAP12 | DAP10 | none |
| CD83 or TA | DAP12 | MyD88 | none |
| CD83 or TA | DAP12 | CD7 | none |
| CD83 or TA | DAP12 | DAP12 | none |
| CD83 or TA | DAP12 | MyD88 | none |
| CD83 or TA | DAP12 | CD7 | none |
| CD83 or TA | MyD88 | CD28 | none |
| CD83 or TA | MyD88 | CD8 | none |
| CD83 or TA | MyD88 | CD4 | none |
| CD83 or TA | MyD88 | b2c | none |
| CD83 or TA | MyD88 | CD137/41BB | none |
| CD83 or TA | MyD88 | ICOS | none |
| CD83 or TA | MyD88 | CD27 | none |
| CD83 or TA | MyD88 | CD28δ | none |
| CD83 or TA | MyD88 | CD80 | none |
| CD83 or TA | MyD88 | CD86 | none |
| CD83 or TA | MyD88 | OX40 | none |
| CD83 or TA | MyD88 | DAP10 | none |
| CD83 or TA | MyD88 | MyD88 | none |
| CD83 or TA | MyD88 | CD7 | none |
| CD83 or TA | MyD88 | DAP12 | none |
| CD83 or TA | MyD88 | MyD88 | none |
| CD83 or TA | MyD88 | CD7 | none |
| CD83 or TA | CD7 | CD28 | none |
| CD83 or TA | CD7 | CD8 | none |
| CD83 or TA | CD7 | CD4 | none |
| CD83 or TA | CD7 | b2c | none |
| CD83 or TA | CD7 | CD137/41BB | none |
| CD83 or TA | CD7 | ICOS | none |
| CD83 or TA | CD7 | CD27 | none |
| CD83 or TA | CD7 | CD28δ | none |
| CD83 or TA | CD7 | CD80 | none |
| CD83 or TA | CD7 | CD86 | none |
| CD83 or TA | CD7 | OX40 | none |
| CD83 or TA | CD7 | DAP10 | none |
| CD83 or TA | CD7 | MyD88 | none |
| CD83 or TA | CD7 | CD7 | none |
| CD83 or TA | CD7 | DAP12 | none |
| CD83 or TA | CD7 | MyD88 | none |
| CD83 or TA | CD7 | CD7 | none |
| CD83 or TA | BTNL3 | CD28 | none |
| CD83 or TA | BTNL3 | CD8 | none |
| CD83 or TA | BTNL3 | CD4 | none |
| CD83 or TA | BTNL3 | b2c | none |
| CD83 or TA | BTNL3 | CD137/41BB | none |
| CD83 or TA | BTNL3 | ICOS | none |
| CD83 or TA | BTNL3 | CD27 | none |
| CD83 or TA | BTNL3 | CD28δ | none |
| CD83 or TA | BTNL3 | CD80 | none |
| CD83 or TA | BTNL3 | CD86 | none |
| CD83 or TA | BTNL3 | OX40 | none |
| CD83 or TA | BTNL3 | DAP10 | none |
| CD83 or TA | BTNL3 | MyD88 | none |
| CD83 or TA | BTNL3 | CD7 | none |
| CD83 or TA | BTNL3 | DAP12 | none |
| CD83 or TA | BTNL3 | MyD88 | none |
| CD83 or TA | BTNL3 | CD7 | none |
| CD83 or TA | NKG2D | CD28 | none |
| CD83 or TA | NKG2D | CD8 | none |
| CD83 or TA | NKG2D | CD4 | none |
| CD83 or TA | NKG2D | b2c | none |
| CD83 or TA | NKG2D | CD137/41BB | none |
| CD83 or TA | NKG2D | ICOS | none |
| CD83 or TA | NKG2D | CD27 | none |
| CD83 or TA | NKG2D | CD28δ | none |
| CD83 or TA | NKG2D | CD80 | none |
| CD83 or TA | NKG2D | CD86 | none |
| CD83 or TA | NKG2D | OX40 | none |
| CD83 or TA | NKG2D | DAP10 | none |
| CD83 or TA | NKG2D | MyD88 | none |
| CD83 or TA | NKG2D | CD7 | none |
| CD83 or TA | NKG2D | DAP12 | none |
| CD83 or TA | NKG2D | MyD88 | none |
| CD83 or TA | NKG2D | CD7 | none |

In some embodiments, the anti-CD83 and/or anti-TA binding agent is single chain variable fragment (scFv) antibody. The affinity/specificity of an scFv is driven in large part by specific sequences within complementarity determining regions (CDRs) in the heavy (V_{H}) and light (V_{L}) chain. Each V_{H} and V_{L} sequence will have three CDRs (CDR1, CDR2, CDR3).

In some embodiments, the binding agent is derived from natural antibodies, such as monoclonal antibodies. In some cases, the antibody is human. In some cases, the antibody has undergone an alteration to render it less immunogenic when administered to humans. For example, the alteration comprises one or more techniques selected from the group consisting of chimerization, humanization, CDR-grafting, deimmunization, and mutation of framework amino acids to correspond to the closest human germline sequence.

### Nucleic Acids and Vectors

Also disclosed are polynucleotides and polynucleotide vectors encoding the disclosed CARs. Nucleic acid sequences encoding the disclosed CARs, and regions thereof, can be obtained using recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than cloned.

Expression of nucleic acids encoding CARs is typically achieved by operably linking a nucleic acid encoding the CAR polypeptide to a promoter, and incorporating the construct into an expression vector. Typical cloning vectors contain transcription and translation terminators, initiation sequences, and promoters useful for regulation of the expression of the desired nucleic acid sequence.

The disclosed nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, for example, in Sambrook et al. (2001, Molecular Cloning: A Laboratory Manual, Cold Spring Harbor Laboratory, New York), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers. In some embodiments, the polynucleotide vectors are lentiviral or retroviral vectors.

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1α (EF-1α). However, other constitutive promoter sequences may also be used, including, but not limited to the simian virus 40 (SV40) early promoter, MND (myeloproliferative sarcoma virus) promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. The promoter can alternatively be an inducible promoter. Examples of inducible promoters include, but are not limited to a metallothionine promoter, a glucocorticoid promoter, a progesterone promoter, and a tetracycline promoter.

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another.

In order to assess the expression of a CAR polypeptide or portions thereof, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene. Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001, Molecular Cloning: A Laboratory Manual, Cold Spring Harbor Laboratory, New York).

Biological methods for introducing a polynucleotide of interest into a host cell include the use of DNA and RNA vectors. Viral vectors, and especially retroviral vectors, have become the most widely used method for inserting genes into mammalian, e.g., human cells.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes. Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc, (Birmingham, Ala.).

### Immune Effector Cells

Also disclosed are immune effector cells that are engineered to express the disclosed CARs (also referred to herein as “CAR-T cells.” These cells are preferably obtained from the subject to be treated (i.e. are autologous). However, in some embodiments, immune effector cell lines or donor effector cells (allogeneic) are used. Immune effector cells can be obtained from a number of sources, including peripheral blood mononuclear cells, bone marrow, lymph node tissue, cord blood, thymus tissue, tissue from a site of infection, ascites, pleural effusion, spleen tissue, and tumors. Immune effector cells can be obtained from blood collected from a subject using any number of techniques known to the skilled artisan, such as Ficoll™ separation. For example, cells from the circulating blood of an individual may be obtained by apheresis. In some embodiments, immune effector cells are isolated from peripheral blood lymphocytes by lysing the red blood cells and depleting the monocytes, for example, by centrifugation through a PERCOLL™ gradient or by counterflow centrifugal elutriation. A specific subpopulation of immune effector cells can be further isolated by positive or negative selection techniques. For example, immune effector cells can be isolated using a combination of antibodies directed to surface markers unique to the positively selected cells, e.g., by incubation with antibody-conjugated beads for a time period sufficient for positive selection of the desired immune effector cells. Alternatively, enrichment of immune effector cells population can be accomplished by negative selection using a combination of antibodies directed to surface markers unique to the negatively selected cells.

In some embodiments, the immune effector cells comprise any leukocyte involved in defending the body against infectious disease and foreign materials. For example, the immune effector cells can comprise lymphocytes, monocytes, macrophages, dentritic cells, mast cells, neutrophils, basophils, eosinophils, or any combinations thereof. For example, the immune effector cells can comprise T lymphocytes.

T cells or T lymphocytes can be distinguished from other lymphocytes, such as B cells and natural killer cells (NK cells), by the presence of a T-cell receptor (TCR) on the cell surface. They are called T cells because they mature in the thymus (although some also mature in the tonsils). There are several subsets of T cells, each with a distinct function.

T helper cells (T_{H} cells) assist other white blood cells in immunologic processes, including maturation of B cells into plasma cells and memory B cells, and activation of cytotoxic T cells and macrophages. These cells are also known as CD4+ T cells because they express the CD4 glycoprotein on their surface. Helper T cells become activated when they are presented with peptide antigens by MHC class II molecules, which are expressed on the surface of antigen-presenting cells (APCs). Once activated, they divide rapidly and secrete small proteins called cytokines that regulate or assist in the active immune response. These cells can differentiate into one of several subtypes, including T_{H}1, T_{H}2, T_{H}3, T_{H}17, T_{H}9, or T_{FH}, which secrete different cytokines to facilitate a different type of immune response.

Cytotoxic T cells (T_{c} cells, or CTLs) destroy virally infected cells and tumor cells, and are also implicated in transplant rejection. These cells are also known as CD8⁺ T cells since they express the CD8 glycoprotein at their surface. These cells recognize their targets by binding to antigen associated with MHC class I molecules, which are present on the surface of all nucleated cells. Through IL-10, adenosine and other molecules secreted by regulatory T cells, the CD8+ cells can be inactivated to an anergic state, which prevents autoimmune diseases.

Memory T cells are a subset of antigen-specific T cells that persist long-term after an infection has resolved. They quickly expand to large numbers of effector T cells upon re-exposure to their cognate antigen, thus providing the immune system with “memory” against past infections. Memory cells may be either CD4⁺ or CD8⁺. Memory T cells typically express the cell surface protein CD45RO.

Regulatory T cells (T_{reg} cells), formerly known as suppressor T cells, are crucial for the maintenance of immunological tolerance. Their major role is to shut down T cell-mediated immunity toward the end of an immune reaction and to suppress auto-reactive T cells that escaped the process of negative selection in the thymus. Two major classes of CD4⁺ T_{reg} cells have been described—naturally occurring T_{reg} cells and adaptive T_{reg} cells.

Natural killer T (NKT) cells (not to be confused with natural killer (NK) cells) bridge the adaptive immune system with the innate immune system. Unlike conventional T cells that recognize peptide antigens presented by major histocompatibility complex (MHC) molecules, NKT cells recognize glycolipid antigen presented by a molecule called CD1d.

In some embodiments, the T cells comprise a mixture of CD4+ cells. In other embodiments, the T cells are enriched for one or more subsets based on cell surface expression. For example, in some cases, the T comprise are cytotoxic CD8⁺ T lymphocytes. In some embodiments, the T cells comprise γδ T cells, which possess a distinct T-cell receptor (TCR) having one γ chain and one δ chain instead of α and β chains.

Natural-killer (NK) cells are CD56⁺CD3⁻ large granular lymphocytes that can kill virally infected and transformed cells, and constitute a critical cellular subset of the innate immune system (Godfrey J, et al. Leuk Lymphoma 2012 53:1666-1676). Unlike cytotoxic CD8⁺ T lymphocytes, NK cells launch cytotoxicity against tumor cells without the requirement for prior sensitization, and can also eradicate MHC-I-negative cells (Narni-Mancinelli E, et al. Int Immunol 2011 23:427-431). NK cells are safer effector cells, as they may avoid the potentially lethal complications of cytokine storms (Morgan R A, et al. Mol Ther 2010 18:843-851), tumor lysis syndrome (Porter D L, et al. N Engl J Med 2011 365:725-733), and on-target, off-tumor effects.

### Therapeutic Methods

The disclosed CAR-modified immune effector cells may be administered either alone, or as a pharmaceutical composition in combination with diluents and/or with other components such as IL-2, IL-15, or other cytokines or cell populations.

In some embodiments, the disclosed CAR-modified immune effector cells are administered in combination with ER stress blockade (compounds to target the IRE-1/XBP-1 pathway (e.g., B-I09). In some embodiments, the disclosed CAR-modified immune effector cells are administered in combination with a JAK2 inhibitor, a STAT3 inhibitor, an Aurora kinase inhibitor, an mTOR inhibitor, or any combination thereof.

Briefly, pharmaceutical compositions may comprise a target cell population as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions for use in the disclosed methods are in some embodiments formulated for intravenous administration. Pharmaceutical compositions may be administered in any manner appropriate treat MM. The quantity and frequency of administration will be determined by such factors as the condition of the patient, and the severity of the patient's disease, although appropriate dosages may be determined by clinical trials.

When a “therapeutic amount” is indicated, the precise amount of the compositions of the present invention to be administered can be determined by a physician with consideration of individual differences in age, weight, extent of transplantation, and condition of the patient (subject). It can generally be stated that a pharmaceutical composition comprising the T cells described herein may be administered at a dosage of 10⁴ to 10⁹ cells/kg body weight, such as 10⁵ to 10⁶ cells/kg body weight, including all integer values within those ranges. T cell compositions may also be administered multiple times at these dosages. The cells can be administered by using infusion techniques that are commonly known in immunotherapy (see, e.g., Rosenberg et al., New Eng. J. of Med. 319:1676, 1988). The optimal dosage and treatment regime for a particular patient can readily be determined by one skilled in the art of medicine by monitoring the patient for signs of disease and adjusting the treatment accordingly.

In certain embodiments, it may be desired to administer activated T cells to a subject and then subsequently re-draw blood (or have an apheresis performed), activate T cells therefrom according to the disclosed methods, and reinfuse the patient with these activated and expanded T cells. This process can be carried out multiple times every few weeks. In certain embodiments, T cells can be activated from blood draws of from 10 cc to 400 cc. In certain embodiments, T cells are activated from blood draws of 20 cc, 30 cc, 40 cc, 50 cc, 60 cc, 70 cc, 80 cc, 90 cc, or 100 cc. Using this multiple blood draw/multiple reinfusion protocol may serve to select out certain populations of T cells.

The administration of the disclosed compositions may be carried out in any convenient manner, including by injection, transfusion, or implantation. The compositions described herein may be administered to a patient subcutaneously, intradermally, intranodally, intramedullary, intramuscularly, by intravenous (i.v.) injection, or intraperitoneally. In some embodiments, the disclosed compositions are administered to a patient by intradermal or subcutaneous injection. In some embodiments, the disclosed compositions are administered by i.v. injection. The compositions may also be injected directly into a site of transplantation.

In certain embodiments, the disclosed CAR-modified immune effector cells are administered to a patient in conjunction with (e.g., before, simultaneously or following) any number of relevant treatment modalities, including but not limited to thalidomide, dexamethasone, bortezomib, and lenalidomide. In further embodiments, the CAR-modified immune effector cells may be used in combination with chemotherapy, radiation, immunosuppressive agents, such as cyclosporin, azathioprine, methotrexate, mycophenolate, and FK506, antibodies, or other immunoablative agents such as CAM PATH, anti-CD3 antibodies or other antibody therapies, cytoxin, fludaribine, cyclosporin, FK506, rapamycin, mycophenolic acid, steroids, FR901228, cytokines, and irradiation. In some embodiments, the CAR-modified immune effector cells are administered to a patient in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH. In another embodiment, the cell compositions of the present invention are administered following B-cell ablative therapy such as agents that react with CD20, e.g., Rituxan. For example, in some embodiments, subjects may undergo standard treatment with high dose chemotherapy followed by peripheral blood stem cell transplantation. In certain embodiments, following the transplant, subjects receive an infusion of the expanded immune cells of the present invention. In an additional embodiment, expanded cells are administered before or following surgery.

One primary concern with CAR-T cells as a form of “living therapeutic” is their manipulability in vivo and their potential immune-stimulating side effects. To better control CAR-T therapy and prevent against unwanted side effects, a variety of features have been engineered including off-switches, safety mechanisms, and conditional control mechanisms. Both self-destruct and marked/tagged CAR-T cells for example, are engineered to have an “off-switch” that promotes clearance of the CAR-expressing T-cell. A self-destruct CAR-T contains a CAR, but is also engineered to express a pro-apoptotic suicide gene or “elimination gene” inducible upon administration of an exogenous molecule. A variety of suicide genes may be employed for this purpose, including HSV-TK (herpes simplex virus thymidine kinase), Fas, iCasp9 (inducible caspase 9), CD20, MYC TAG, and truncated EGFR (endothelial growth factor receptor). HSK for example, will convert the prodrug ganciclovir (GCV) into GCV-triphosphate that incorporates itself into replicating DNA, ultimately leading to cell death. iCasp9 is a chimeric protein containing components of FK506-binding protein that binds the small molecule AP1903, leading to caspase 9 dimerization and apoptosis. A marked/tagged CAR-T cell however, is one that possesses a CAR but also is engineered to express a selection marker. Administration of a mAb against this selection marker will promote clearance of the CAR-T cell. Truncated EGFR is one such targetable antigen by the anti-EGFR mAb, and administration of cetuximab works to promotes elimination of the CAR-T cell. CARs created to have these features are also referred to as sCARs for ‘switchable CARs’, and RCARs for ‘regulatable CARs’. A “safety CAR”, also known as an “inhibitory CAR” (iCAR), is engineered to express two antigen binding domains. One of these extracellular domains is directed against a first antigen and bound to an intracellular costimulatory and stimulatory domain. The second extracellular antigen binding domain however is specific for normal tissue and bound to an intracellular checkpoint domain such as CTLA4, PD1, or CD45. Incorporation of multiple intracellular inhibitory domains to the iCAR is also possible. Some inhibitory molecules that may provide these inhibitory domains include B7-H1, B7-1, CD160, PIH, 2B4, CEACAM (CEACAM-1. CEACAM-3, and/or CEACAM-5), LAG-3, TIGIT, BTLA, LAIR1, and TGFβ-R. In the presence of normal tissue, stimulation of this second antigen binding domain will work to inhibit the CAR. It should be noted that due to this dual antigen specificity, iCARs are also a form of bi-specific CAR-T cells. The safety CAR-T engineering enhances specificity of the CAR-T cell for tissue, and is advantageous in situations where certain normal tissues may express very low levels of a antigen that would lead to off target effects with a standard CAR (Morgan 2010). A conditional CAR-T cell expresses an extracellular antigen binding domain connected to an intracellular costimulatory domain and a separate, intracellular costimulator. The costimulatory and stimulatory domain sequences are engineered in such a way that upon administration of an exogenous molecule the resultant proteins will come together intracellularly to complete the CAR circuit. In this way, CAR-T activation can be modulated, and possibly even ‘fine-tuned’ or personalized to a specific patient. Similar to a dual CAR design, the stimulatory and costimulatory domains are physically separated when inactive in the conditional CAR; for this reason these too are also referred to as a “split CAR”.

Typically, CAR-T cells are created using α-β T cells, however γ-δ T cells may also be used. In some embodiments, the described CAR constructs, domains, and engineered features used to generate CAR-T cells could similarly be employed in the generation of other types of CAR-expressing immune cells including NK (natural killer) cells, B cells, mast cells, myeloid-derived phagocytes, and NKT cells. Alternatively, a CAR-expressing cell may be created to have properties of both T-cell and NK cells. In an additional embodiment, the transduced with CARs may be autologous or allogeneic.

Several different methods for CAR expression may be used including retroviral transduction (including γ-retroviral), lentiviral transduction, transposon/transposases (Sleeping Beauty and PiggyBac systems), and messenger RNA transfer-mediated gene expression. Gene editing (gene insertion or gene deletion/disruption) has become of increasing importance with respect to the possibility for engineering CAR-T cells as well. CRISPR-Cas9, ZFN (zinc finger nuclease), and TALEN (transcription activator like effector nuclease) systems are three potential methods through which CAR-T cells may be generated.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1: B Cell Acute Lymphoblastic Leukemia

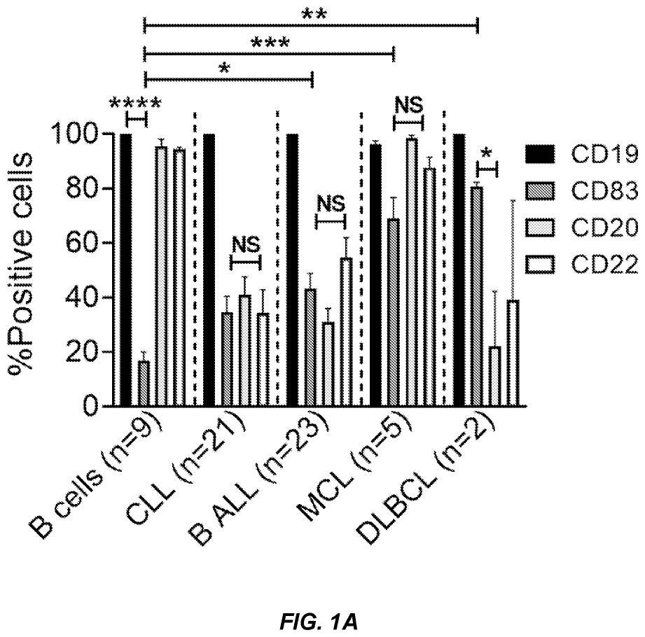

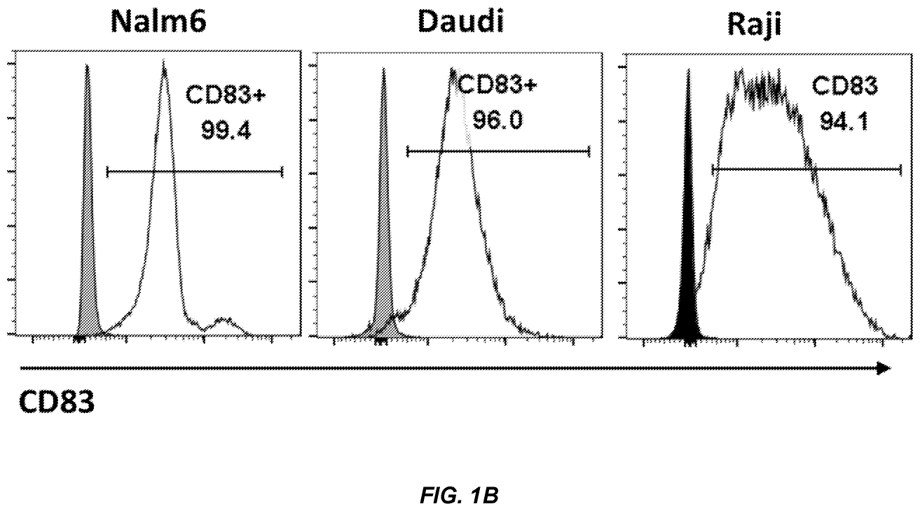

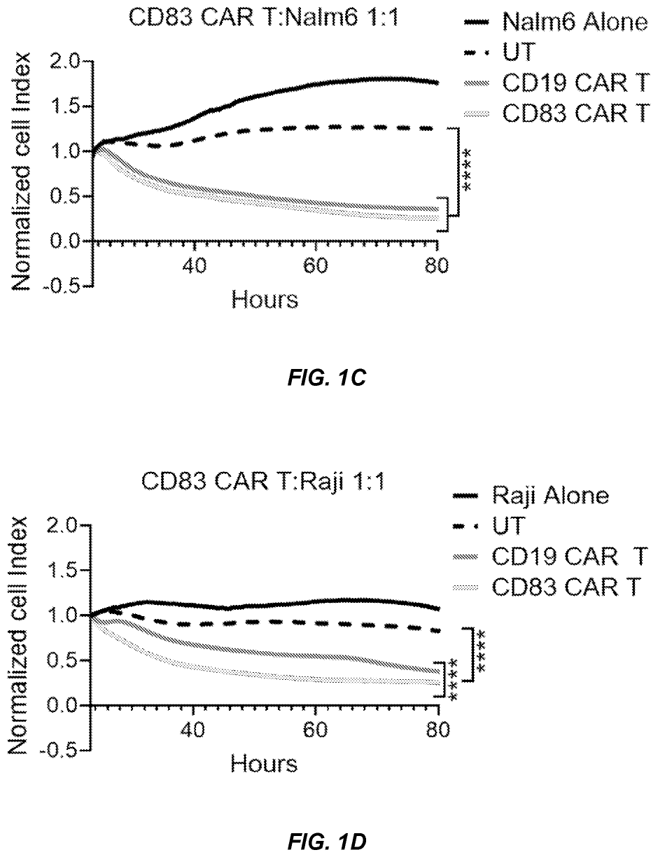

CD83 is present on Reed-Sternberg cells in Hodgkin lymphoma (Li Z, et al. Haematologica 2018 103(4):655-65). This led to investigation of whether B cell malignancies may express CD83, rendering B cell ALL sensitive to CD83 CAR T therapy. Preliminary data provides evidence that CD83 is differentially expressed on multiple B cell malignancies, including ALL, compared to exceptionally low expression on circulating healthy B cells (FIG. 1A). CD83 is also robustly expressed on Nalm6, Raji, and Daudi B lymphoblast cell lines (FIG. 1B). Further, CD83 CAR T significantly kill Nalm6 and Raji cells with potency at least as effective as CD19 CAR T (FIG. 1C,1D), highlighting the relevance of CD83 in targeting B cell malignancies. Prior exposure to CD19 CAR T can lead to CD19 antigen loss overtime or during B cell ALL relapse. However, CD19 CAR T can be a useful bridge to alloHCT for curative intent. Notably, relapsed ALL after alloHCT carries a dismal prognosis, and concurrent CD19 antigen loss creates a dire lack of therapeutic options in heavily pretreated individuals. Identifying novel CAR targets for B cell ALL, such as CD83, is a critical unmet need for cancer patients. It was hypothesize that CD83-directed cell therapy will prevent ALL relapse and GVHD after alloHCT.

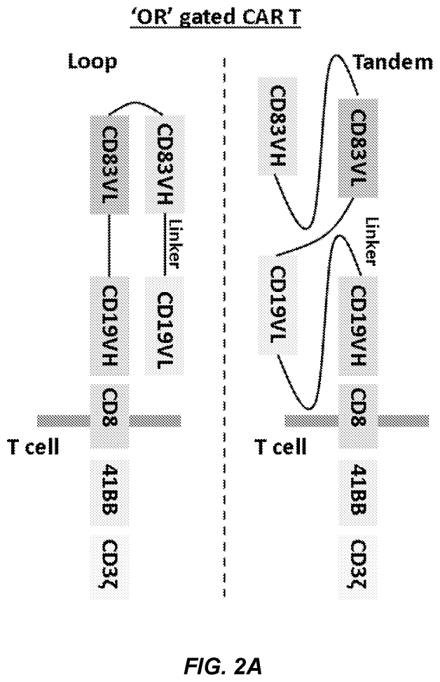

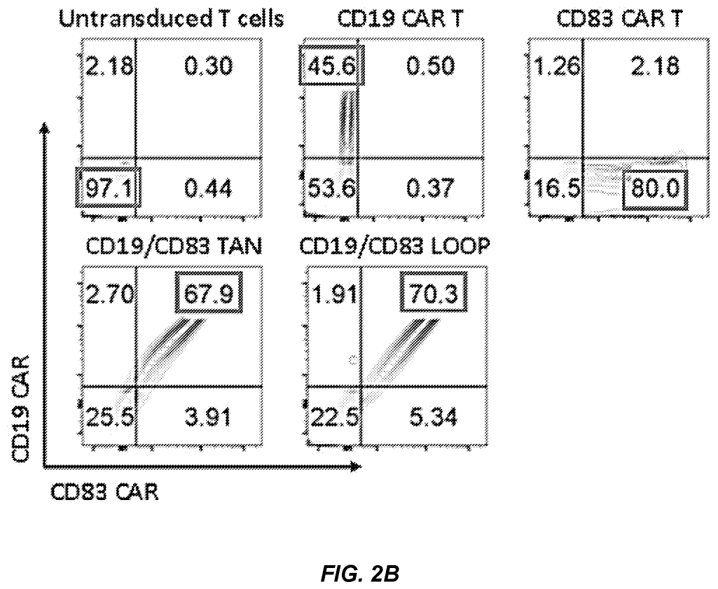

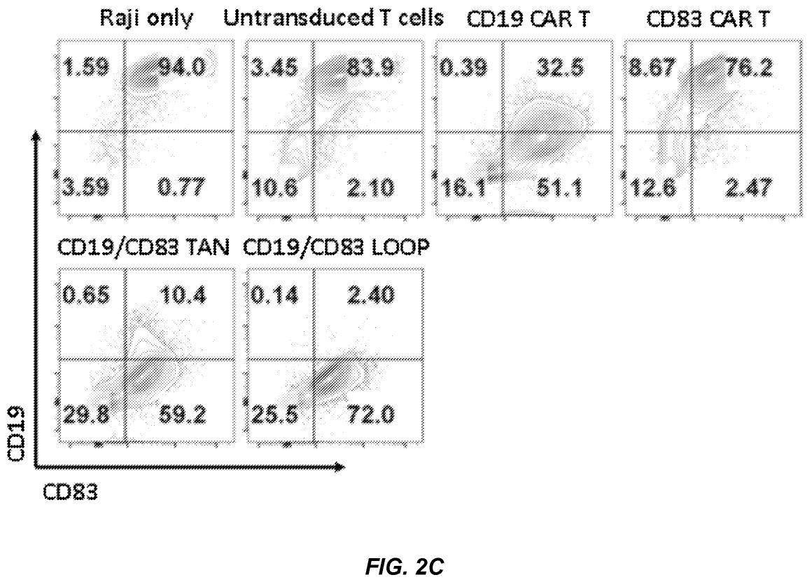

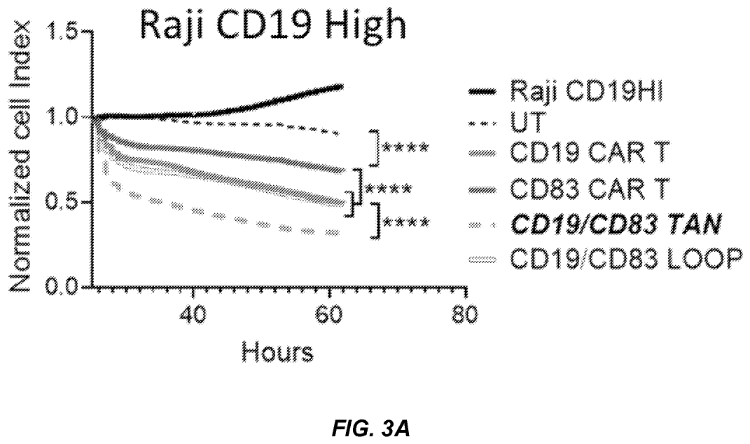

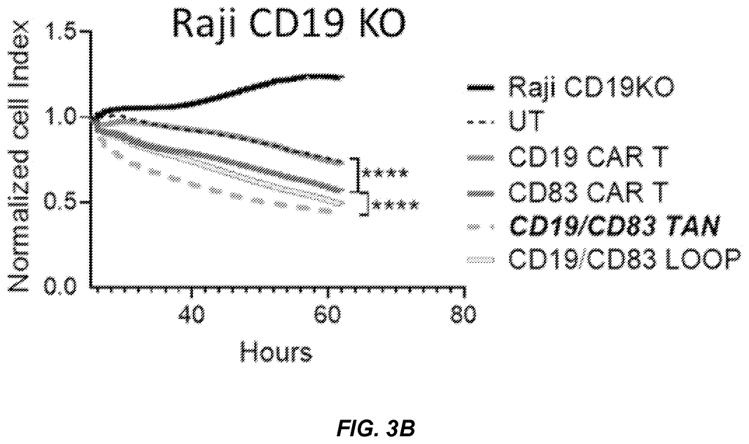

Development of ‘AND/OR’ logic gated CD19/CD83 CAR T. Rather than testing pooled, mono-gated CAR T, bispecific ‘OR’ logic gated CD19/CD83 CAR T were developed to maximize potency against ALL, overcome CD19 antigen escape, and prevent GVHD. T cells require several levels of activation for optimal cytolytic function. The disclosed ‘OR’ gated CD19/CD83 CAR T uses a loop or tandem, dual-scFv design (Qin H, et al. Mol Ther Oncolytics 2018 11:127-37), where engagement of either CD19 or CD83 leads to shared CD3ζ and 41BB (signals 1 and 2) T cell activation via a single endodomain (FIG. 2A). The loop and tandem designs spatially differ in their dual CAR surface expression on the T cell as shown in FIG. 2A (Qin H, et al. Mol Ther Oncolytics 2018 11:127-37), as the CD19 and CD83 scFvs are covalently linked in different orders. These spatial differences may prove to impact dual CD19/CD83 CAR T cytolytic activity against leukemia and lymphoma. Importantly, the ‘OR’ gated CD19/CD83 CAR T can potently kill target cells that express CD19 or CD83, and may achieve synergistic cytotoxicity if the antigens are co-expressed. As shown with the mono-gated CD83 CAR T for AML (Shrestha B, et al. J Clin Invest 2020), robust gammaretroviral transduction efficacy of the CD19/CD83 CAR constructs was achieved in human T cells (FIG. 2B). CD19 CAR T was also shown to lead to antigen loss, while CD83 expression remains stable even in the presence of either CD19 or CD83 CAR T (FIG. 2C). This supports the concept that targeting CD83 can overcome CD19 antigen loss with CAR T therapy. Others have developed CD19/CD20 or CD19/CD22 bispecific ‘OR’ gated CAR T for B cell malignancies (Spiegel J Y, et al. Nat Med 2021; Zhang Y, et al. Leukemia 2021). However, like CD19, CD22 is also susceptible to antigen escape (Schneider D, et al. Sci Transl Med 2021 13(586)). Interestingly, CD83 expression is similar to CD20 or CD22 across several B cell malignancies, but unlike CD20 or CD22, CD83 expression on circulating healthy B cells is negligible (FIG. 1A, preceding page). Using a Raji cell line transduced to express high levels of CD19 (MFI 1 log higher than wild type), the disclosed CD83 CAR T offers intermediate cytotoxicity compared to the CD19 CAR T (FIG. 3A). This is expected given the increased density of CD19 antigen compared to CD83. As proof-of-concept of anti-CD19/CD83 synergy, our ‘OR’ gated CD19/CD83 tandem CAR T offers enhanced killing of the Raji CD19^{HI} target cells, compared to CD19 or CD83 CAR T alone (FIG. 3A). The tandem CAR T also outperforms the loop construct (FIG. 3A), even though both constructs use identical anti-CD19 and anti-CD83 scFvs. This supports the concept that spatial interactions between tandem versus loop CAR constructs and their target antigens may impact resultant CAR T function. Mimicking CD19 antigen loss, we show CD83 CAR T significantly kill CD19 knock out Raji cells (˜1% CD19+), compared to CD19 CAR T (FIG. 3B). Moreover, the ‘OR’ gated CD19/CD83 CAR T (loop and tandem constructs) significantly kill Raji targets regardless of CD19 expression (FIG. 3A,B). Further, the tandem CD19/CD83 CAR T offers enhanced cytolytic potency against CD19 deficient Raji cells, compared to CD83 CAR T (FIG. 3B). Thus, the tandem bispecific CAR design may provide better activation than the mono-CAR, as both CAR T are targeting CD83 alone against the CD19KO Raji cells. This further supports the rationale to investigate and compare the tandem versus loop ‘OR’ gating constructs, the potential impact of spatial interactions between the CARs and their target antigens, and the activation characteristics of the bispecific CAR designs. Importantly, our proof-of-concept data (FIG. 9) support that ‘OR’ gated CD19/CD83 CAR T can provide enhanced cytolytic activity against CD19+/CD83+ lymphoma targets and overcome CD19 antigen escape, as CD83 expression is retained.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A dual-chimeric antigen receptor (CAR)-T cell, comprising an immune effector cell genetically modified to express a first CAR polypeptide that selectively binds CD83 and a second CAR polypeptide that selectively binds a second antigen selected from the group consisting of CD33, CLEC12A, CD123, CD38, and FLT3.

2. The dual-CAR-T cell of claim 1, wherein the first CAR polypeptide is defined by the formula:
SP-CD83-HG-TM-CSR, and
wherein the second CAR polypeptide is defined by the formula:
SP-TA-HG-TM-SD; or
wherein the first CAR polypeptide is defined by the formula:
SP-CD83-HG-TM-SD, and
wherein the second CAR polypeptide is defined by the formula:
SP-TA-HG-TM-CSR;
wherein “SP” represents an optional signal peptide,
wherein “CD83” represents a CD83-binding region,
wherein “TA” represents an CD33-binding region, CLEC12A-binding region, CD123-binding region, CD83-binding region, or FLT3-binding region,
wherein “HG” represents an optional hinge domain,
wherein “TM” represents a transmembrane domain,
wherein “CSR” represents one or more co-stimulatory signaling regions,
wherein “SD” represents a signaling domain, and
wherein “-” represents a peptide bond or linker.

3. The dual-CAR-T cell of claim 1, wherein the CD83 antigen binding domain is a single-chain variable fragment (scFv) of an antibody comprising a variable heavy (V_{H}) domain having CDR1, CDR2 and CDR3 sequences and a variable light (V_{L}) domain having CDR1, CDR2 and CDR3 sequences, and wherein
the CDR1 sequence of the V_{H} domain comprises the amino acid sequence GFSITTGGYWWT (SEQ ID NO:1), the CDR2 sequence of the V_{H} domain comprises the amino acid sequence GYIFSSGNTNYNPSIKS (SEQ ID NO:2), the CDR3 sequence of the V_{H} domain comprises the amino acid sequence CARAYGKLGFDY (SEQ ID NO:3), the CDR1 sequence of the V_{L} comprises the amino acid sequence TLSSQHSTYTIG (SEQ ID NO:4), the CDR2 sequence of the V_{L} domain comprises the amino acid sequence VNSDGSHSKGD (SEQ ID NO:5), and the CDR3 sequence of the V_{L} domain comprises the amino acid sequence GSSDSSGYV (SEQ ID NO:6);
the CDR1 sequence of the V_{H} domain comprises the amino acid sequence SDGIS (SEQ ID NO:7), the CDR2 sequence of the V_{H} domain comprises the amino acid sequence IISSGGNTYYASWAKG (SEQ ID NO:8), the CDR3 sequence of the V_{H} domain comprises the amino acid sequence VVGGTYSI (SEQ ID NO:9), the CDR1 sequence of the V_{L} comprises the amino acid sequence QSSQS VYNNDFLS (SEQ ID NO:10), the CDR2 sequence of the V_{L} domain comprises the amino acid sequence YASTLAS (SEQ ID NO:11), and the CDR3 sequence of the V_{L} domain comprises the amino acid sequence TGTYGNSAWYEDA (SEQ ID NO:12); or
the CDR1 sequence of the V_{H} domain comprises the amino acid sequence SNAMI (SEQ ID NO:13), the CDR2 sequence of the V_{H} domain comprises the amino acid sequence AMDSNSRTYYATWAKG (SEQ ID NO:14), the CDR3 sequence of the V_{H} domain comprises the amino acid sequence GDGGSSDYTEM (SEQ ID NO:15), the CDR1 sequence of the V_{L} comprises the amino acid sequence QSSQSVYGNNELS (SEQ ID NO:16), the CDR2 sequence of the V_{L} domain comprises the amino acid sequence QASSLAS (SEQ ID NO:17), and the CDR3 sequence of the V_{L} domain comprises the amino acid sequence LGEYSISADNH (SEQ ID NO:18).

4. The dual-CAR-T cell of claim 3, wherein the anti-CD83 scFv V_{L} domain comprises the amino acid sequence SEQ ID NO:39, SEQ ID NO:40, SEQ ID NO:41, SEQ ID NO:42, or SEQ ID NO:43.

5. The dual-CAR-T cell of claim 1, wherein the second CAR polypeptide comprises a CD33 antigen binding domain, wherein the CD33 antigen binding domain is a scFv of an antibody comprising a V_{H} domain having CDR1, CDR2 and CDR3 sequences and a V_{L} domain having CDR1, CDR2 and CDR3 sequences, and wherein
the CDR1 sequence of the V_{H} domain comprises the amino acid sequence GFTFSNYG (SEQ ID NO:72), the CDR2 sequence of the V_{H} domain comprises the amino acid sequence ISSGGGDT (SEQ ID NO:73), the CDR3 sequence of the V_{H} domain comprises the amino acid sequence ARDYGGTWDYFDY (SEQ ID NO:74); or
the CDR1 sequence of the V_{H} domain comprises the amino acid sequence GYTFTSYW (SEQ ID NO:75), the CDR2 sequence of the V_{H} domain comprises the amino acid sequence IHPSDSET (SEQ ID NO:76), the CDR3 sequence of the V_{H} domain comprises the amino acid sequence AREEGQLGHGGAMDY (SEQ ID NO:77); and
the CDR1 sequence of the V_{L} comprises the amino acid sequence QDISKY (SEQ ID NO:78), wherein the CDR2 sequence of the V_{L} domain comprises the amino acid sequence YTSx (SEQ ID NO:79), wherein the CDR3 sequence of the V_{L} domain comprises the amino acid sequence QQGDTFPWT (SEQ ID NO:80).

6. The dual-CAR-T cell of claim 5, wherein the anti-CD33 scFv V_{H} domain comprises the amino acid sequence SEQ ID NO:81 or SEQ ID NO:82.

7. The dual-CAR-T cell of claim 5, wherein the anti-CD33 scFv V_{L} domain comprises the amino acid sequence SEQ ID NO:83.

8. The dual-CAR-T cell of claim 5, wherein the anti-CD33 scFv V_{H} domain comprises the amino acid sequence SEQ ID NO:84.

9. The dual-CAR-T cell of claim 5, wherein the anti-CD33 scFv V_{L} domain comprises the amino acid sequence SEQ ID NO:85, SEQ ID NO:86, or SEQ ID NO:87.

10. The dual-CAR-T cell of claim 1, wherein the second CAR polypeptide comprises a CLEC12A antigen binding domain, wherein the CLEC12A antigen binding domain is a scFv of an antibody comprising a V_{H} domain having CDR1, CDR2 and CDR3 sequences and a V_{L} domain having CDR1, CDR2 and CDR3 sequences, and wherein
the CDR1 sequence of the V_{H} domain comprises the amino acid sequence GFTFSSFA (SEQ ID NO:93) or SFAVS (SEQ ID NO:94), CDR2 sequence of the V_{H} domain comprises the amino acid sequence ISSGGAYT (SEQ ID NO:95), CDR3 sequence of the V_{H} domain comprises the amino acid sequence HSGYDGYYLYAMDY (SEQ ID NO:96); or
the CDR1 sequence of the V_{H} domain comprises the amino acid sequence SHDMS (SEQ ID NO:97); the CDR2 sequence of the V_{H} domain comprises the amino acid sequence YISGGGTNIYYSDTVKGRFT (SEQ ID NO:98); the CDR3 sequence of the V_{H} domain comprises the amino acid sequence PNYNYGGSWFAY (SEQ ID NO:99); and
CDR1 sequence of the V_{L} comprises the amino acid sequence SSVHY (SEQ ID NO:100), CDR2 sequence of the V_{L} domain comprises the amino acid sequence DTSX (SEQ ID NO:101), and CDR3 sequence of the V_{L} domain comprises the amino acid sequence QQWTSNPPT (SEQ ID NO:102).

11. The dual-CAR-T cell of claim 8, wherein the anti-CLEC12A scFv V_{H} domain comprises the amino acid sequence SEQ ID NO:103, SEQ ID NO:104, or SEQ ID NO:105.

12. The dual-CAR-T cell of claim 10, wherein the anti-CLEC12A scFv V_{L} domain comprises the amino acid sequence SEQ ID NO:106.

13. The dual-CAR-T cell of claim 1, wherein the second CAR polypeptide comprises a CD123 antigen binding domain, wherein the CD123 antigen binding domain is a scFv of an antibody comprising a V_{H} domain having CDR1, CDR2 and CDR3 sequences and a V_{L} domain having CDR1, CDR2 and CDR3 sequences, and wherein the CDR1 sequence of the V_{H} domain comprises the amino acid sequence GYTFTDYN (SEQ ID NO:110), CDR2 sequence of the V_{H} domain comprises the amino acid sequence INPNNGGT (SEQ ID NO:111), CDR3 sequence of the V_{H} domain comprises the amino acid sequence ARKGYGGNYDYFDY (SEQ ID NO:112), CDR1 sequence of the V_{L} comprises the amino acid sequence QSIGTS (SEQ ID NO:113), CDR2 sequence of the V_{L} domain comprises the amino acid sequence YASx (SEQ ID NO:114), and CDR3 sequence of the V_{L} domain comprises the amino acid sequence QQSNSWPYT (SEQ ID NO:115).

14. The dual-CAR-T cell of claim 13, wherein the anti-CD123 scFv V_{H} domain comprises the amino acid sequence SEQ ID NO:143.

15. The dual-CAR-T cell of claim 13, wherein the anti-CD123 scFv V_{L} domain comprises the amino acid sequence SEQ ID NO:144.

16. The dual-CAR-T cell of claim 1, wherein the second CAR polypeptide comprises a CD123 antigen binding domain, wherein the CD123 antigen binding domain is a scFv of an antibody comprising a V_{H} domain having CDR1, CDR2 and CDR3 sequences and a V_{L} domain having CDR1, CDR2 and CDR3 sequences, and wherein the CDR1 sequence of the V_{H} domain comprises the amino acid sequence GFNIKDTY (SEQ ID NO:116) or GFSLSTYGMG (SEQ ID NO:117), the CDR2 sequence of the V_{H} domain comprises the amino acid sequence IDPANGNT (SEQ ID NO:118) or IYWDDDK (SEQ ID NO:119), the CDR3 sequence of the V_{H} domain comprises the amino acid sequence ALYYYGGSLDY (SEQ ID NO:120) or AQSLIYDGYYGFAY (SEQ ID NO:121), the CDR1 sequence of the V_{L} comprises the amino acid sequence QSLLYSGNQKNY (SEQ ID NO:122), the CDR2 sequence of the V_{L} domain comprises the amino acid sequence WASx (SEQ ID NO:123), and the CDR3 sequence of the V_{L} domain comprises the amino acid sequence QQYYSYPRT (SEQ ID NO:124).

17. The dual-CAR-T cell of claim 16, wherein the anti-CD123 scFv V_{H} domain comprises the amino acid sequence SEQ ID NO:145, SEQ ID NO:146, or SEQ ID NO:147.

18. The dual-CAR-T cell of claim 16, wherein the anti-CD123 scFv V_{L} domain comprises the amino acid sequence SEQ ID NO:148 or SEQ ID NO:149.

19. The dual-CAR-T cell of claim 1, wherein the second CAR polypeptide comprises a CD123 antigen binding domain, wherein the CD123 antigen binding domain is a scFv of an antibody comprising a V_{H} domain having CDR1, CDR2 and CDR3 sequences and a V_{L} domain having CDR1, CDR2 and CDR3 sequences, and wherein the CDR1 sequence of the V_{H} domain comprises the amino acid sequence GYTFSSYW (SEQ ID NO:131) or GYTLTTYL (SEQ ID NO:132), the CDR2 sequence of the V_{H} domain comprises the amino acid sequence INPSSGYT (SEQ ID NO:133) or INPNSGSS (SEQ ID NO:134), the CDR3 sequence of the V_{H} domain comprises the amino acid sequence ARDGNYDHWYFDV (SEQ ID NO:135) or AIRHYGGSLFDY (SEQ ID NO:136), the CDR1 sequence of the V_{L} comprises the amino acid sequence QDINSY (SEQ ID NO:137) or QSLLNSRTRKNY (SEQ ID NO:138), the CDR2 sequence of the V_{L} domain comprises the amino acid sequence WASx (SEQ ID NO:139), or RANx (SEQ ID NO:140), and the CDR3 sequence of the V_{L} domain comprises the amino acid sequence LQYDELLT (SEQ ID NO:141) or EQSYNLFT (SEQ ID NO:142).

20. The dual-CAR-T cell of claim 19, wherein the anti-CD123 scFv V_{H} domain comprises the amino acid sequence SEQ ID NO:150 or SEQ ID NO:151.

21. The dual-CAR-T cell of claim 19, wherein the anti-CD123 scFv V_{L} domain comprises the amino acid sequence SEQ ID NO:152 or SEQ ID NO:153.

22. The dual-CAR-T cell of claim 1, wherein the second CAR polypeptide comprises a CD38 antigen binding domain, wherein the CD38 antigen binding domain is a scFv of an antibody comprising a V_{H} domain having CDR1, CDR2 and CDR3 sequences and a V_{L} domain having CDR1, CDR2 and CDR3 sequences, and wherein the CDR1 sequence of the V_{H} domain comprises the amino acid sequence SFAMS (SEQ ID NO:162), CDR2 sequence of the V_{H} domain comprises the amino acid sequence AISGSGGGTYYADSVKG (SEQ ID NO:163), CDR3 sequence of the V_{H} domain comprises the amino acid sequence DKILWFGEPVFDY (SEQ ID NO:164), CDR 1 sequence of the VL comprises the amino acid sequence RASQSVSSYLA (SEQ ID NO:165), CDR2 sequence of the VL domain comprises the amino acid sequence DASNRAT (SEQ ID NO:166), and CDR3 sequence of the VL domain comprises the amino acid sequence QQRSNWPPTF (SEQ ID NO:167).

23. The dual-CAR-T cell of claim 22, wherein the anti-FLT3 scFv V_{H} domain comprises the amino acid sequence SEQ ID NO:168.

24. The dual-CAR-T cell of claim 22, wherein the anti-FLT3 scFv V_{L} domain comprises the amino acid sequence SEQ ID NO:169.

25. The dual-CAR-T cell of claim 1, wherein the second CAR polypeptide comprises a FLT3 antigen binding domain, wherein the FLT3 antigen binding domain is a scFv of an antibody comprising a V_{H} domain having CDR1, CDR2 and CDR3 sequences and a V_{L} domain having CDR1, CDR2 and CDR3 sequences, and wherein the CDR1 sequence of the V_{H} domain comprises the amino acid sequence SYWMH (SEQ ID NO:171), CDR2 sequence of the V_{H} domain comprises the amino acid sequence EIDPSDSYKDYNQKFKD (SEQ ID NO:172), CDR3 sequence of the V_{H} domain comprises the amino acid sequence AITTTPFDF (SEQ ID NO:173), CDR1 sequence of the VL comprises the amino acid sequence RASQSISNNLH (SEQ ID NO:174), CDR2 sequence of the VL domain comprises the amino acid sequence YASQSIS (SEQ ID NO:175), and CDR3 sequence of the VL domain comprises the amino acid sequence QQSNTWPYT (SEQ ID NO:176).

26. The dual-CAR-T cell of claim 22, wherein the anti-FLT3 scFv V_{H} domain comprises the amino acid sequence SEQ ID NO:177.

27. The dual-CAR-T cell of claim 22, wherein the anti-FLT3 scFv V_{L} domain comprises the amino acid sequence SEQ ID NO:178.

28. The dual-CAR-T cell of claim 1, wherein the second CAR polypeptide comprises a FLT3 antigen binding domain, wherein the FLT3 antigen binding domain is a scFv of an antibody comprising a V_{H} domain having CDR1, CDR2 and CDR3 sequences and a V_{L} domain having CDR1, CDR2 and CDR3 sequences, and wherein the CDR1 sequence of the V_{H} domain comprises the amino acid sequence NYGLH (SEQ ID NO:79), CDR2 sequence of the V_{H} domain comprises the amino acid sequence VIWSGGSTDYNAAFIS (SEQ ID NO:189101 CDR3 sequence of the V_{H} domain comprises the amino acid sequence GGIYYANHYYAMDY (SEQ ID NO:114), CDR1 sequence of the VL comprises the amino acid sequence KSSQSLLNSGNQKNYM (SEQ ID NO:123), CDR2 sequence of the VL domain comprises the amino acid sequence GASTRES (SEQ ID NO:129), and CDR3 sequence of the VL domain comprises the amino acid sequence QNDHSYPLT (SEQ ID NO:139).

29. The dual-CAR-T cell of claim 22, wherein the anti-FLT3 scFv V_{H} domain comprises the amino acid sequence SEQ ID NO:140.

30. The dual-CAR-T cell of claim 22, wherein the anti-FLT3 scFv V_{L} domain comprises the amino acid sequence SEQ ID NO:180.

31. The dual-CAR-T cell of claim 1, wherein the immune effector cell is selected from the group consisting of a T cell, a Natural Killer (NK) cell, a cytotoxic T lymphocyte (CTL), and a regulatory T cell.

32. A method of providing an anti-cancer immunity in a subject with a CD83-expressing B and/or T cell acute lymphoblastic leukemia (ALL), the method comprising administering to the subject an effective amount of the dual-CAR-T cell of claim 1, thereby providing an anti-tumor immunity in the mammal.

33. The method of claim 32, further comprising administering to the subject a checkpoint inhibitor.

34. The method of claim 33, wherein the checkpoint inhibitor comprises an anti-PD-1 antibody, anti-PD-L1 antibody, anti-CTLA-4 antibody, or a combination thereof.

35. The method of claim 32, wherein the cancer comprises an acute lymphoblastic leukemia (ALL) or acute myeloid leukemia (AML).


SEQUENCE LISTING (181 sequences)

SEQ ID NO: 1 - protein, 12 aa, unidentified
GFSITTGGYWWT

SEQ ID NO: 2 - protein, 17 aa, unidentified
GYIFSSGNTNYNPSIKS

SEQ ID NO: 3 - protein, 12 aa, unidentified
CARAYGKLGFDY

SEQ ID NO: 4 - protein, 12 aa, unidentified
TLSSQHSTYTIG

SEQ ID NO: 5 - protein, 11 aa, unidentified
VNSDGSHSKGD

SEQ ID NO: 6 - protein, 9 aa, unidentified
GSSDSSGYV

SEQ ID NO: 7 - protein, 5 aa, unidentified
SDGIS

SEQ ID NO: 8 - protein, 16 aa, unidentified
IISSGGNTYYASWAKG

SEQ ID NO: 9 - protein, 8 aa, unidentified
VVGGTYSI

SEQ ID NO: 10 - protein, 13 aa, unidentified
QSSQSVYNNDFLS

SEQ ID NO: 11 - protein, 7 aa, unidentified
YASTLAS

SEQ ID NO: 12 - protein, 13 aa, unidentified
TGTYGNSAWYEDA

SEQ ID NO: 13 - protein, 5 aa, unidentified
SNAMI

SEQ ID NO: 14 - protein, 16 aa, unidentified
AMDSNSRTYYATWAKG

SEQ ID NO: 15 - protein, 11 aa, unidentified
GDGGSSDYTEM

SEQ ID NO: 16 - protein, 13 aa, unidentified
QSSQSVYGNNELS

SEQ ID NO: 17 - protein, 7 aa, unidentified
QASSLAS

SEQ ID NO: 18 - protein, 11 aa, unidentified
LGEYSISADNH

SEQ ID NO: 19 - protein, 120 aa, unidentified
QVQLKESGPGLVKPSQSLSLTCSVTGFSITTGGYWWTWIRQFPGQKLEWMGYIFSSGNTNYNPSIKSRISITRDTSKNQFFLQLNSVTTEGDTARYYCARAYGKLGFDYWGQGTLVTVSS

SEQ ID NO: 20 - protein, 111 aa, unidentified
QPVLTQSPSASASLGNSVKITCTLSSQHSTYTIGWYQQHPDKAPKYVMYVNSDGSHSKGDGIPDRFSGSSSGAHRYLSISNIQPEDEADYFCGSSDSSGYVFGSGTQLTVL

SEQ ID NO: 21 - protein, 454 aa, unidentified
METGLRWLLLVAVLKGVQCQSVEESGGRLVTPGTPLTLTCTVSGFSLSNNAINWVRQAPGKGLEWIGYIWSGGLTYYANWAEGRFTISKTSTTVDLKMTSPTIEDTATYFCARGINNSALWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYNKLSVPTSEWQRGDVFTCSVMHEALHNHYTQKSISRSPGK

SEQ ID NO: 22 - protein, 239 aa, unidentified
MDMRAPTQLLGLLLLWLPGARCADVVMTQTPASVSAAVGGTVTINCQASESISNYLSWYQQKPGQPPKLLIYRTSTLASGVSSRFKGSGSGTEYTLTISGVQCDDVATYYCQCTSGGKFISDGAAFGGGTEVVVKGDPVAPTVLLFPPSSDEVATGTVTIVCVANKYFPDVTVTWEVDGTTQTTGIENSKTPQNSADCTYNLSSTLTLTSTQYNSHKEYTCKVTQGTTSVVQSFSRKNC

SEQ ID NO: 23 - protein, 452 aa, unidentified
METGLRWLLLVAVLKGVQCQSVEESGGRLVTPGTPLTLTCTVSGFTISDYDLSWVRQAPGEGLKYIGFIAIDGNPYYATWAKGRFTISKTSTTVDLKITAPTTEDTATYFCARGAGDLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYNKLSVPTSEWQRGDVFTCSVMHEALHNHYTQKSISRSPGK

SEQ ID NO: 24 - protein, 238 aa, unidentified
MDTREPTQLLGLLLLWLPGARCADVVMTQTPASVSAAVGGTVTINCQSSKNVYNNNWLSWFQQKPGQPPKLLIYYASTLASGVPSRFRGSGSGTQFTLTISDVQCDDAATYYCAGDYSSSSDNGFGGGTEVVVKGDPVAPTVLLFPPSSDEVATGTVTIVCVANKYFPDVTVTWEVDGTTQTTGIENSKTPQNSADCTYNLSSTLTLTSTQYNSHKEYTCKVTQGTTSVVQSFSRKNC

SEQ ID NO: 25 - protein, 454 aa, unidentified
METGLRWLLLVAVLKGVHCQSVEESGGRLVTPGTPLTLTCTASGFSRSSYDMSWVRQAPGKGLEWVGVISTAYNSHYASWAKGRFTISRTSTTVDLKMTSLTTEDTATYFCARGGSWLDLWGQGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYNKLSVPTSEWQRGDVFTCSVMHEALHNHYTQKSISRSPGK

SEQ ID NO: 26 - protein, 239 aa, unidentified
MDXRAPTQLLGLLLLWLPGARCALVMTQTPASVSAAVGGTVTINCQSSQSVYDNDELSWYQQKPGQPPKLLIYALASKLASGVPSRFKGSGSGTQFALTISGVQCDDAATYYCQATHYSSDWYLTFGGGTEVVVKGFPVAPTVLLFPPSSDEVATGTVTIVCVANKYFPDVTVTWEVDGTTQTTGTENSKTPQNSADCTYNLSSTLTLTSTQYNSHKEYTCKVTQGTTSVVQSFSRKNC

SEQ ID NO: 27 - protein, 456 aa, unidentified
METGLRWLLLVAVLKGVQCQSVEESGGRLVTPGTPLTLTCTVSGFSLSSYDMTWVRQAPGKGLEWIGIIYASGTTYYANWAKGRFTISKTSTTVDLKVTSPTIGDTATYFCAREGAGVSMTLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYNKLSVPTSEWQRGDVFTCSVMHEALHNHYTQKSISRSPGK

SEQ ID NO: 28 - protein, 236 aa, unidentified
MDMRAPTQLLGLLLLWLPGARCAYDMTQTPASVEVAVGGTVTIKCQASQSISTYLDWYQQKPGQPPKLLIYDASDLASGVPSRFKGSGSGTQFTLTISDLECADAATYYCQQGYTHSNVDNVFGGGTEVVVKGDPVAPTVLLFPPSSDEVATGTVTIVCVANKYFPDVTVTWEVDGTTQTTGIENSKTPQNSADCTYNLSSTLTLTSTQYNSHKEYTCKVTQGTTSVVQSFSRKNC

SEQ ID NO: 29 - protein, 459 aa, unidentified
METGLRWLLLVAVLKGVQCQSVEESGGRLVSPGTPLTLTCTASGFSLSSYDMSWVRQAPGKGLEYIGIISSSGSTYYASWAKGRFTISKTSTTVDLEVTSLTTEDTATYFCSREHAGYSGDTGHLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVGIGPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYNKLSVPTSEWQRGDVFTCSVMHEALHNHYTQKSISRSPGK

SEQ ID NO: 30 - protein, 236 aa, unidentified
MDMRAPTQLLGLLLLWLPGARCAYDMTQTPASVEVAVGGTVAIKCQASQSVSSYLAWYQQKPGQPPKPLIYEASMLAAGVSSRFKGSGSGTDFTLTISDLECDDAATYYCQQGYSISDIDNAFGGGTEVVVKGDPVAPTVLLFPPSSDEVATGTVTIVCVANKYFPDVTVTWEVDGTTQTTGIENSKTPQNSADCTYNLSSTLTLTSTQYNSHKEYTCKVTQGTTSVVQSFSRKNC

SEQ ID NO: 31 - protein, 455 aa, unidentified
METGLRWLLLVAVLKGVQCQSVEESGGRLVTPGTPLTLTCTVSGIDLSSDGISWVRQAPGKGLEWIGIISSGGNTYYASWAKGRFTISRTSTTVDLKMTSLTTEDTATYFCARVVGGTYSIWGQGTLVTVSSASTKGPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 32 - protein, 240 aa, unidentified
MDTRAPTQLLGLLLLWLPGATFAQVLTQTASPVSAPVGGTVTINCQSSQSVYNNDFLSWYQQKPGQPPKLLIYYASTLASGVPSRFKGSGSGTQFTLTISDLECDDAATYYCTGTYGNSAWYEDAFGGGTEVVVKRTPVAPTVLLFPPSSAELATGTATIVCVANKYFPDGTVTWKVDGITQSSGINNSRTPQNSADCTYNLSSTLTLSSDEYNSHDEYTCQVAQDSGSPVVQSFSRKSC

SEQ ID NO: 33 - protein, 460 aa, unidentified
METGLRWLLLVAVLKGVQCQSVEESGGRLVTPGTPLTLTCTVSGIDLSSNAMIWVRQAPREGLEWIGAMDSNSRTYYATWAKGRFTISRTSSITVDLKITSPTTEDTATYFCARGDGGSSDYTEMWGPGTLVTVSSASTKGPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 34 - protein, 239 aa, unidentified
MDTRAPTQLLGLLLLWLPGATFAQAVVTQTTSPVSAPVGGTVTINCQSSQSVYGNNELSWYQQKPGQPPKLLIYQASSLASGVPSRFKGSGSGTQFTLTISDLECDDAATYYCLGEYSISADNHFGGGTEVVVKRTPVAPTVLLFPPSSAELATGTATIVCVANKYFPDGTVTWKVDGITQSSGINNSRTPQNSADCTYNLSSTLTLSSDEYNSHDEYTCQVAQDSGSPVVQSFSRKSC

SEQ ID NO: 35 - protein, 221 aa, unidentified
QVQLVQSGGAVVQPGRSLRLSCAASGFTFSTYGMHWVRQAPGKGLEWVAAVSYDGSNKYYADFVKGRFTISRDNPKNTLYLQMNSLRADDTAVYYCARRGGLDIWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCAAA

SEQ ID NO: 36 - protein, 110 aa, unidentified
LTQPPPASGTPGQQRVTISCSGSSSNIGSNTVNWYQQLPGTAPKLLIYYGNDQRPSGVPDRFSASKSGTSASLAISGLQSEDEAHYYCAAWDGSLNGGVIFGGGTKVTLG

SEQ ID NO: 37 - protein, 109 aa, unidentified
VTQPPSASGTPGQRVTISCSGSSSNIGTNPVNWYQQLPGTAPKLLIYTTDQRPSGVPDRFSGSKSGTSASLAISGLQSEDEADYYCAAWDDSLSGLYVFGTGTKVTVLG

SEQ ID NO: 38 - protein, 109 aa, unidentified
MTHTPLSLSVTPGQPASISCKSSQSLLHSDGKTYLYWYLQRPGQSPQPLIYEVSNRFSGVPDRFSGSGSGTDFTLKISRVQAEDVGVYYCMQSLQLWTFGQGTKVEIKR

SEQ ID NO: 39 - protein, 110 aa, unidentified
MTQSPLSLPVTLGQPASISCRSSQSLIHSDGNTYLDWFQQRPGQSPRRLIYKVSNRDSGVPDRFSGSGSGTDFTLRISRVEAEDIGVYYCMQATHWPRTFGQGTKVEIKR

SEQ ID NO: 40 - protein, 110 aa, unidentified
MTQSPLSLPVTLGQPASISCRSSQSLVDSAGNTFLHWFHQRPGQSPRRLIYKVSNRDSGVPDRFSGSGSGTDFTLKISRVEAEDVGVYYCMQGTHWPRTFGQGTKVEIKR

SEQ ID NO: 41 - protein, 110 aa, unidentified
LTQSPLSLPVTLGQPASISCKSSQSLVDSDGNTYLNWFQQRPGQSPRRLIYKVSNRDSGVPDRFSGSGSGTDFTLKISRVEAEDVGVYYCMQGTHWPRTFGQGTKVEIKR

SEQ ID NO: 42 - protein, 110 aa, unidentified
MTQSPLSLPVTLGQPASISCRSSQSLVHSDGNMYLNWFQQRPGQSPRRLIYKVSNRDSGVPDRFSGSGSGTDFTLKISRVEAEDVGVYYCMQATQPTWTFGQGTKLEIKR

SEQ ID NO: 43 - protein, 92 aa, unidentified
MTQSPSSLSASVGDRVTITCQASQDISNYLNWYQQKPGKAPKLLIYDASNLETGVPSRFSGSGSGTDFTFTISSATYYCQQTYQGTKLEIKR

SEQ ID NO: 44 - protein, 105 aa, unidentified
MTQSPSSLSASVGHPVTITCRASQSLISYLNWYHQKPGKAPKLLIYAASILQSGVPSRFSGSGSGTDFTLTISSLQPENFASYYCQHTDSFPRTFGHGTKVEIKR

SEQ ID NO: 45 - protein, 108 aa, unidentified
LTQPPSASGTPGQGVTISCRGSTSNIGNNVVNWYQHVPGSAPKLLIWSNIQRPSGIPDRFSGSKSGTSASLAISGLQSEDQAVYYCAVWDDGLAGWVFGGGTTVTVLS

SEQ ID NO: 46 - protein, 105 aa, unidentified
MTQAPVVSVALEQTVRITCQGDSLAIYYDFWYQHKPGQAPVLVIYGKNNRPSGIPHRFSGSSSNTDSLTITGAQAEDEADYYCNSRDSSGNHWVFGGGTNLTVLG

SEQ ID NO: 47 - protein, 111 aa, unidentified
LTQSPLSLPVTLGQPASISCKSNQSLVHSDGNTYLNWFQQRPGQSPRRLIYKVSNRDSGVPDRFSGSGSGTDFTLKINRVEAEDVGVYYCMQGTQWPRTFGGQGTKLDIKR

SEQ ID NO: 48 - protein, 119 aa, unidentified
QVQLQESGPGLVKPSETLSLTCTVSGFSITTGGYWWTWIRQPPGKGLEWIGYIFSSGNTNYNPSIKSRVTISVDTSKNQFSLKLSSVTAADTAVYYCARAYGKLGFDYWGQGTLVTVSS

SEQ ID NO: 49 - protein, 119 aa, unidentified
QVQLQESGPGLVKPSQTLSLTCTVSGFSITTGGYWWTWIRQHPGKGLEWIGYIFSSGNTNYNPSIKSLVTISVDTSKNQFSLKLSSVTAADTAVYYCARAYGKLGFDYWGQGTLVTVSS

SEQ ID NO: 50 - protein, 119 aa, unidentified
QVQLQESGPGLVKPSQTLSLTCTVSGFSITTGGYWWTWIRQPPGKGLEWIGYIFSSGNTNYNPSIKSRVTISVDTSKNQFSLKLSSVTAADTAVYYCARAYGKLGFDYWGQGTLVTVSS

SEQ ID NO: 51 - protein, 119 aa, unidentified
QVQLQESGPGLVKPSETLSLTCTVSGFSITTGGYWWTWIRQPPGKGLEWIGYIFSSGNTNYNPSIKSRVTISRDTSKNQFSLKLSSVTAADTAVYYCARAYGKLGFDYWGQGTLVTVSS

SEQ ID NO: 52 - protein, 119 aa, unidentified
QVQLQESGPGLVKPSETLSLTCTVSGFSITTGGYWWTWIRQPPGKGLEWIGYIFSSGNTNYNPSIKSRVTISVDTSKNQFSLKLSSVTAADTARYYCARAYGKLGFDYWGQGTLVTVSS

SEQ ID NO: 53 - protein, 120 aa, unidentified
QVQLQESGPGLVKPSETLSLTCTVSGFSITTGGYWWTWIRQPPGKGLEWIGYIFSSGNTNYNPSIKSRISITRDTSKNQFFLQLNSVTTEGDTARYYCARAYGKLGFDYWGQGTLVTVSS

SEQ ID NO: 54 - protein, 111 aa, unidentified
QLVLTQSPSASASLGASVKLTCTLSSQHSTYTIGWHQQQPEKGPRYLMKVNSDGSHSKGDGIPDRFSGSSSGAERYLTISSLQSEDEADYYCGSSDSSGYVFGSGTKVTVL

SEQ ID NO: 55 - protein, 111 aa, unidentified
LPVLTQPPSASALLGASIKLTCTLSSQHSTYTIGWYQQRPGRSPQYIMKVNSDGSHSKGDGIPDRFMGSSSGADRYLTFSNLQSDDEAEYHCGSSDSSGYVFGSGTKVTVL

SEQ ID NO: 56 - protein, 15 aa, unidentified
GGGGSGGGGSGGGGS

SEQ ID NO: 57 - protein, 247 aa, unidentified
QPVLTQSPSASASLGNSVKITCTLSSQHSTYTIGWYQQHPDKAPKYVMYVNSDGSHSKGDGIPDRFSGSSSGAHRYLSISNIQPEDEADYFCGSSDSSGYVFGSGTQLTVLRAAASSGGGGSGGGGSGGGGSQPVLTQSPSASASLGNSVKITCTLSSQHSTYTIGWYQQHPDKAPKYVMYVNSDGSHSKGDGIPDRFSGSSSGAHRYLSISNIQPEDEADYFCGSSDSSGYVFGSGTQLTVLRAAA

SEQ ID NO: 58 - protein, 253 aa, unidentified
QVQLKESGPGLVKPSQSLSLTCSVTGFSITTGGYWWTWIRQFPGQKLEWMGYIFSSGNTNYNPSIKSRISITRDTSKNQFFLQLNSVTTEGDTARYYCARAYGKLGFDYWGQGTLVTVSSGGGGSGGGGSGGGGSQVQLKESGPGLVKPSQSLSLTCSVTGFSITTGGYWWTWIRQFPGQKLEWMGYIFSSGNTNYNPSIKSRISITRDTSKNQFFLQLNSVTTEGDTARYYCARAYGKLGFDYWGQGTLVTV

SEQ ID NO: 59 - protein, 245 aa, unidentified
QVQLQESGPGLVKPSETLSLTCTVSGFSITTGGYWWTWIRQPPGKGLEWIGYIFSSGNTNYNPSIKSRVTISVDTSKNQFSLKLSSVTAADTAVYYCARAYGKLGFDYWGQGTLVTVSSGGGGSGGGGSGGGGSQLVLTQSPSASASLGASVKLTCTLSSQHSTYTIGWHQQQPEKGPRYLMKVNSDGSHSKGDGIPDRFSGSSSGAERYLTISSLQSEDEADYYCGSSDSSGYVFGSGTKVTVL

SEQ ID NO: 60 - protein, 245 aa, unidentified
QVQLQESGPGLVKPSQTLSLTCTVSGFSITTGGYWWTWIRQHPGKGLEWIGYIFSSGNTNYNPSIKSLVTISVDTSKNQFSLKLSSVTAADTAVYYCARAYGKLGFDYWGQGTLVTVSSGGGGSGGGGSGGGGSQLVLTQSPSASASLGASVKLTCTLSSQHSTYTIGWHQQQPEKGPRYLMKVNSDGSHSKGDGIPDRFSGSSSGAERYLTISSLQSEDEADYYCGSSDSSGYVFGSGTKVTVL

SEQ ID NO: 61 - protein, 245 aa, unidentified
QVQLQESGPGLVKPSQTLSLTCTVSGFSITTGGYWWTWIRQPPGKGLEWIGYIFSSGNTNYNPSIKSRVTISVDTSKNQFSLKLSSVTAADTAVYYCARAYGKLGFDYWGQGTLVTVSSGGGGSGGGGSGGGGSQLVLTQSPSASASLGASVKLTCTLSSQHSTYTIGWHQQQPEKGPRYLMKVNSDGSHSKGDGIPDRFSGSSSGAERYLTISSLQSEDEADYYCGSSDSSGYVFGSGTKVTVL

SEQ ID NO: 62 - protein, 245 aa, unidentified
QVQLQESGPGLVKPSETLSLTCTVSGFSITTGGYWWTWIRQPPGKGLEWIGYIFSSGNTNYNPSIKSRVTISRDTSKNQFSLKLSSVTAADTAVYYCARAYGKLGFDYWGQGTLVTVSSGGGGSGGGGSGGGGSQLVLTQSPSASASLGASVKLTCTLSSQHSTYTIGWHQQQPEKGPRYLMKVNSDGSHSKGDGIPDRFSGSSSGAERYLTISSLQSEDEADYYCGSSDSSGYVFGSGTKVTVL

SEQ ID NO: 63 - protein, 245 aa, unidentified
QVQLQESGPGLVKPSETLSLTCTVSGFSITTGGYWWTWIRQPPGKGLEWIGYIFSSGNTNYNPSIKSRVTISVDTSKNQFSLKLSSVTAADTARYYCARAYGKLGFDYWGQGTLVTVSSGGGGSGGGGSGGGGSQLVLTQSPSASASLGASVKLTCTLSSQHSTYTIGWHQQQPEKGPRYLMKVNSDGSHSKGDGIPDRFSGSSSGAERYLTISSLQSEDEADYYCGSSDSSGYVFGSGTKVTVL

SEQ ID NO: 64 - protein, 246 aa, unidentified
QVQLQESGPGLVKPSETLSLTCTVSGFSITTGGYWWTWIRQPPGKGLEWIGYIFSSGNTNYNPSIKSRISITRDTSKNQFFLQLNSVTTEGDTARYYCARAYGKLGFDYWGQGTLVTVSSGGGGSGGGGSGGGGSQLVLTQSPSASASLGASVKLTCTLSSQHSTYTIGWHQQQPEKGPRYLMKVNSDGSHSKGDGIPDRFSGSSSGAERYLTISSLQSEDEADYYCGSSDSSGYVFGSGTKVTVL

SEQ ID NO: 65 - protein, 245 aa, unidentified
QVQLQESGPGLVKPSETLSLTCTVSGFSITTGGYWWTWIRQPPGKGLEWIGYIFSSGNTNYNPSIKSRVTISVDTSKNQFSLKLSSVTAADTAVYYCARAYGKLGFDYWGQGTLVTVSSGGGGSGGGGSGGGGSLPVLTQPPSASALLGASIKLTCTLSSQHSTYTIGWYQQRPGRSPQYIMKVNSDGSHSKGDGIPDRFMGSSSGADRYLTFSNLQSDDEAEYHCGSSDSSGYVFGSGTKVTVL

SEQ ID NO: 66 - protein, 245 aa, unidentified
QVQLQESGPGLVKPSQTLSLTCTVSGFSITTGGYWWTWIRQHPGKGLEWIGYIFSSGNTNYNPSIKSLVTISVDTSKNQFSLKLSSVTAADTAVYYCARAYGKLGFDYWGQGTLVTVSSGGGGSGGGGSGGGGSLPVLTQPPSASALLGASIKLTCTLSSQHSTYTIGWYQQRPGRSPQYIMKVNSDGSHSKGDGIPDRFMGSSSGADRYLTFSNLQSDDEAEYHCGSSDSSGYVFGSGTKVTVL

SEQ ID NO: 67 - protein, 245 aa, unidentified
QVQLQESGPGLVKPSQTLSLTCTVSGFSITTGGYWWTWIRQPPGKGLEWIGYIFSSGNTNYNPSIKSRVTISVDTSKNQFSLKLSSVTAADTAVYYCARAYGKLGFDYWGQGTLVTVSSGGGGSGGGGSGGGGSLPVLTQPPSASALLGASIKLTCTLSSQHSTYTIGWYQQRPGRSPQYIMKVNSDGSHSKGDGIPDRFMGSSSGADRYLTFSNLQSDDEAEYHCGSSDSSGYVFGSGTKVTVL

SEQ ID NO: 68 - protein, 245 aa, unidentified
QVQLQESGPGLVKPSETLSLTCTVSGFSITTGGYWWTWIRQPPGKGLEWIGYIFSSGNTNYNPSIKSRVTISRDTSKNQFSLKLSSVTAADTAVYYCARAYGKLGFDYWGQGTLVTVSSGGGGSGGGGSGGGGSLPVLTQPPSASALLGASIKLTCTLSSQHSTYTIGWYQQRPGRSPQYIMKVNSDGSHSKGDGIPDRFMGSSSGADRYLTFSNLQSDDEAEYHCGSSDSSGYVFGSGTKVTVL

SEQ ID NO: 69 - protein, 245 aa, unidentified
QVQLQESGPGLVKPSETLSLTCTVSGFSITTGGYWWTWIRQPPGKGLEWIGYIFSSGNTNYNPSIKSRVTISVDTSKNQFSLKLSSVTAADTARYYCARAYGKLGFDYWGQGTLVTVSSGGGGSGGGGSGGGGSLPVLTQPPSASALLGASIKLTCTLSSQHSTYTIGWYQQRPGRSPQYIMKVNSDGSHSKGDGIPDRFMGSSSGADRYLTFSNLQSDDEAEYHCGSSDSSGYVFGSGTKVTVL

SEQ ID NO: 70 - protein, 246 aa, unidentified
QVQLQESGPGLVKPSETLSLTCTVSGFSITTGGYWWTWIRQPPGKGLEWIGYIFSSGNTNYNPSIKSRISITRDTSKNQFFLQLNSVTTEGDTARYYCARAYGKLGFDYWGQGTLVTVSSGGGGSGGGGSGGGGSLPVLTQPPSASALLGASIKLTCTLSSQHSTYTIGWYQQRPGRSPQYIMKVNSDGSHSKGDGIPDRFMGSSSGADRYLTFSNLQSDDEAEYHCGSSDSSGYVFGSGTKVTVL

SEQ ID NO: 71 - skipped

SEQ ID NO: 72 - protein, 8 aa, unidentified
GFTFSNYG

SEQ ID NO: 73 - protein, 8 aa, unidentified
ISSGGGDT

SEQ ID NO: 74 - protein, 13 aa, unidentified
ARDYGGTWDYFDY

SEQ ID NO: 75 - protein, 8 aa, unidentified
GYTFTSYW

SEQ ID NO: 76 - protein, 8 aa, unidentified
IHPSDSET

SEQ ID NO: 77 - protein, 15 aa, unidentified
AREEGQLGHGGAMDY

SEQ ID NO: 78 - protein, 6 aa, unidentified
QDISKY

SEQ ID NO: 79 - skipped

SEQ ID NO: 80 - protein, 9 aa, unidentified
QQGDTFPWT

SEQ ID NO: 81 - protein, 120 aa, unidentified
EVKLVESGGGLVKPGASLKLSCAASGFTFSNYGMSWVRQTSDKRLEWVASISSGGGDTYYPDNVKGRFTISRENAKNTLYLQMSSLNSEDTALYYCARDYGGTWDYFDYWGQGTTLTVSS

SEQ ID NO: 82 - protein, 122 aa, unidentified
QVQLQQPGAELVRPGVSVKLSCKASGYTFTSYWMNWVKQRPGQGLEWIGMIHPSDSETRLNQKFKDKAILTVDKSSSTAYMQLSSPTSEDSAVYYCAREEGQLGHGGAMDYWGQGTSVTVSS

SEQ ID NO: 83 - protein, 107 aa, unidentified
DIQMTQTTSSLSASLGDRVTISCRASQDISKYLNWYQQKPDGTVKLLIYYTSRLHSGVPSRFSGSGSGTDYSLTISNLEQEDIATYFCQQGDTFPWTFGGGTKLEIK

SEQ ID NO: 84 - protein, 117 aa, unidentified
QVQLKESGPGLVAPSQSLSITCTVSGFSLSRYSVHWVRQPPGKGLEWLGMIWGGGYTDYNSALKSRLSISKDNSKSQVFLKMNSLQTDDTAMYYCARYIDSSGYDYWGQGTTLTVSS

SEQ ID NO: 85 - protein, 107 aa, unidentified
SIVMTQTPKFLLVSAGDRVTITCKASQTVNDDVAWYQQKPGQSPKLLIYYVSNRHTGVPDRFTGSGYGTDFTFTISTVQAEDLAVYFCQQDYSSPYTFGGGTKLEIK

SEQ ID NO: 86 - protein, 106 aa, unidentified
QIVLTQSPAIMSASPGEKVTMTCSASSSVSYMHWYQQKSGTSPKRWIYDTSKLASGVPARFSGSGSGTSYSLTISSMEAEDAATYYCQQWSSNPLTFGAGTKLELK

SEQ ID NO: 87 - protein, 107 aa, unidentified
DIQMTQSPASLSASVGETVTITCRASENIYSYLAWYQQKQGKSPQLLVYNAKTLAEGVPSRFSGSGSGTQFSLKINSLQPEDFGSYYCQHHYGTPYTFGGGTKLEIK

SEQ ID NO: 88 - protein, 242 aa, unidentified
EVKLVESGGGLVKPGASLKLSCAASGFTFSNYGMSWVRQTSDKRLEWVASISSGGGDTYYPDNVKGRFTISRENAKNTLYLQMSSLNSEDTALYYCARDYGGTWDYFDYWGQGTTLTVSSGGGGSGGGGSGGGGSDIQMTQTTSSLSASLGDRVTISCRASQDISKYLNWYQQKPDGTVKLLIYYTSRLHSGVPSRFSGSGSGTDYSLTISNLEQEDIATYFCQQGDTFPWTFGGGTKLEIK

SEQ ID NO: 89 - protein, 244 aa, unidentified
QVQLQQPGAELVRPGVSVKLSCKASGYTFTSYWMNWVKQRPGQGLEWIGMIHPSDSETRLNQKFKDKAILTVDKSSSTAYMQLSSPTSEDSAVYYCAREEGQLGHGGAMDYWGQGTSVTVSSGGGGSGGGGSGGGGSDIQMTQTTSSLSASLGDRVTISCRASQDISKYLNWYQQKPDGTVKLLIYYTSRLHSGVPSRFSGSGSGTDYSLTISNLEQEDIATYFCQQGDTFPWTFGGGTKLEIK

SEQ ID NO: 90 - protein, 239 aa, unidentified
QVQLKESGPGLVAPSQSLSITCTVSGFSLSRYSVHWVRQPPGKGLEWLGMIWGGGYTDYNSALKSRLSISKDNSKSQVFLKMNSLQTDDTAMYYCARYIDSSGYDYWGQGTTLTVSSGGGGSGGGGSGGGGSSIVMTQTPKFLLVSAGDRVTITCKASQTVNDDVAWYQQKPGQSPKLLIYYVSNRHTGVPDRFTGSGYGTDFTFTISTVQAEDLAVYFCQQDYSSPYTFGGGTKLEIK

SEQ ID NO: 91 - protein, 238 aa, unidentified
QVQLKESGPGLVAPSQSLSITCTVSGFSLSRYSVHWVRQPPGKGLEWLGMIWGGGYTDYNSALKSRLSISKDNSKSQVFLKMNSLQTDDTAMYYCARYIDSSGYDYWGQGTTLTVSSGGGGSGGGGSGGGGSQIVLTQSPAIMSASPGEKVTMTCSASSSVSYMHWYQQKSGTSPKRWIYDTSKLASGVPARFSGSGSGTSYSLTISSMEAEDAATYYCQQWSSNPLTFGAGTKLELK

SEQ ID NO: 92 - protein, 239 aa, unidentified
QVQLKESGPGLVAPSQSLSITCTVSGFSLSRYSVHWVRQPPGKGLEWLGMIWGGGYTDYNSALKSRLSISKDNSKSQVFLKMNSLQTDDTAMYYCARYIDSSGYDYWGQGTTLTVSSGGGGSGGGGSGGGGSDIQMTQSPASLSASVGETVTITCRASENIYSYLAWYQQKQGKSPQLLVYNAKTLAEGVPSRFSGSGSGTQFSLKINSLQPEDFGSYYCQHHYGTPYTFGGGTKLEIK

SEQ ID NO: 93 - protein, 8 aa, unidentified
GFTFSSFA

SEQ ID NO: 94 - protein, 5 aa, unidentified
SFAVS

SEQ ID NO: 95 - protein, 8 aa, unidentified
ISSGGAYT

SEQ ID NO: 96 - protein, 14 aa, unidentified
HSGYDGYYLYAMDY

SEQ ID NO: 97 - protein, 5 aa, unidentified
SHDMS

SEQ ID NO: 98 - protein, 20 aa, unidentified
YISGGGTNIYYSDTVKGRFT

SEQ ID NO: 99 - protein, 12 aa, unidentified
PNYNYGGSWFAY

SEQ ID NO: 100 - protein, 5 aa, unidentified
SSVHY

SEQ ID NO: 101 - skipped

SEQ ID NO: 102 - protein, 9 aa, unidentified
QQWTSNPPT

SEQ ID NO: 103 - protein, 123 aa, unidentified
ELILVESGGGLVKPGGSLKLSCAVSGFTFSSFAMSWVRQTPEKRLEWVATISSGGAYTFYKDSVKGRFTISRDNAKNTLYLQMSSLRSEDSAMYYCARHSGYDGYYLYAMDYWGQGTSVTVSS

SEQ ID NO: 104 - protein, 127 aa, unidentified
GVQCELILVESGGGLVKPGGSLKLSCAVSGFTFSSFAVSWVRQTPEKRLEWVATISSGGAYTFYKDSVKGRFTISRDNAKNTLYLQMSSLRSEDSAMYYCARHSGYDGYYLYAMDYWGQGTSVTVSS

SEQ ID NO: 105 - protein, 121 aa, unidentified
EVQLEESGGGLVQPGGSLKVSCAVSGLAFSSHDMSWVRQTPEKRLEWVAYISGGGTNIYYSDTVKGRFTISRDNAKNTLYLQMSSLKSEDTAIYYCARPNYNYGGSWFAYWGQGTLVTVSA

SEQ ID NO: 106 - protein, 106 aa, unidentified
QIVLTQSPEIMSASPGEKVTMTCSASSSVHYMHWYQQKSGTSPKRWIYDTSKLASGVPGRFSGSGSGTSYSLTISSMESEDAATYYCQQWTSNPPTFGGGTKLEIK

SEQ ID NO: 107 - protein, 244 aa, unidentified
QIVLTQSPEIMSASPGEKVTMTCSASSSVHYMHWYQQKSGTSPKRWIYDTSKLASGVPGRFSGSGSGTSYSLTISSMESEDAATYYCQQWTSNPPTFGGGTKLEIKGGGGSGGGGSGGGGSELILVESGGGLVKPGGSLKLSCAVSGFTFSSFAMSWVRQTPEKRLEWVATISSGGAYTFYKDSVKGRFTISRDNAKNTLYLQMSSLRSEDSAMYYCARHSGYDGYYLYAMDYWGQGTSVTVSS

SEQ ID NO: 108 - protein, 248 aa, unidentified
QIVLTQSPEIMSASPGEKVTMTCSASSSVHYMHWYQQKSGTSPKRWIYDTSKLASGVPGRFSGSGSGTSYSLTISSMESEDAATYYCQQWTSNPPTFGGGTKLEIKGGGGSGGGGSGGGGSGVQCELILVESGGGLVKPGGSLKLSCAVSGFTFSSFAVSWVRQTPEKRLEWVATISSGGAYTFYKDSVKGRFTISRDNAKNTLYLQMSSLRSEDSAMYYCARHSGYDGYYLYAMDYWGQGTSVTVSS

SEQ ID NO: 109 - protein, 242 aa, unidentified
QIVLTQSPEIMSASPGEKVTMTCSASSSVHYMHWYQQKSGTSPKRWIYDTSKLASGVPGRFSGSGSGTSYSLTISSMESEDAATYYCQQWTSNPPTFGGGTKLEIKGGGGSGGGGSGGGGSEVQLEESGGGLVQPGGSLKVSCAVSGLAFSSHDMSWVRQTPEKRLEWVAYISGGGTNIYYSDTVKGRFTISRDNAKNTLYLQMSSLKSEDTAIYYCARPNYNYGGSWFAYWGQGTLVTVSA

SEQ ID NO: 110 - protein, 8 aa, unidentified
GYTFTDYN

SEQ ID NO: 111 - protein, 8 aa, unidentified
INPNNGGT

SEQ ID NO: 112 - protein, 14 aa, unidentified
ARKGYGGNYDYFDY

SEQ ID NO: 113 - protein, 6 aa, unidentified
QSIGTS

SEQ ID NO: 114 - skipped

SEQ ID NO: 115 - protein, 9 aa, unidentified
QQSNSWPYT

SEQ ID NO: 116 - protein, 8 aa, unidentified
GFNIKDTY

SEQ ID NO: 117 - protein, 10 aa, unidentified
GFSLSTYGMG

SEQ ID NO: 118 - protein, 8 aa, unidentified
IDPANGNT

SEQ ID NO: 119 - protein, 7 aa, unidentified
IYWDDDK

SEQ ID NO: 120 - protein, 11 aa, unidentified
ALYYYGGSLDY

SEQ ID NO: 121 - protein, 14 aa, unidentified
AQSLIYDGYYGFAY

SEQ ID NO: 122 - protein, 12 aa, unidentified
QSLLYSGNQKNY

SEQ ID NO: 123 - skipped

SEQ ID NO: 124 - protein, 9 aa, unidentified
QQYYSYPRT

SEQ ID NO: 125 - protein, 8 aa, unidentified
GYTFTYYG

SEQ ID NO: 126 - protein, 8 aa, unidentified
INTYSGVP

SEQ ID NO: 127 - protein, 14 aa, unidentified
ARWIYYSDLYGMDY

SEQ ID NO: 128 - protein, 11 aa, unidentified
QSIVHSNGDTY

SEQ ID NO: 129 - skipped

SEQ ID NO: 130 - protein, 9 aa, unidentified
FQGSHVPWT

SEQ ID NO: 131 - protein, 8 aa, unidentified
GYTFSSYW

SEQ ID NO: 132 - protein, 8 aa, unidentified
GYTLTTYL

SEQ ID NO: 133 - protein, 8 aa, unidentified
INPSSGYT

SEQ ID NO: 134 - protein, 8 aa, unidentified
INPNSGSS

SEQ ID NO: 135 - protein, 13 aa, unidentified
ARDGNYDHWYFDV

SEQ ID NO: 136 - protein, 12 aa, unidentified
AIRHYGGSLFDY

SEQ ID NO: 137 - protein, 6 aa, unidentified
QDINSY

SEQ ID NO: 138 - protein, 12 aa, unidentified
QSLLNSRTRKNY

SEQ ID NO: 139-140 - skipped (2 entries)

SEQ ID NO: 141 - protein, 8 aa, unidentified
LQYDELLT

SEQ ID NO: 142 - protein, 8 aa, unidentified
EQSYNLFT

SEQ ID NO: 143 - protein, 121 aa, unidentified
EVQLQQSGPELVKPGSSVKISCKASGYTFTDYNMDWVKQSHGKSLEWIGTINPNNGGTSYNQKFKGKATLTVDKSSSTAYMELRSLTSEDSAVYYCARKGYGGNYDYFDYWGQGTTLTVSS

SEQ ID NO: 144 - protein, 107 aa, unidentified
DILLTQSPAILSVSPGERVSFSCRASQSIGTSIHWYQQRTNGSPRLLIKYASESISGIPSRFSGSGSGTDFTLSINSVESEDIADYYCQQSNSWPYTFGGGTKLEIK

SEQ ID NO: 145 - protein, 118 aa, unidentified
EVQLQQSGAELVKPGASVKLSCTASGFNIKDTYMHWVKQRPEQGLEWIGRIDPANGNTIYASKFQGKATITADTSSNTAYMQLSSLTSGDTAVYYCALYYYGGSLDYWGQGTTLTVSS

SEQ ID NO: 146 - protein, 122 aa, unidentified
QVTLKESGPGILQPSQTLSLTCSFSGFSLSTYGMGVSWIRQPSGKGLEWLAHIYWDDDKRYNPSLKSRLTISKDTSNNQVFLKITSVDTADTATYYCAQSLIYDGYYGFAYWGQGTLVTVSA

SEQ ID NO: 147 - protein, 121 aa, unidentified
QIQLVQSGPELKKPGETVKISCKASGYTFTYYGMNWVKQAPGKGLEWMGWINTYSGVPTYADDFKGRFAFSLETSVSTAYLQINNLKNEDTATYFCARWIYYSDLYGMDYWGQGTSVTVSS

SEQ ID NO: 148 - protein, 113 aa, unidentified
DIVMSQSPSSLAVSVGERVTMSCKSSQSLLYSGNQKNYLAWYQQKPGQSPKLLIYWASTRESGVPDRFTGSGSGTDFTLTISSVKAEDLAVYYCQQYYSYPRTFGGGTKLEIK

SEQ ID NO: 149 - protein, 112 aa, unidentified
DVLMTQSPLSLPVSLGDQASISCRSSQSIVHSNGDTYLEWYLQKPGQSPKLLIYKVSNRFSGVPDRFSGSGSGTDFTLKISRVEAEDLGVYHCFQGSHVPWTFGGGTKLEIK

SEQ ID NO: 150 - protein, 120 aa, unidentified
QVQLQQSGAELAKPGASVKMSCKASGYTFSSYWMHWLKQRPGQGLEWIGYINPSSGYTNYNQKFKDKATLTADKSSSTAYMQLSSLTSEDSAVYYCARDGNYDHWYFDVWGTGTTVTVSS

SEQ ID NO: 151 - protein, 119 aa, unidentified
QVQLQQPGAELVRPGASVKMSCKASGYTLTTYLMDWVKQRLGQGFEWIGNINPNSGSSNYNEKFKGKAKLTVDKSSSTAYMQLSSLTSEDSAVYYCAIRHYGGSLFDYWGQGTTLTVSS

SEQ ID NO: 152 - protein, 106 aa, unidentified
DIKMTQSPSSMYASLGERVTITCKASQDINSYLSWFQQKPGKSPKTLIYRANRLVDGVPSRFSGSGSGQDYSLTISSLEYEDMGIYYCLQYDELLTFGAGTKLELK

SEQ ID NO: 153 - protein, 112 aa, unidentified
DIVMSQSPSSLAVSAGERVTMSCRSSQSLLNSRTRKNYLAWYQQKPGQSPKLLIYWASTRESGVPDRFSGSGSGTDFTLTISSVQAEDLAVYYCEQSYNLFTFGSGTKLEIK

SEQ ID NO: 154 - protein, 243 aa, unidentified
EVQLQQSGPELVKPGSSVKISCKASGYTFTDYNMDWVKQSHGKSLEWIGTINPNNGGTSYNQKFKGKATLTVDKSSSTAYMELRSLTSEDSAVYYCARKGYGGNYDYFDYWGQGTTLTVSSGGGGSGGGGSGGGGSDILLTQSPAILSVSPGERVSFSCRASQSIGTSIHWYQQRTNGSPRLLIKYASESISGIPSRFSGSGSGTDFTLSINSVESEDIADYYCQQSNSWPYTFGGGTKLEIK

SEQ ID NO: 155 - protein, 246 aa, unidentified
EVQLQQSGAELVKPGASVKLSCTASGFNIKDTYMHWVKQRPEQGLEWIGRIDPANGNTIYASKFQGKATITADTSSNTAYMQLSSLTSGDTAVYYCALYYYGGSLDYWGQGTTLTVSSGGGGSGGGGSGGGGSDIVMSQSPSSLAVSVGERVTMSCKSSQSLLYSGNQKNYLAWYQQKPGQSPKLLIYWASTRESGVPDRFTGSGSGTDFTLTISSVKAEDLAVYYCQQYYSYPRTFGGGTKLEIK

SEQ ID NO: 156 - protein, 250 aa, unidentified
QVTLKESGPGILQPSQTLSLTCSFSGFSLSTYGMGVSWIRQPSGKGLEWLAHIYWDDDKRYNPSLKSRLTISKDTSNNQVFLKITSVDTADTATYYCAQSLIYDGYYGFAYWGQGTLVTVSAGGGGSGGGGSGGGGSDIVMSQSPSSLAVSVGERVTMSCKSSQSLLYSGNQKNYLAWYQQKPGQSPKLLIYWASTRESGVPDRFTGSGSGTDFTLTISSVKAEDLAVYYCQQYYSYPRTFGGGTKLEIK

SEQ ID NO: 157 - protein, 248 aa, unidentified
QIQLVQSGPELKKPGETVKISCKASGYTFTYYGMNWVKQAPGKGLEWMGWINTYSGVPTYADDFKGRFAFSLETSVSTAYLQINNLKNEDTATYFCARWIYYSDLYGMDYWGQGTSVTVSSGGGGSGGGGSGGGGSDVLMTQSPLSLPVSLGDQASISCRSSQSIVHSNGDTYLEWYLQKPGQSPKLLIYKVSNRFSGVPDRFSGSGSGTDFTLKISRVEAEDLGVYHCFQGSHVPWTFGGGTKLEIK

SEQ ID NO: 158 - protein, 241 aa, unidentified
QVQLQQSGAELAKPGASVKMSCKASGYTFSSYWMHWLKQRPGQGLEWIGYINPSSGYTNYNQKFKDKATLTADKSSSTAYMQLSSLTSEDSAVYYCARDGNYDHWYFDVWGTGTTVTVSSGGGGSGGGGSGGGGSDIKMTQSPSSMYASLGERVTITCKASQDINSYLSWFQQKPGKSPKTLIYRANRLVDGVPSRFSGSGSGQDYSLTISSLEYEDMGIYYCLQYDELLTFGAGTKLELK

SEQ ID NO: 159 - protein, 247 aa, unidentified
QVQLQQSGAELAKPGASVKMSCKASGYTFSSYWMHWLKQRPGQGLEWIGYINPSSGYTNYNQKFKDKATLTADKSSSTAYMQLSSLTSEDSAVYYCARDGNYDHWYFDVWGTGTTVTVSSGGGGSGGGGSGGGGSDIVMSQSPSSLAVSAGERVTMSCRSSQSLLNSRTRKNYLAWYQQKPGQSPKLLIYWASTRESGVPDRFSGSGSGTDFTLTISSVQAEDLAVYYCEQSYNLFTFGSGTKLEIK

SEQ ID NO: 160 - protein, 240 aa, unidentified
QVQLQQPGAELVRPGASVKMSCKASGYTLTTYLMDWVKQRLGQGFEWIGNINPNSGSSNYNEKFKGKAKLTVDKSSSTAYMQLSSLTSEDSAVYYCAIRHYGGSLFDYWGQGTTLTVSSGGGGSGGGGSGGGGSDIKMTQSPSSMYASLGERVTITCKASQDINSYLSWFQQKPGKSPKTLIYRANRLVDGVPSRFSGSGSGQDYSLTISSLEYEDMGIYYCLQYDELLTFGAGTKLELK

SEQ ID NO: 161 - protein, 246 aa, unidentified
QVQLQQPGAELVRPGASVKMSCKASGYTLTTYLMDWVKQRLGQGFEWIGNINPNSGSSNYNEKFKGKAKLTVDKSSSTAYMQLSSLTSEDSAVYYCAIRHYGGSLFDYWGQGTTLTVSSGGGGSGGGGSGGGGSDIVMSQSPSSLAVSAGERVTMSCRSSQSLLNSRTRKNYLAWYQQKPGQSPKLLIYWASTRESGVPDRFSGSGSGTDFTLTISSVQAEDLAVYYCEQSYNLFTFGSGTKLEIK

SEQ ID NO: 162 - protein, 5 aa, unidentified
SFAMS

SEQ ID NO: 163 - protein, 17 aa, unidentified
AISGSGGGTYYADSVKG

SEQ ID NO: 164 - protein, 13 aa, unidentified
DKILWFGEPVFDY

SEQ ID NO: 165 - protein, 11 aa, unidentified
RASQSVSSYLA

SEQ ID NO: 166 - protein, 7 aa, unidentified
DASNRAT

SEQ ID NO: 167 - protein, 10 aa, unidentified
QQRSNWPPTF

SEQ ID NO: 168 - protein, 122 aa, unidentified [sequence omitted]

SEQ ID NO: 169 - protein, 107 aa, unidentified [sequence omitted]

SEQ ID NO: 170 - protein, 244 aa, unidentified
EVQLLESGGGLVQPGGSLRLSCAVSGFTFNSFAMSWVRQAPGKGLEWVSAISGSGGGTYYADSVKGRFTISRDNSKNTLYLQMNSLRAEDTAVYFCAKDKILWFGEPVFDYWGQGTLVTVSSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIK

SEQ ID NO: 171 - protein, 5 aa, unidentified
SYWMH

SEQ ID NO: 172 - protein, 17 aa, unidentified
EIDPSDSYKDYNQKFKD

SEQ ID NO: 173 - protein, 9 aa, unidentified
AITTTPFDF

SEQ ID NO: 174 - protein, 11 aa, unidentified
RASQSISNNLH

SEQ ID NO: 175 - protein, 7 aa, unidentified
YASQSIS

SEQ ID NO: 176 - protein, 9 aa, unidentified
QQSNTWPYT

SEQ ID NO: 177 - protein, 118 aa, unidentified
QVQLQQPGAELVKPGASLKLSCKSSGYTFTSYWMHWVRQRPGHGLEWIGEIDPSDSYKDYNQKFKDKATLTVDRSSNTAYMHLSSLTSDDSAVYYCARAITTTPFDFWGQGTTLTVSS

SEQ ID NO: 178 - protein, 108 aa, unidentified
DIVLTQSPATLSVTPGDSVSLSCRASQSISNNLHWYQQKSHESPRLLIKYASQSISGIPSRFSGSGSGTDFTLSINSVETEDFGVYFCQQSNTWPYTFGGGTKLEIKR

SEQ ID NO: 179 - protein, 241 aa, unidentified
QVQLQQPGAELVKPGASLKLSCKSSGYTFTSYWMHWVRQRPGHGLEWIGEIDPSDSYKDYNQKFKDKATLTVDRSSNTAYMHLSSLTSDDSAVYYCARAITTTPFDFWGQGTTLTVSSGGGGSGGGGSGGGGSDIVLTQSPATLSVTPGDSVSLSCRASQSISNNLHWYQQKSHESPRLLIKYASQSISGIPSRFSGSGSGTDFTLSINSVETEDFGVYFCQQSNTWPYTFGGGTKLEIKR

SEQ ID NO: 180 - protein, 114 aa, unidentified
DIVMTQSPSSLSVSAGEKVTMSCKSSQSLLNSGNQKNYMAWYQQKPGQPPKLLIYGASTRESGVPDRFTGSGSGTDFTLTISSVQAEDLAVYYCQNDHSYPLTFGAGTKLELKR

SEQ ID NO: 181 - protein, 252 aa, unidentified
QVQLKQSGPGLVQPSQSLSITCTVSGFSLTNYGLHWVRQSPGKGLEWLGVIWSGGSTDYNAAFISRLSISKDNSKSQVFFKMNSLQADDTAIYYCARKGGIYYANHYYAMDYWGQGTSVTVSSGGGGSGGGGSGGGGSDIVMTQSPSSLSVSAGEKVTMSCKSSQSLLNSGNQKNYMAWYQQKPGQPPKLLIYGASTRESGVPDRFTGSGSGTDFTLTISSVQAEDLAVYYCQNDHSYPLTFGAGTKLELKR